(19) United States
(12) Patent Application Publication
(10) Pub. No.: US 2009/0075926 A1
(43) Pub. Date: Mar. 19, 2009

(54) METHOD FOR IDENTIFYING AND MANIPULATING CELLS

(76) Inventor: Cynthia C. Bamdad, Boston, MA (US)

Correspondence Address: JHK LAW, P.O. BOX 1078, LA CANADA, CA, 91012-1078, US

(21) Appl. No.: 11/951,613

(22) Filed: Dec. 6, 2007

Related U.S. Application Data
(60) Provisional application No. 60/868,914, filed on Dec. 6, 2006.
(60) Provisional application No. 60/917,292, filed on May 10, 2007.
(60) Provisional application No. 60/947,392, filed on Jun. 29, 2007.

(51) Int. Cl. A61K 31/7088; C12N 5/02; C07K 14/00; G01N 33/574

(52) U.S. Cl. 514/44; 435/375; 530/350; 435/4; 435/377; 435/7.23

(57) ABSTRACT

The present application discloses a method of isolating or selecting stem cells from a mixed population containing stem cells, which includes the population of cells with a ligand specific for a truncated MUC1 receptor, wherein the presence of the truncated MUC1 receptor on the cells indicates that they are stem cells.

### BACKGROUND OF THE INVENTION

1. Field of the Invention

The invention relates to a method for expanding a population of MUC1 expressing cells. The present invention also relates to methods and reagents for the identification, isolation, expansion and manipulation of MUC1 expressing cells, in particular, stem cells and pre-cursor cells.

2. General Background and State of the Art

The following documents are incorporated herein by reference: PCT Application No. PCT/US2004/027954 (WO 2005/019269), filed Aug. 26, 2004; PCT Publication No. WO 02/056022, published Jul. 18, 2002; U.S. patent application Ser. No. 09/996,069, filed Nov. 27, 2001, published as Publication No. 2003/0036199 on Feb. 20, 2003, which describe the role of MUC1 receptor in tumorigenesis.

Stem cells are a class of cells that can give rise to many different cell types, which in turn generate many different tissue types. An important characteristic of stem cells is that they have the ability to self-renew indefinitely and to differentiate into different types of adult cells. Progenitor cells are a later type of cell that also have the ability to differentiate into different kinds of cells, however, progenitor cells have lost the ability to become any type of cell.

There are three major classes of stem cells. Totipotent cells, such as a fertilized egg, are able to give rise to a complete, fully functional organism. Pluripotent stem cells are more specialized than totipotent stem cells in that they cannot produce a complete organism or person but they can give rise to every type of cell in the body. Multipotent stem cells can give rise to the types of cells that are found in the type of tissue from which they were derived. For example, blood multipotent cells can only generate specialized blood cells, while skin multipotent cells can only generate the various types of skin cells.

Stem cells have great potential for being used therapeutically. Conditions such as Alzheimer's, Parkinson's, diabetes, spinal cord injury, stroke and some types of birth defects are caused by the destruction of cells and tissues that the adult human body cannot regenerate. Since stem cells have the potential to become any cell type, they could be used to generate new cells and tissues to cure these conditions.

In order to realize the potential of stem cell therapies, one must be able to identify and isolate stem cells and in addition one must understand and possess the capability to manipulate the mechanisms that mediate stem cell renewal and differentiation. At this time, the knowledge of these mechanisms is in its infancy. There are at least three factors, at this time, that appear to play a role in stem cell differentiation: 1) contamination of populations of undifferentiated cells with differentiated cells; 2) adhesion to a surface; and 3) the addition of growth factors, some of which support stem cell self-renewal and some of which signal the cell to commit to a differentiation pathway. It is currently extremely difficult to identify and isolate pure populations of undifferentiated cells. Current methods involve assaying the cells for reactivity to an antibody against a certain protein that has been determined to be a “marker” for a particular differentiation state. More typically, cells are tested for the presence of a collection of markers, which together are indicative of a specific differentiation state. For example, the current method for identifying human pluripotent stem cells involves testing the cells for the presence of OCT4, SSEA4, TRA 1-60 and TRA 1-81. However, this method may also identify cells that are not truly pluripotent and that may be at the beginning of some differentiation pathway. These cells most likely secrete factors that influence the surrounding cells to commit to the same differentiation pathway. Therefore, a mixed or contaminated cell population would differentiate more quickly than a pure population and would not be useful for therapeutic implantation because the undifferentiated cells would be influenced to differentiate down a pathway that may eventuate in an undesirable cell type. For example, contamination of a pluripotent population with cells that have begun to differentiate into blood cells would not be ideal for implantation into the brain of a Parkinson's patient. Therefore, methods for identifying and isolating cells at discrete states of differentiation would be a vast improvement over the state of the art and would enable using stem cells therapeutically.

There is evidence that supports the idea that the adherence of stem cells to a surface promotes the differentiation process. Experiments in which stem cells were grown in a matrigel medium produced a higher percentage of differentiated colonies than the feeder layer method, perhaps because of the vast complexity of unidentified growth factors that are in the matrigel medium or perhaps due to surface effects. Therefore, methods which promote anchorage-independent cell growth would be useful for propagating undifferentiated stem cells.

Since it is not known which growth factors support renewal of pluripotency and which initiate differentiation, embryonic stem cells are currently grown in minimal media supplemented with FGF, which is a known growth factor that appears to promote self-renewal. In addition, the stem cells are grown over a layer of “feeder” cells, typically fibroblasts, which secrete other necessary, but as yet unidentified, factors that support stem cell growth. These feeder cells undoubtedly produce a milieu of growth factors, many of which also initiate differentiation, as evidenced by the fact that this method of growth produces a mixture of both undifferentiated and differentiated colonies. Therefore, it would be highly advantageous to identify specific growth factors that promote the regeneration of undifferentiated cells and to identify those factors that commit the cell to a differentiation pathway.

Therefore, there is considerable interest in developing methods to identify and isolate cells of distinct differentiation states, especially the state of pluripotency, in developing methods for growing undifferentiated cells anchorage-independently, and in determining which factors support their self-renewal process and which initiate differentiation. It would also be beneficial to develop methods to identify new sources of stem cells, be they embryonic or adult, toti, pluri, or multi-potent stem cells.

Recent research supports the existence of cancer stem cells (CSCs) (Prospective identification of tumorigenic stem cells. Al-Hajj M, Wicha M S, Benito-Hernandez A, Morrison S J and Clarke M F. (2003). Proc. Natl. Acad. Sci. USA, 100, 3983-3988; Characterization of clonogenic multiple myeloma cells. Matsui W, Huff C A, Wang Q, Malehorn M T, Barber J, Tanhehco Y, Smith B D, Civin Cland Jones R J. (2004) Blood, 103, 2332-2336; Identification of a cancer stem cell in human brain tumor. Singh S K, Clarke I D, Terasaki M, Bonn V E, Hawkins C, Squire J and Dirks P B. (2003) Cancer Res., 63, 5281-5828). CSCs are those cells that are sufficient for initiating the growth of a tumor, seeding a cancer in an otherwise cancer-free host or seeding a new cancer site in a host previously burdened with cancer. Normal stem cells are characterized by their ability to self-renew indefinitely and to differentiate to become adult cells of distinct tissue types. Progenitor cells have the ability to further differentiate into distinct cell types but have lost the ability to differentiate into any type of cell. It has been shown that not all cancer cells have the ability to self-renew, to induce disease in a new host, or to form new tumors (A cell initiating human acute myeloid leukaemia after transplantation into SCID mice. Lapidot T, Srirad C, Vormoor J, Murdoch B, Hoang T, Caceres-Cortes J, Minden M, Paterson B, Caligiuri M and Dick J. (1994). Nature, 17, 645-648; Identification of a cancer stem cell in human brain tumor. Singh S K, Clarke I D, Terasaki M, Bonn V E, Hawkins C, Squire J and Dirks P B. (2003) Cancer Res., 63, 5281-5828; Human acute myeloid leukemia is organized as a hierarchy that originates from a primitive hematopoietic cell. Bonnet D., and Dick J E. (1997) Nat. Med. 3, 730-737). Rather, only a small fraction of cancer cells have this ability to self-renew and form new tumors, i.e. metastasize. A leading theory is that cancer stem cells may operate in a fashion similar to normal stem cells (Self-renewal and solid tumor stem cells Al-Hajj M and Clarke M F (2004) Oncogene, 23, 7274-7282). Solid tumors occur in organs that have stem cell populations. Epithelial cancers, which include breast, prostate, colon, and lung cancers are the most common cancers in adults. Over 75% of these cancers are characterized by the aberrant expression of the MUC1 receptor (Development and characterization of breast cancer reactive monoclonal antibodies directed to the core protein of the human milk mucin Burchell J, Gendler S, Taylor-Papadimitriou J, Girling A, Lewis A, Millis R, and Lamport D. (1987) Cancer Res., 47, 5476-5482; Monoclonal antibodies to epithelial sialomucins recognize epitopes at different cellular sites in adenolymphomas of the parotid gland Zotter S, Hageman P C, Lossnitzer A, Mooi W and Hilgers J (1988) Cancer Rev. 11-12, 55-101; Mucins and mucin binding proteins in colorectal cancer Byrd J C and Bresalier R S (2004) Cancer Metastasis Review January-June; 23(1-2):77-99), wherein aberrant expression means that the receptor is no longer localized to the apical border of luminal cells but rather is uniformly distributed over the entire cell surface (Differential reactivity of a novel monoclonal antibody (DF3) with human malignant versus benign breast tumors (1984) Kufe D, Inghirami G, Abe, M, Hayes D, Justi-wheeler H and Schlom J Hybridoma, 3, 223-232). The greatest percentage of MUC1-positive cancers is in breast cancers where greater than 96% show aberrant MUC1 expression. Interestingly, in the adult female, breast tissue must undergo cyclic bursts of growth and apoptosis with each menstrual period and pregnancy. Thus it follows that breast tissue must maintain functional stem cell or at least progenitor cell populations throughout adult female life.

The identification of the growth factors and their receptors that drive the growth of cancer cells could provide the key to understanding how to grow and manipulate stem cells and progenitor cells for research, therapeutic and other uses.

MUC1 (mucin 1) is a transmembrane mucin glycoprotein that is expressed on a number of epithelial cell types (Molecular cloning and expression of the human tumor associated polymorphic epithelial mucin, PEM. Gendler S j, Lancaster C A, Taylor-Papadimitriou J, Dhuig, T, Peat, N, Burchell, J, Pemberton, L, Lalani, E-N and Wilson D. (1990) J. Biol. Chem. 265, 15286-15293; Episialin, a carcinoma associated mucin, is generated by a polymorphic gene encoding splice variants with alternative amino termini. Ligtenberg M J L, Vos H L, Genissen, A M C and Hilkens J. (1990) J. Biol. Chem. 265, 15573-15578), on haematopoietic cells (Evaluation of MUC1 and EGP40 in Bone marrow and Peripheral Blood as a Marker for Occult breast cancer (2001 Zhong X Y, Kaul S, Bastert G, Arch Gynecol Obstet 264:177-181), and on progenitor cells as well (Epithelial Progenitors in the Normal Human mammary Gland. Stingl J, Raouf A, Emerman J, and Eaves C. (2005). Journal of Mamary Gland Biology and Neoplasia, Vol. 10, No. 1, 49-59). The cell surface receptor MUC1 is present at the apical border of healthy epithelium, but is aberrantly expressed (spread over the entire cell surface) in a wide range of human solid tumors. It has been known for some time that the MUC1 receptor can be “shed” from the cell surface, as a portion of the exyracellular domain can be detected in the blood of breast cancer patients. The inventors previously disclosed that the portion of the MUC1 receptor that remains attached to the cell surface after cleavage, consisting primarily of PSMGFR, is the major growth factor receptor that mediates the growth of MUC1-positive cancer cells in vitro. Transfection of a variant MUC1 receptor comprised of the intact transmembrane and cytoplasmic domains, but having an ectodomain that terminates at the end of the PSMGFR sequence is sufficient to confer the ability of these cells to grow anchorage-independently.

In further detail, MUC1 comprises several regions termed herein as follows, recited in an order starting from the C-terminus and extending through the cell membrane and out into the extracellular domain. The basic structure of the MUC1 receptor is illustrated in FIG. 1. The receptor, as illustrated comprises: 1) cytoplasmic tail; 2) transmembrane section; 3) MGFR; 4) IBR, 5) Unique Region, 6) repeats, and N-terminus region comprising a signal peptide. For a detailed description of MUC1 and its function in normal and tumor cells, see PCT/US2005/032821, which is incorporated by reference herein, in its entirety for its description of the function and activity of cleaved MUC1 on the cell surface.

### SUMMARY OF THE INVENTION

In one aspect, the present invention is directed to methods and reagents for the identification, isolation and manipulation of stem cells. In another aspect, the present invention is directed to a method for stimulating or enhancing proliferation of a population of cells by activating MUC1 receptor on the cells. The activating may be carried out by contacting the cells with (i) an agent that multimerizes the MGFR portion of MUC1; (ii) an agent that increases the cleavage of MUC1 to the growth factor receptor form; or (iii) a ligand that activates the MGFR portion of the MUC1 receptor. The cells may be non-tumorous cells, preferably immature cells, such as stem cells, progenitor cells, endometrial cells, neutrophil pre-cursors or neutrophils. Further, in this method, the MUC1 receptor may be a cell surface attached cleavage product. The MUC1 cleavage product may be MGFR. Further, the MGFR may include PSMGFR. In this method, the MUC1 receptor may be activated by a multimerizing agent of the MUC1 receptor. Further, the multimerizing agent may be a bivalent agent. The bivalent agent may recognize a portion of the MGFR. Further, the bivalent agent may be a synthetic compound. The bivalent agent may be a dimeric ligand of MUC1. And still further, the bivalent agent may be an antibody. In the method described above, the agent that increases the cleavage may be an enzyme. In a preferred aspect, the enzyme may be TACE/ADAM17 or MMP14 also known as MT1-MMP.

In another aspect, the invention is directed to a method for treating a patient displaying symptoms of a low white blood count comprising administering to the patient an agent for activating MUC1 receptor in cells. The method may include administering to a subject who indicated need for such treatment, wherein activating is carried out by contacting the cells with (i) an agent that multimerizes the MGFR portion of MUC1; (ii) an agent that increases the cleavage of MUC1 to the growth factor receptor form; or (iii) a ligand that activates the MGFR portion of the MUC1 receptor.

In still another aspect, the invention is directed to a method for treating a patient, who displays symptoms indicating that a medicinal benefit would be achieved by causing immature cells to proliferate, with an agent that activates MUC1 receptor in cells. In this method, MUC1 may be activated by dimerizing the MGFR portion of the MUC1 receptor. MUC1 may also be activated by stimulating the cleavage of MUC1 such that the portion that remains attached to the cell surface consists essentially of the PSMGFR, preferably nat-PSMGFR. In this method also, MUC1 may be activated by stimulating the production of MUC1 or post-translationally modified MUC1. GCSF and GMCSF are known to stimulate the secretion of stem cells and progenitors cells from the bone marrow of a live subject. Therefore, GCSF and or GMCSF may be used with methods of the invention to render stem cells and progenitors more accessible to MUC1 modulating agents that affect the growth of stem cells, progenitor cells and some epithelial cells that rapidly divide such as those lining the luminal edge of ducts and those cells that line the respiratory and digestive tracts. Further in this method, the MUC1 may be activated by stimulating the production of MUC1 or post-translationally modified MUC1 by adding Granulocyte-Colony Stimulating Factor (G-CSF).

In yet another aspect, the invention is directed to a method for treating a patient, who displays symptoms that could be relieved by causing immature cells to proliferate by administering a DNA encoding (i) MUC1, (ii) a fragment of MUC1 that is displayed on the cell surface, or (iii) the MGFR portion of MUC1, to the patient at the site for which the cells are desired be proliferated.

In another aspect, the invention is directed to a method for stimulating proliferation of immature cells in vitro by introducing DNA encoding MUC1, a fragment of MUC1 that is displayed on the cell surface, or the MGFR portion of MUC1. In this method, the patient may be in treatment with chemotherapy agents for the treatment of MUC1-negative cancers.

The invention is also directed to a composition comprising: (i) an agent that multimerizes the MGFR portion of MUC1; (ii) an agent that increases the cleavage of MUC1 to the growth factor receptor form; or (iii) a ligand that activates the MGFR portion of the MUC1 receptor; and a pharmaceutically-acceptable carrier.

Applicant has discovered that MUC1* is expressed on the surface of human undifferentiated pluripotent embryonic stem cells. Since it is known that MUC1* functions as a growth factor receptor on cancer cells, it follows that MUC1* functions as a growth factor receptor on stem cells. Cellular adhesion may signal stem cells to differentiate.

In the inventive method, “normal” cells are grown anchorage-independently by transfecting in the MUC1* receptor. At certain receptor densities (it appears high densities self-signal), anchorage independent cell growth proceeds without stimulation; the addition of a bivalent anti-MUC1* antibody or other multimerizing agent, preferably a dimerizing agent, enhances this ability to grow anchorage-independently.

The applicant has demonstrated that the addition of the bivalent anti-MUC1* antibody (anti-PSMGFR and anti-PSMGFR-nat) to undifferentiated stem cells greatly enhances stem cell growth without inducing differentiation. The addition of bivalent anti-MGFR antibodies also enabled the anchorage independently growth of stem cells which it aids in staving off differentiation. The addition of bivalent anti-MGFR antibodies enhanced stem cell proliferation and did not require the addition of FGF or growth over fibroblast feeder cells. FGF, or +/−G-CSF or conditioned media may optionally be added with anti-MGFR antibodies to stimulate an even more enhanced stem cell growth.

Enhanced stem cell proliferation may also be accomplished by adding multimeric MUC1* ligand(s). NM23 has been shown by the inventor to stimulate the growth of MUC1*-positive cancer cells. It therefore follows then that the addition of NM23, particularly dimeric forms of NM23 may be used to activate the MUC1* receptor and stimulate stem cell growth.

The inventor has observed that undifferentiated embryonic stem cells exclusively express the cleaved form of MUC1, referred to herein as MUC1* or MGFR, and show no surface expression of full-length MUC1. However, it was discovered that the first step of stem cell differentiation involves a cessation of MUC1 cleavage. It was further discovered that cells go through a transition period when they express both full-length MUC1 (marker for differentiation) and OCT4 a previously identified marker of pluripotency. The applicants further observed that stem cells of high passage number often displayed all the known markers for pluripotency but also expressed full-length as well as cleaved MUC1. These results again support the conclusion that the known markers for pluripotency are insufficient and assessment of pluripotent state must include tests that detect the cleaved and full-length forms of MUC1. Therefore, antibodies against MUC1* and full-length MUC1 may be used together in a novel method for identifying and isolating stem cells of a distinct state of pluripotency or lack thereof.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 is a schematic of the full length MUC1 receptor and the growth factor receptor cleavage product, MGFR.

FIG. 2 is a graph of a cell proliferation assay in which three (3) different cells lines (A) breast cancer cell line 1504, (B) HeLa cells which are very slightly MUC1-positive and show a slight response in growth to MUC1 dimerization, and (C) HEK 293 cells which are MUC1-negative, were treated with anti-PSMGFR. Normalized cell growth is plotted as a function of antibody concentration. The growth curve of the MUC1-positive breast cancer cell line 1504 shows the typical biphasic response that is characteristic of a Class I growth factor receptor; cell growth is enhanced as antibody concentration is increased as each antibody dimerizes every two receptors. Cell growth begins to decline as antibody concentration becomes too high and each single antibody binds to a single receptor rather than dimerizing two receptors. Absent dimerization, the growth signal is lost. HEK 293 cells show no response to MUC1 stimulation by anti-PSMGFR since they are devoid of MUC1 receptors. These results indicate that the portion of the MUC1 receptor that contains the PSMGFR sequence functions as a growth factor receptor and stimulates the cell to divide when dimerized. Anti-Muc1* refers to anti-MGFR antibody.

FIG. 3 is a graph of a cell proliferation assay in which human embryonic kidney (HEK) 293 cells (MUC1-negative) that had been stably transfected with a MUC1 receptor that had a truncated ectodomain, terminated at the end of the PSMGFR sequence, were treated with anti-PSMGFR. Normalized cell growth is plotted as a function of antibody concentration and shows that the PSMGFR portion of the MUC1 receptor mediates cell growth via dimerization of this portion of the receptor.

FIG. 4 is a graph of a cell proliferation assay in which three (3) cell lines were treated with the monovalent-anti-PSMGFR which is incapable of dimerizing the receptor. The graph shows that the control cell lines (A) HeLa and (B) HEK 293s are unaffected by the addition of the antibody but in MUC1-positive cell line breast cancer cell line 1504 (C) and (D), cell growth is inhibited.

FIG. 5 is a western blot that shows that the ERK2 branch of the MAP kinase proliferation pathway is activated (ERK2 is phosphorylated) upon dimerization of the PSMGFR region of the MUC1 receptor.

FIG. 6 is a western blot of a competition experiment in which small molecules that bind to the PSMGFR region of MUC1 compete with anti-PSMGFR for binding to the site. In the presence of the competitor small molecule, the antibody does not bind and ERK2 phosphorylation is inhibited. These results indicate that the PSMGFR portion of the MUC1 receptor mediates cell growth and dimerization of the receptor can trigger this growth signal. The chemical formula for the competitor compound MN 9 referred to in this figure is

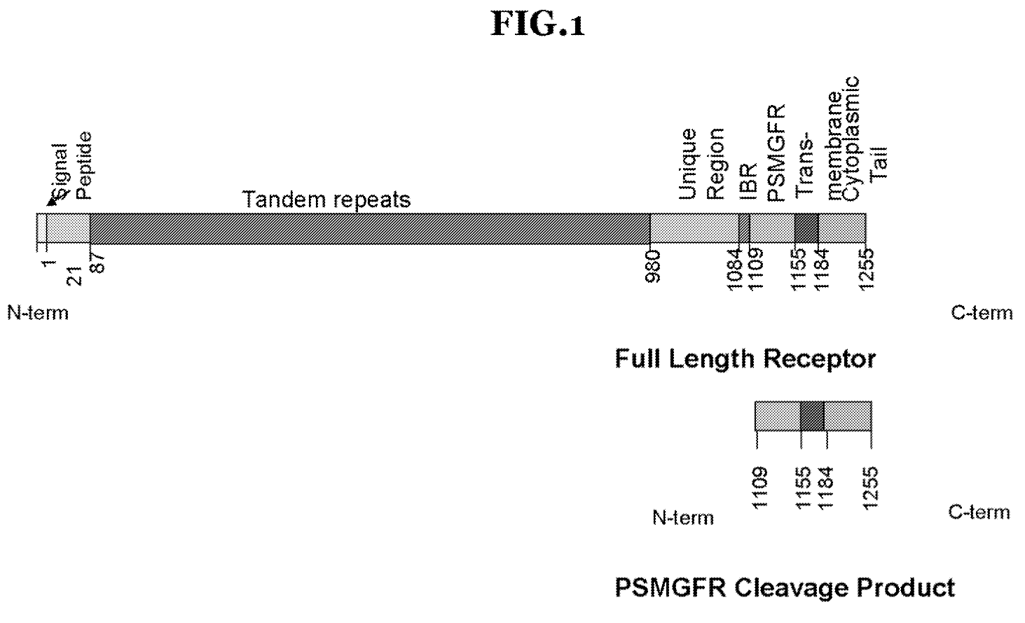

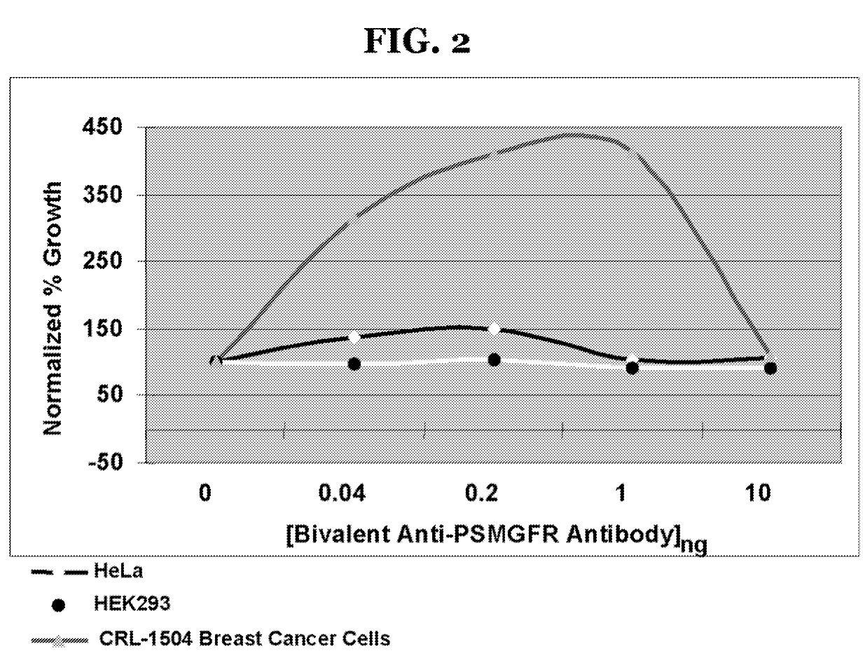

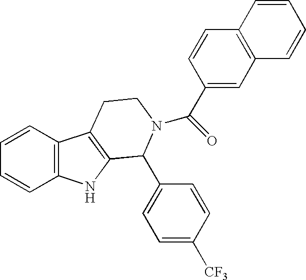

Compound MN 21 is

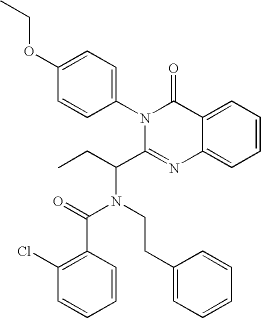

and Compound MN 13 is

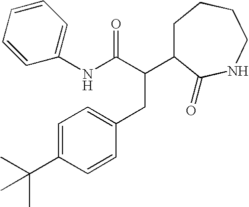

FIGS. 7A-7B show four (4) photographs of human breast cancer specimens under magnification. (A) and (C) are adjacent slices from the same section of a MUC1-positive cancer and (B) and (D) are adjacent slices from the same section of a MUC1-negative cancer. Sections (A) and (B) (top) have been treated with anti-PSMGFR that binds to the portion of the MUC1 receptor that remains attached to the cell surface after receptor cleavage. Sections (C) and (D) (bottom) have been treated with VU4H5 antibody that binds to the tandem repeat portion of the MUC1 receptor, which is frequently shed from the surface of cancer cells. Note the greater intensity of the anti-PSMGFR staining compared to VU4H5 staining. This result indicates that the predominant form of the MUC1 receptor on the surface of cancer cells is devoid of the tandem repeat portion and is comprised essentially of the PSMGFR sequence.

FIGS. 8A-8C show three (3) photographs of adjacent slices of a breast cancer biopsy specimen stained with either A) H&E; B) anti-PSMGFR, or C) VU4H5. Comparison of B) and C) show that VU4H5 stains the cytoplasm diffusely while anti-PSMGFR clearly stains the cell surface membrane. This indicates that, on cancer cells, the MUC1 receptor has been cleaved to release the tandem repeat portion but leaves the portion containing the PSMGFR sequence attached to the cell surface.

FIGS. 9A-9D show four (4) photographs of human lung cancer tissue specimens under magnification. (A) and (C) are adjacent slices from a first section of a MUC1-positive lung cancer and (B) and (D) are adjacent slices from a MUC1-negative cancer. Sections (A) and (B) (top) have been treated with anti-PSMGFR, which binds to the portion of the MUC1 receptor that remains attached to the cell surface after receptor cleavage. Sections (C) and (D) (bottom) have been treated with VU4H5 antibody that binds to the tandem repeat portion of the MUC1 receptor, which is frequently shed from the surface of cancer cells. Note the greater intensity of the anti-PSMGFR staining compared to VU4H5 staining and that anti-PSMGFR staining is restricted to the cell surface. These results again indicate that the predominant form of the MUC1 receptor on the surface of MUC1-positive lung cancer cells is mostly devoid of the tandem repeat portion and is comprised essentially of the PSMGFR sequence.

FIGS. 10A-10C show the same set of MUC1-positive lung cancer tissue specimens as in FIGS. 9A-9D at a greater magnification. At enhanced magnification, it is readily observed that the anti-PSMGFR staining is restricted to the cell surface whereas VU4H5 is diffuse and cytoplasmic, confirming that the MUC1 receptor on the surface of MUC1-positive lung cancer cells is cleaved to release the tandem repeat domain and leave the MGFR portion attached to the cell surface.

FIGS. 11A-11B show two (2) photographs of colon cancer tissue specimens that have been stained with either (A) anti-PSMGFR or (B) VU4H5. The arrows point to portions of the section that are very cancerous as indicated by the fact that they have lost all cellular architecture. Section (A), shows dark regions of staining with anti-PSMGFR but the same region of the adjacent section (B), which has been stained with VU4H5, which recognizes the tandem repeat portion of the MUC1 receptor, shows no staining at all. These results indicate that, in MUC1-positive colon cancer, the MUC1 receptor has been cleaved to release the tandem repeat portion but leaves the portion of the receptor that contains the PSMGFR sequence intact and attached to the cell surface.

FIGS. 12A-12B show two (2) photographs of MUC1-negative tissue specimens stained with either anti-PSMGFR (A) or VU4H5 (B). Note that in (A) arrows point to several mast progenitor cells, the surface of which have been thoroughly stained with anti-PSMGFR but not with VU4H5. These results indicate that a cleaved form of the MUC1 receptor that contains the PSMGFR sequence, but not the tandem repeat domain, is present on the surface of mast progenitor cells.

FIG. 13 is a greater magnification of FIG. 12 (A) and shows mast progenitor cells coated with anti-PSMGFR. Arrows point to mast progenitor cells coated with MUC1 cleavage product, PSMGFR.

FIGS. 14A-14B show photographs of adjacent slices of healthy fallopian tube tissue specimens stained with either anti-PSMGFR (A) that binds to cleaved MUC1 or VU4H5 (B) that binds to full-length MUC1.

FIGS. 15A-15F show human embryonic H9 stem cells (passage 40) immunolabeled with MUC1* and OCT4 antibodies. A) MUC1*. B) OCT4—area to the right of dotted line indicates an undifferentiated colony. C) Merged image of MUC1* and OCT4. D and E) are controls with primary antibodies omitted. F) DAPI stain of same cells shown in A-C. All images were photographed at 40× magnification.

FIGS. 16A-16F show human embryonic H9 stem cells (passage 40) immunolabeled with full length MUC1 and OCT4 antibodies. A) MUC1 full length. B) OCT4—area to the right of dotted line indicates an undifferentiated colony. C) Merged image of MUC1 full length and OCT4. D and E) are controls with primary antibodies omitted. F) DAPI stain of same cells shown in A-C. All images were photographed at 20× magnification.

FIGS. 17A-17E show undifferentiated H9 cells (passage 40) immunolabeled with MUC1* and SSEA4 antibodies. A MUC1*. B SSEA4. C Merged images of MUC1* and SSEA4. D and E are controls with primary antibodies omitted. All images were photographed at 40× magnification.

FIGS. 18A-18E show CRL-1500 breast cancer cell line immunolabeled with antibodies that bind to MUC1 full length and antibodies that bind to the cleaved form, MUC1*. A) MUC1. B) SRY. C) Merged images of MUC1 full length and MUC1*. D and E) are controls with primary antibodies omitted. All images were photographed at 40× magnification.

FIGS. 19A-F show double staining experiments to indicate that the MUC1* receptor co-localizes with the pluripotent markers SSEA4, TRA 1-60 and TRA 1-81.

FIGS. 20a (A-I) and 20b (A-G) show transition areas of H9 cells that were grown for 21 days without the addition of bFGF to induce the onset of differentiation; cells in transition areas expressed OCT4 and also showed surface staining for both MUC1* and for full length MUC1.

FIG. 21a (A-F) and 21b (A-F) show undifferentiated human embryonic stem cells that have been grown over fibroblast feeder cells (a) or over matrigel (b); the image shows that under both growth conditions, the cleavage enzymes TACE/ADAM17 and MT1-MMP/MMP14 are present on undifferentiated stem cells, which also present the cleaved form of the MUC1 receptor which is the growth factor receptor form.

FIG. 22 shows results of a binding reaction between breast cancer cell lysates and MUC1* attached to gold nanoparticles.

FIG. 23 is a photo of an immunoblot that shows that a species immunoprecipitated from breast cancer cells with a MUC1* peptide, reacts with an antibody against NM23.

FIG. 24 is a photo of an immunoblot that shows feedback expression of NM23 in response to MUC1* expression.

FIG. 25 shows that stem cells treated with monovalent antibody against MUC1*, which binds to a single MUC1* receptor and prevents dimerization, died as visualized by staining with Calcein which stains live cells and Ethidium which stains dead cells.

FIG. 26 is a photo of a live/dead cell assay that shows that stem cells treated with a monovalent anti-MUC1* resulted in total stem cell death.

FIG. 27 is a photo of a live/dead assay in which stem cells were treated with monovalent anti-MUC1* plus a MUC1* free peptide which resulted in an increase in the number of live cells compared to when the free peptide was not added.

FIG. 28 shows that stem cells treated with the bivalent anti-MUC1* antibody did not have a harmful effect on cell growth and may have stimulated cell growth.

FIG. 29 shows stem cell growth/death in the absence of the bivalent anti-MUC1* antibody.

FIG. 30 is a photo of a live/dead cell assay after stem cells were treated with bivalent anti-MUC1* antibody in the absence of added bFGF.

FIG. 31 is a photo of a live/dead cell assay of stem cells to which neither bivalent anti-MUC1* nor bFGF were added.

FIG. 32 is a bar graph that summarizes the results of a quantitative stem cell growth assay after treatment with bivalent anti-MUC1*, monovalent anti-MUC1* or no antibody added.

FIG. 33 is a bar graph that shows the results of stem cell growth for a variety of antibody conditions.

### DETAILED DESCRIPTION OF THE PREFERRED EMBODIMENTS

In the present application, “a” and “an” are used to refer to both single and a plurality of objects.

The term “MUC1 Growth Factor Receptor” (MGFR) is a functional definition meaning that portion of the MUC1 receptor that interacts with an activating ligand, such as a growth factor or a modifying enzyme such as a cleavage enzyme, to promote cell proliferation. The MGFR region of MUC1 is that extracellular portion that is closest to the cell surface and is defined by most or all of the PSMGFR, as defined below. The MGFR is inclusive of both unmodified peptides and peptides that have undergone enzyme modifications, such as, for example, phosphorylation, glycosylation, etc. Results of the invention are consistent with a mechanism in which this portion is made accessible to the ligand upon MUC1 cleavage at a site associated with tumorigenesis that causes release of the some or all of the IBR from the cell. MGFR is also known as MUC1*.

As used herein, “anti-PSMGFR” refers to any antibody that recognizes a region of the MGFR and optionally any portion of PSMGFR. Antibody to nat-PSMGFR is exemplified and preferred in the application, but is not meant to be limited to an antibody made against this specific sequence, as other fragments of MGFR and PSMGFR are also contemplated.

The term “Interchain Binding Region” (IBR) is a functional definition meaning that portion of the MUC1 receptor that binds strongly to identical regions of other MUC1 molecules giving MUC1 the ability to aggregate (i.e. self-aggregate) with other MUC1 receptors via the IBRs of the respective receptors. This self-aggregation may contribute to MUC1 receptor clustering, observed in healthy cells. In a preferred embodiment, the IBR may be approximately defined as a stretch of at least 12 to 18 amino acid sequence within the region of the full-length human MUC1 receptor defined as comprising amino acids 507 to 549 of the extracellular sequence of the MUC1 receptor (SEQ ID NO:1), with amino acids 525 through 540 and 525 through 549 especially preferred (numbers refer to Andrew Spicer et al., J. Biol. Chem. Vol 266 No. 23, 1991 pgs. 15099-15109; these amino acid numbers correspond to numbers 1067, 1109, 1085, 1100, 1085, 1109 of Genbank accession number P15941; PID G547937, SEQ ID NO:1) or fragments, functional variants or conservative substitutions thereof, as defined in more detail below.

The term “cleaved IBR” means the IBR (or a portion thereof) that has been released from the receptor molecule segment which remains attached to the cell surface. The release may be due to enzymatic or other cleavage of the IBR. As used herein, when the IBR is “at the surface of a cell”, it means the IBR is attached to the portion of the cell surface receptor that has not been shed, or cleaved. The cleaved IBR of interest is a “disease-associated cleavage”, i.e. that type of cleavage that can result in cancer.

The term “Constant Region” (CR) is any non-repeating sequence of MUC1 that exists in a 1:1 ratio with the IBR and forms part of the portion of MUC1 that is shed upon cleavage in healthy and tumorigenesic cells.

The term “Repeats” is given its normal meaning in the art.

The term “Primary Sequence of the MUC1 Growth Factor Receptor” (PSMGFR) is a peptide sequence that defines most or all of the MGFR in some cases, and functional variants and fragments of the peptide sequence, as defined below. The PSMGFR is defined as SEQ ID NO:10 listed below in Table 1, and all functional variants and fragments thereof having any integer value of amino acid substitutions up to 20 (i.e. 1, 2, 3, 4, 5, 6, 7, 8, 9, 10, 11, 12, 13, 14, 15, 16, 17, 18, 19, or 20) and/or any integer value of amino acid additions or deletions up to 20 at its N-terminus and/or C-terminus. A “functional variant or fragment” in the above context refers to such variant or fragment having the ability to specifically bind to, or otherwise specifically interact with, ligands that specifically bind to, or otherwise specifically interact with, the peptide of SEQ ID NO:10. One example of a PSMGFR that is a functional variant of the PSMGFR peptide of SEQ NO:10 (referred to as nat-PSMGFR—for “native”) is SEQ NO:12 (referred to as var-PSMGFR), which differs from nat-PSMGFR by including an -SPY- sequence instead of the native -SRY- (see bold text in sequence listings). Var-PSMGFR may have enhanced conformational stability, when compared to the native form, which may be important for certain applications such as for antibody production. The PSMGFR is inclusive of both unmodified peptides and peptides that have undergone enzyme modifications, such as, for example, phosphorylation, glycosylation, etc.

The term “Extended Sequence of the MUC1 Growth Factor Receptor” (ESMGFR) is a peptide sequence, defined below (See Table 1—SEQ ID NO:15), that defines all of His-var-PSMGFR plus 9 amino acids of the proximal end of PSIBR.

The term “Tumor-Specific Extended Sequence of the MUC1 Growth Factor Receptor” (TSESMGFR) is a peptide sequence (See, as an example, Table 1—SEQ ID NO:16) that defines a MUC1 cleavage product found in tumor cells that remains attached to the cell surface and is able to interact with activating ligands in a manner similar to the PSMGFR.

PSIBR is a peptide sequence, defined below (See Table 1—SEQ ID NO:17), that defines most or all of the IBR.

“Truncated Interchain Binding Region” (TPSIBR) is a peptide sequence defined below (See Table 1—SEQ ID NO:18), that defines a smaller portion of the IBR that is released from the cell surface after receptor cleavage in some tumor cells.

PSMGFRTC is a truncated MUC1 receptor isoform comprising PSMGFR and truncated at or within about up to 30 (i.e. within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) amino acids of its N-terminus and comprising the transmembrane and cytoplasmic sequences of full-length MUC1 receptor. As used herein, the phrase “at its N-terminus” referring to the location of a recited sequence within a larger molecule, such as a polypeptide or receptor, refers to such a sequence being no more than 30 amino acids from the N-terminal amino acid of the molecule. Optionally the PSMGFRTC, as well as the other truncated MUC1 receptor isoforms discussed below, can include a MUC1 N-terminal signaling sequence (Table 1—SEQ ID NOS: 2, 3, or 4), typically between 20 and 30 amino acids in length, or a functional fragment or variant thereof. Such a sequence is typically encoded by the nucleic acid constructs encoding a truncated MUC1 receptor. Such a PSMGFRTC, i.e. including the optional signal sequence, would still be a peptide or protein “having a PSMGFR” sequence “at its N-terminus” by the above definition. An example is nat-PSMGFRTC (SEQ ID NO:5, with or without the signal peptide of SEQ ID NOS: 2, 3, or 4 at the extreme N-terminus) having nat-PSMGFR (SEQ NO: 10) at its N-terminus (i.e. at the extreme N-terminal end or within 30 amino acids thereof).

As used herein, “multimerization” of the receptors includes without limitation dimerization of the receptors. Further, multimerization includes binding of co-receptor or co-receptors with MUC1, or binding of multiple MUC1 receptors with each other, which may be gathered together by a ligand or ligands possessing multiple valences.

A “ligand” to a cell surface receptor, refers to any substance that can interact with the receptor to temporarily or permanently alter its structure and/or function. Examples include, but are not limited to binding partners of the receptor, (e.g. antibodies or antigen-binding fragments thereof), and agents able to alter the chemical structure of the receptor (e.g. modifying enzymes).

An “activating ligand” refers to a ligand able interact with a receptor to transduce a signal to the cell. Activating ligands can include, but are not limited to, species that effect inductive multimerization of cell surface receptors such as a single molecular species with greater than one active site able to bind to a receptor; a dimer, a tetramer, a higher multimer, a bivalent antibody or bivalent antigen-binding fragment thereof, or a complex comprising a plurality of molecular species. Activating ligands can also include species that modify the receptor such that the receptor then transmits a signal. Enzymes can also be activating ligands when they modify a receptor to make it a new recognition site for other activating ligands, e.g. glycosylases are activating ligands when the addition of carbohydrates enhances the affinity of a ligand for the receptor. Cleavage enzymes are activating ligands when the cleavage product is the more active form of the receptor, e.g. by making a recognition site for a ligand more accessible. In the context of MUC1 stem cells or progenitor cells, an activating ligand can be a species that cleaves MUC1, chemically modifies the receptor, or species that interact with the MGFRs on the surface of the MUC1 cells to transduce a signal to the cell that stimulates proliferation, e.g. a species that effects inductive multimerization.

A “growth factor” refers to a species that may or may not fall into a class of previously-identified growth factors, but which acts as a growth factor in that it acts as an activating ligand.

A “MUC1 presenting cell” refers to cells expressing MUC1 and/or MGFRs on the surface.

The term “immature cell” is used herein to refer to cells that are in various stages of differentiation from undifferentiated stem cells to progenitor cells and other cells such as various pre-cursor cells and neutrophils, which are partially differentiated, and excludes cells that are fully differentiated.

The term, “stem cell” refers to a cell with capability of multi-lineage differentiation and self-renewal, as well as the capability to regenerate tissue. Stem cells may originate from but not limited to umbilical cord blood, liver stem cells, pancreatic stem cells, neuronal stem cells, bone marrow stem cells, peripheral blood stem cells, or a mixture thereof. Further, the invention is not limited to transplantation of any particular stem cell obtained from any particular source, but may include stem cells from “multiple stem cell sources” in mixture with one another. Thus, expanded mesenchymal stromal cells may be used in cotransplantation of the stem cells obtained from single or multiple stem cell sources to increase the amount of engraftment.

The term “cancer”, as used herein, may include but is not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Preferred cancers are; breast, prostate, lung, ovarian, colorectal, and brain cancer.

The term “cancer treatment” as described herein, may include but is not limited to: chemotherapy, radiotherapy, adjuvant therapy, or any combination of the aforementioned methods. Aspects of treatment that may vary include, but are not limited to: dosages, timing of administration, or duration or therapy; and may or may not be combined with other treatments, which may also vary in dosage, timing, or duration. Another treatment for cancer is surgery, which can be utilized either alone or in combination with any of the aforementioned treatment methods. One of ordinary skill in the medical arts may determine an appropriate treatment.

An “agent for prevention of cancer or tumorigenesis” means any agent that counteracts any process associated with cancer or tumorigenesis described herein.

An “agent that enhances cleavage of a cell surface receptor interchain binding region” as used herein is any composition that promotes cleavage at a particular location by modifying MUC1 with sugar groups or phosphates that create a recognition motif for cleavage at that location. Other enzymes can promote cleavage of receptors by activating other cleavage enzymes. One way to select agents that enhance cleavage of a cell surface receptor IBR is to first identify enzymes that affect cleavage as described above, and screen agents, and their analogs, for their ability to alter the activity of those enzymes. Another way is to test agents that are known to affect the activity of similar enzymes (e.g. from the same family) for their ability to alter the site of cleavage of MUC1, and to similarly test analogs of these agents. Alternatively, agents are screened in a cell-free assay containing the enzyme and MUC1 receptors, and the rate or position of cleavage measured by antibody probing, Polymerase Chain Reaction (PCR), or the like. Alternatively, without first identifying enzymes that affect MUC1, agents are screened against cells that present MUC1 for the agents' ability to alter cleavage site or the rate of cleavage of MUC1. For example, agents can be screened in an assay containing whole cells that present MUC1 and aggregation potential of the cell supernatant can be measured, an indication of the amount of IBR that remains attached to the cleaved portion of MUC1, i.e. the degree of cleavage between MGFR and IBR. In another technique, agents can be screened in an assay containing whole cells that present MUC1, the supernatant removed, and the cell remain tested for accessibility of the MGFR portion, e.g. using a labeled antibody to the MGFR. Agents can be identified from commercially available sources such as molecular libraries, or rationally designed based on known agents having the same functional capacity and tested for activity using the screening assays.

An “agent that enhances cleavage of the MUC1 receptor” is any composition that promotes or enhances cleavage of the MUC1 receptor at any location. Such an agent can be used to increase the population of stem cell or progenitor cells, which if cleavage is effected, then the accessibility of the MGFR, a functional receptor associated with cell proliferation, is enhanced or promoted. Such agents can be selected by exposing cells to a candidate agent and determine, in the supernatant, the amount of cleaved MUC1 receptor, relative to a control.

As used herein, for cells to “proliferate”, it is meant that the cells survive and their growth is stimulated.

A subject, as used herein, refers to any mammal (preferably, a human), and preferably a mammal that has a disease that may be treated by administering stem cells or progenitor cells to a site within the subject. Examples include a human, non-human primate, cow, horse, pig, sheep, goat, dog, or cat. Generally, the invention is directed toward use with humans.

The samples used herein are any body tissue or body fluid sample obtained from a subject. Preferred are body fluids, for example lymph, saliva, blood, urine, milk and breast secretions, and the like. Blood is most preferred. Samples of tissue and/or cells for use in the various methods described herein can be obtained through standard methods including, but not limited to: tissue biopsy, including punch biopsy and cell scraping, needle biopsy, and collection of blood or other bodily fluids by aspiration or other methods.

MUC1 Receptor

The inventors previously disclosed that a form of the MUC1 receptor that has a shortened extracellular domain is highly expressed on cancer cells and that it is the growth factor receptor activity of this truncated MUC1 that drives the growth of cancer cells. The extracellular domain of the truncated MUC1 consists primarily of the PSMGFR sequence, as shown in Table 1, SEQ ID NO:11, but has the transmembrane and cytoplasmic domains of MUC1 and is referred to herein as MUC1* and also as MGFR. In previous applications, it has been referred to as the MGFR. The shortened form of MUC1 receptor is most often the result of a cleavage event. However, MUC1 variants with truncated extracellular domains, such as MUC1/Y, can also be produced by alternative splicing and the like.

The present application discloses that the shortened form of the MUC1 receptor, containing primarily the PSMGFR, exists on human embryonic stem cells, on pluripotent stem cells, is a marker for pluripotency, and more broadly exists on cells that may yet undergo another step of differentiation. A proteolyzed form of MUC1, consisting primarily of the PSMGFR is present on intestinal mucosa, pluri-potent bone marrow stem cells, neutrophil pre-cursors, neutrophils and progenitor cells. The present application further discloses that the abbreviated form of MUC1 (MGFR or MUC1*) is a primal growth factor receptor that drives the growth of stem cells and progenitor cells. Similar to its function on cancer cells, stimulation of the MUC1* portion of the receptor accelerates stem cell and progenitor cell growth and inhibits cellular differentiation. In contrast, inhibition of the MUC1* portion inhibits growth and leads to cell death. Further, the addition of a MUC1* dimerizing agent inhibits cellular differentiation, while the withholding of said dimerizing agent promotes differentiation.

MUC1 Expression in Tumor Cells

It has been estimated that MUC1 is aberrantly expressed on 75% of all human solid tumors and may exist in other types of cancer as well. In this context, aberrant expression has historically referred to the observation that on healthy epithelium the receptor is clustered at the apical border while on cancer cells, the receptor is uniformly distributed over the entire cell surface. It has also been known for some time that a portion of the receptor can be detected in the blood of late stage breast cancer patients. In addition, there were reports in the literature describing possible cleavage sites in the MUC1 extracellular domain.

To establish a possible link between cancer and MUC1 cleavage and to determine which MUC1 species was expressed on the surface of cancer cells, a panel of human cancerous tissue specimens were probed with two antibodies: one that recognized a portion of the receptor that should be released from the cell surface after receptor cleavage and another that recognized a portion that should remain attached to the cell surface. The first antibody is a rabbit polyclonal antibody raised against the PSMGFR, referred to herein as anti-PSMGFR and also as anti-MUC1*. The second antibody is a commercially available antibody (VU4H5) that binds to the tandem repeats of the MUC1 receptor that are at N-terminal end of the receptor and distal to the cell surface. It should be noted that the MUC1 receptor contains hundreds of the tandem repeat motifs, so that each full-length receptor will be bound by hundreds of VU4H5 antibodies. In sharp contrast, the sequence to which anti-MUC1* binds occurs only once per receptor.

FIGS. 7, 9, 10 and 11 are human cancerous tissue specimens. The dual antibody staining experiment shows that most of the MUC1 on cancerous tissue has been cleaved to release the tandem repeat portion and leaves the MGFR or MUC1* portion attached to the cell surface. The predominant MUC1 species expressed on cancer cells reacts with anti-PSMGFR but not with VU4H5. FIG. 7 shows four (4) photographs of human breast cancer specimens under magnification. (A) and (C) are adjacent slices from the same section of a MUC1-positive cancer and (B) and (D) are adjacent slices from the same section of a MUC1-negative cancer. Sections (A) and (B) (top) have been treated with anti-PSMGFR. Sections (C) and (D) (bottom) have been treated with VU4H5 antibody that binds to the tandem repeat portion of the MUC1 receptor, which is frequently shed from the surface of cancer cells. Note the greater intensity of the anti-PSMGFR staining compared to VU4H5 staining. This result indicates that the predominant form of the MUC1 receptor on the surface of cancer cells is devoid of the tandem repeat portion and is comprised essentially of the PSMGFR sequence. FIG. 9 shows four (4) photographs of human lung cancer tissue specimens under magnification. (A) and (C) are adjacent slices from a first section of a MUC1-positive lung cancer and (B) and (D) are adjacent slices from a MUC1-negative cancer. Sections (A) and (B) (top) have been treated with anti-PSMGFR, which binds to the portion of the MUC1 receptor that remains attached to the cell surface after receptor cleavage. Sections (C) and (D) (bottom) have been treated with VU4H5 antibody that binds to the tandem repeat portion of the MUC1 receptor, which is frequently shed from the surface of cancer cells.

Note the greater intensity of the anti-PSMGFR staining compared to VU4H5 staining and that anti-PSMGFR staining is restricted to the cell surface. Note also that there is heavy cytoplasmic staining for MUC1 and no surface staining, when probing with VU4H5 (FIGS. 7C, 8C, and 10C). However, probing of the adjacent tissue slice with anti-PSMGFR showed that the entire cell surface was uniformly coated with a cleaved MUC1 that did not contain the tandem repeat section but did contain the PSMGFR sequence (FIGS. 7A, 8B, and 10B). These results again indicate that the predominant form of the MUC1 receptor on the surface of MUC1-positive lung cancer cells is mostly devoid of the tandem repeat portion and is comprised essentially of the PSMGFR sequence. FIG. 11 shows two (2) photographs of colon cancer tissue specimens that have been stained with either (A) anti-PSMGFR or (B) VU4H5. The arrows point to portions of the section that are very cancerous as indicated by the fact that they have lost all cellular architecture. Section (A), shows dark regions of staining with anti-PSMGFR but the same region of the adjacent section (B), which has been stained with VU4H5, which recognizes the tandem repeat portion of the MUC1 receptor, shows no staining at all. These results indicate that, the fastest growing portions of the tumor present a form of MUC1 that is devoid of the tandem repeat portion but leaves the portion of the receptor that contains the nat-PSMGFR sequence intact and attached to the cell surface. The results of these staining experiments show that the major MUC1 species on the surface of human cancerous tissue is MUC1*, while there is a lesser amount of full-length MUC1 on the cell surface, implying a high degree of MUC1 receptor cleavage in cancer.

To determine whether cultured cancer cell lines displayed ratios of cleaved MUC1 to uncleaved MUC1 that were similar to the ratio observed on cancerous tissue specimens, we analyzed MUC1-positive tumor cell lysates by SDS-PAGE. As we had observed for the cancerous tissue specimens, the vast majority of the MUC1 receptors on cultured tumor cell lines have been cleaved to leave the MUC1* portion attached to the cell surface, while a smaller percentage of the receptors includes the tandem repeat portion. Human-derived cancer cell lines that are known to have high expression levels of MUC1 were subjected to western blot analysis A low percentage acrylamide gel (6%) showed a high molecular weight protein band at about 220 kDa that reacted with the VU4H5 antibody. A high percentage gel (12%) showed a low molecular weight species that ran with an apparent molecular weight between 20 and 30 kDa that reacted with the anti-PSMGFR antibody. These data taken together with data presented above indicate that both cultured cancer cell lines and human cancerous tissue display high expression of a low molecular weight species that reacts with an antibody raised against the PSMGFR sequence.

The inventor previously disclosed that the MUC1* portion of the MUC1 receptor functions as a growth factor receptor. Further supporting that disclosure, data is presented herein that shows that transfection of the MUC1* portion of the receptor into MUC1-negative host cells is sufficient to cause those cells to grow at a faster rate, renders the cells resistant to cell death induced by standard chemotherapy drugs, and causes more cells to be in the G2/M phase of the cell cycle. These data taken together argue strongly that the MUC1* portion of the receptor functions as a growth factor receptor and can do so in previously MUC1-negative cells and in cells that are not tumor cells.

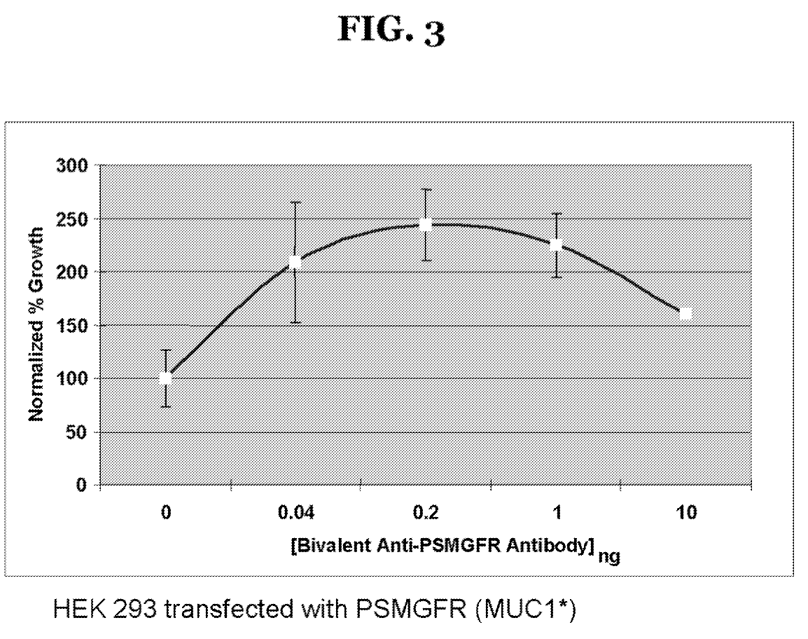

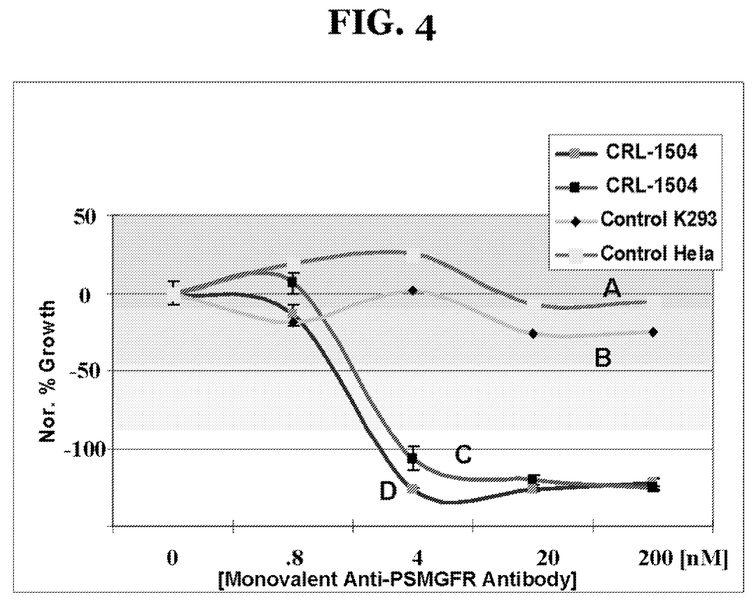

Many cell surface receptors are Class I growth factor receptors and are activated when the extracellular portions are brought close together or dimerized. To explore whether or not MUC1 functioned this way, bivalent anti-PSMGFR was used to dimerize the receptor. FIG. 2 is a graph of a cell proliferation assay in which three (3) different cells lines (A) breast cancer cell line 1504, (B) HeLa cells which are very slightly MUC1-positive and show a slight response in growth to MUC1 dimerization, and (C) HEK 293 cells which are MUC1-negative, were treated with anti-PSMGFR. Normalized cell growth is plotted as a function of antibody concentration. The growth curve of the MUC1-positive breast cancer cell line 1504 shows the typical biphasic response that is characteristic of a Class I growth factor receptor; cell growth is enhanced as antibody concentration is increased as each antibody dimerizes every two receptors. Cell growth begins to decline as antibody concentration becomes too high and each single antibody binds to a single receptor rather than dimerizing two receptors. Absent dimerization, the growth signal is lost. HEK 293 cells show no response to MUC1 stimulation by anti-PSMGFR since they are devoid of MUC1 receptors. Thus if a receptor functions as a Class I growth factor receptor, i.e. via receptor dimerization, then a plot of cell growth as a function of concentration of the added dimerizing agent will produce a bell-shaped curve. FIGS. 2 and 3 show this classic bell-shaped curve. These results indicate that the portion of the MUC1 receptor that contains the PSMGFR sequence functions as a growth factor receptor and stimulates the cell to divide when dimerized.

A key mechanism of cell growth in MUC1 positive cancers may depend more on the amount of MUC1 cleavage that occurs rather than the overall amount of MUC1 receptor that is expressed. Low molecular weight species that migrate on an acrylamide gel with an apparent molecular weight of around 20-30 kD (some glycosylated) exist in MUC1-positive tumor cells but do not exist in sufficient numbers to be detectable in non-tumor MUC1 cells. Two cleavage sites of the MUC1 receptor in tumor cells were previously identified. The first cleavage site occurs in the middle of the IBR and the second cleavage site, which our evidence indicates is the more tumorigenic form, occurs at the C-terminal end of the IBR: the first cleavage site being located at the N-terminus of TPSIBR (SEQ ID NO:17) and the second cleavage site being located at the N-terminus of the nat-PSMGFR having SEQ ID NO:13. When cleavage occurs at the first site, the portion of the receptor that remains attached to the cell surface is similar to TSESMGFR (See Table 1, SEQ ID NO:16, but with the native SRY sequence). When cleaved at the second site, the remaining portion is a PSMGFR as shown in Table 1, SEQ ID NO:11. This low molecular weight species that is tumor specific consists essentially of the native PSMGFR sequence and in some cases the TSESMGFR sequence and is available to cognate ligands, i.e. not self-aggregated, than on the overall amount of MUC1 receptor expressed by the cell. Supporting this conclusion, susceptibility of tumor cells to proliferate was found, within the context of the present invention, to be a function of the amount of the shorter form of the MUC1 receptor.

To further support the conclusion that the MUC1* portion of the MUC1 receptor mediates cell growth, we performed the same antibody-induced dimerization experiment on MUC1-negative cells that had been transfected with a truncated MUC1* receptor terminated at the N-terminus of the PSMGFR sequence. FIG. 3 is a graph of a cell proliferation assay in which human embryonic kidney (HEK) 293 cells (MUC1-negative) that had been stably transfected with a MUC1 receptor that had a truncated ectodomain, terminated at the end of the PSMGFR sequence, were treated with anti-PSMGFR antibody. Normalized cell growth is plotted as a function of antibody concentration and shows that the MUC1* portion of the MUC1 receptor mediates cell growth via dimerization of this portion of the receptor.

To further demonstrate that the MUC1 cleavage product, MUC1* functions as a growth factor receptor, we generated an agent to bind to MUC1* and block its dimerization. The anti-PSMGFR antibody was proteolyzed and then purified over a MUC1* affinity column to produce the monovalent antibody that would bind to MUC1* but prevent its dimerization. The addition of the monovalent anti-MUC1* should not produce a bell-shaped curve, but rather should block the growth of MUC1* expressing cells. FIG. 4 is a graph of a cell proliferation assay in which three (3) cell lines were treated with the monovalent-anti-PSMGFR. The graph shows that the control cell lines (A) HeLa and (B) HEK 293s are unaffected by the addition of the antibody but in MUC1-positive breast cancer cell line 1504 (C) and (D), cell growth is inhibited.

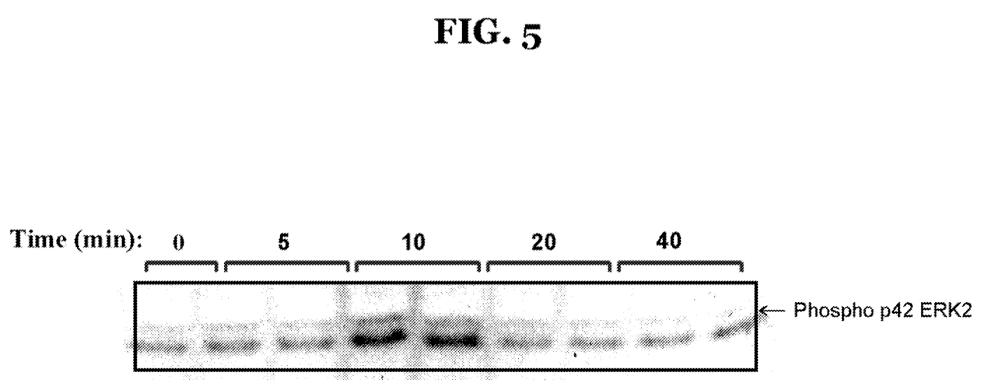

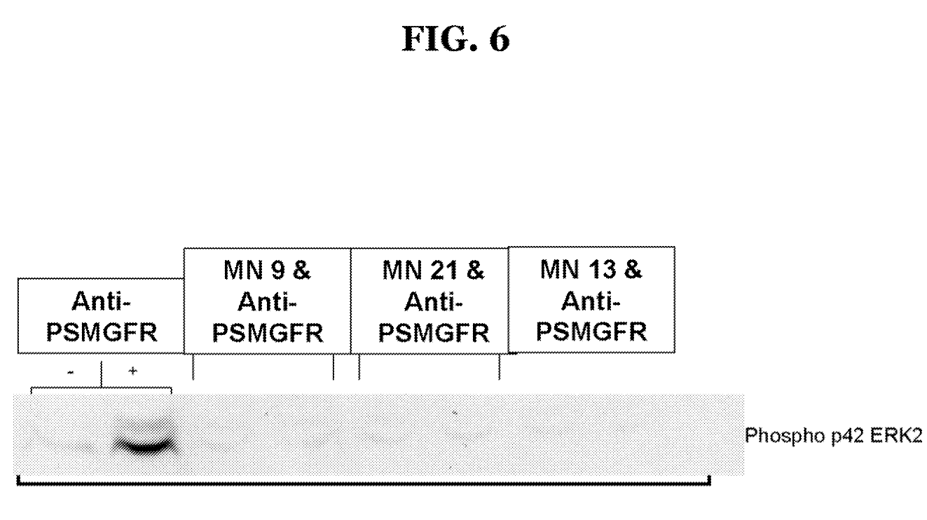

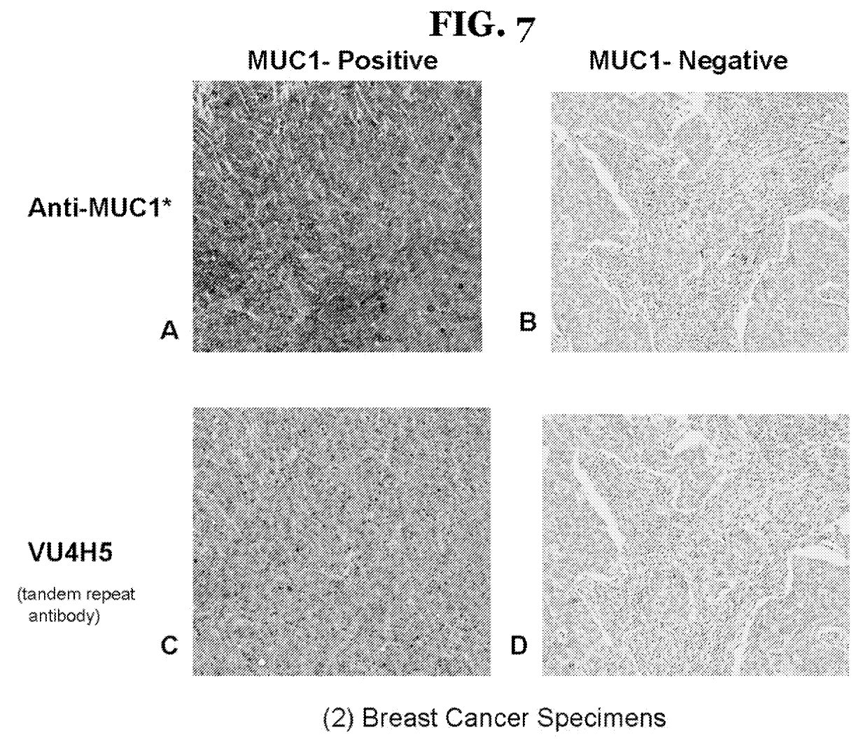

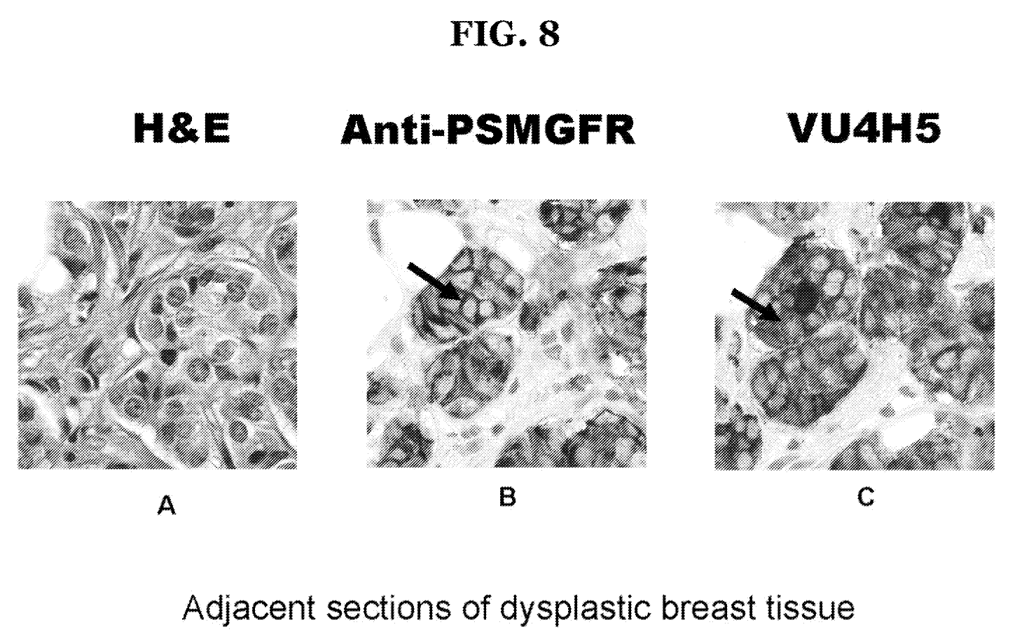

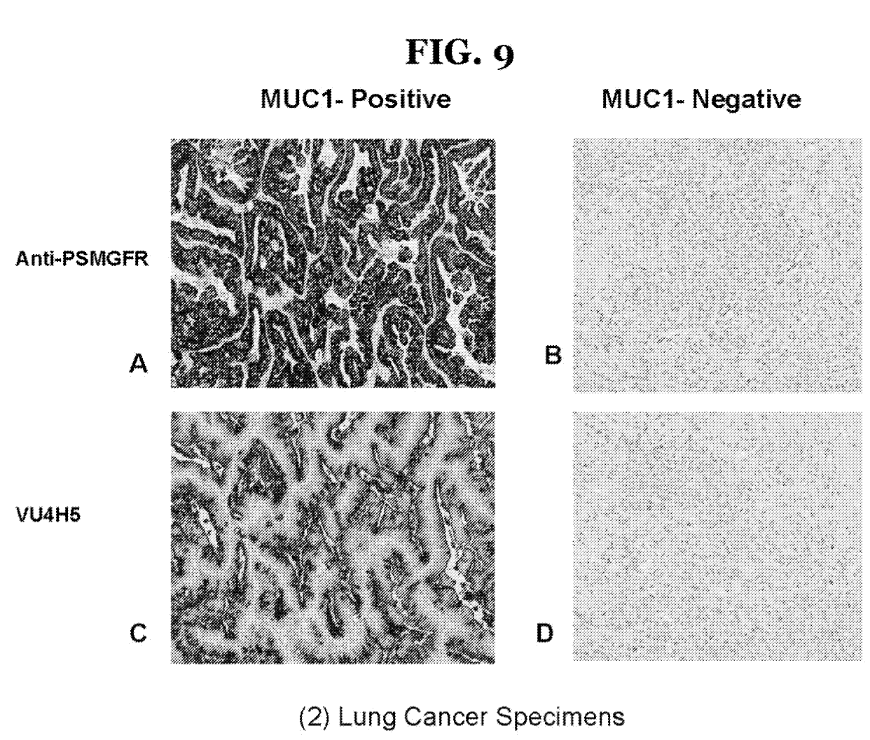

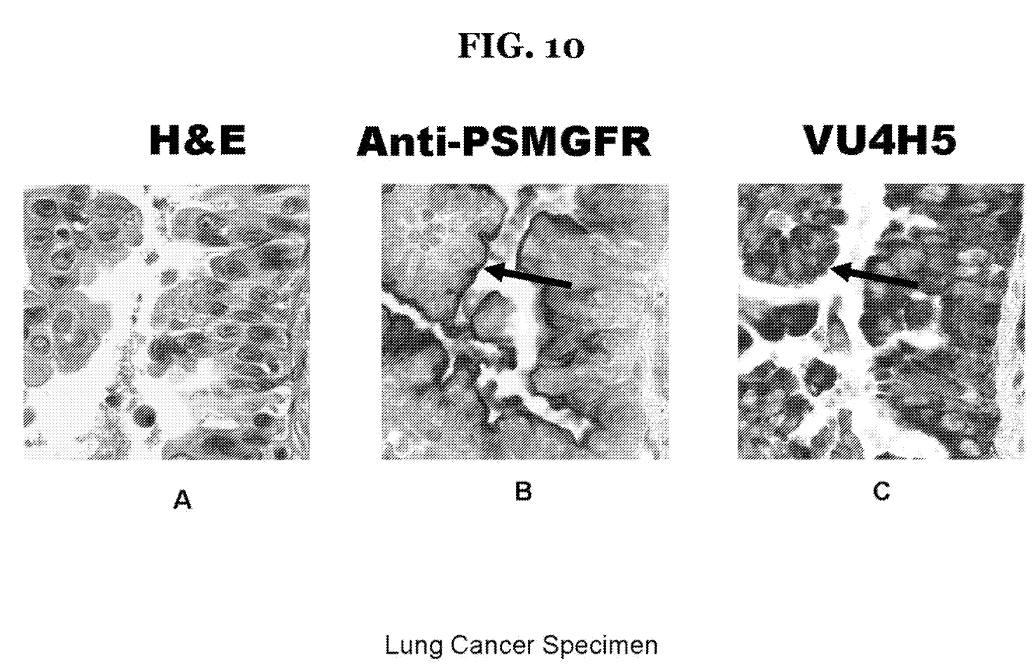

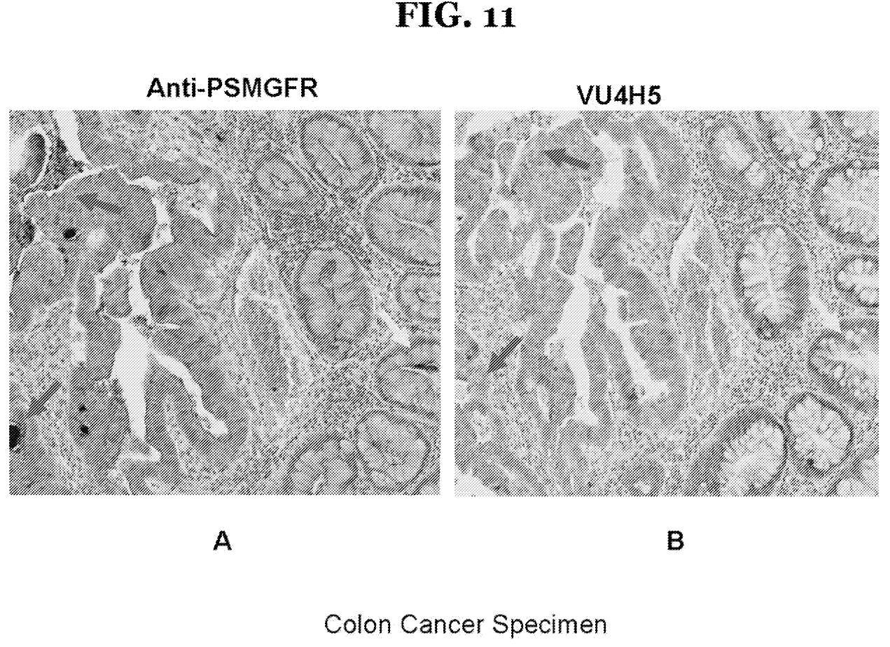

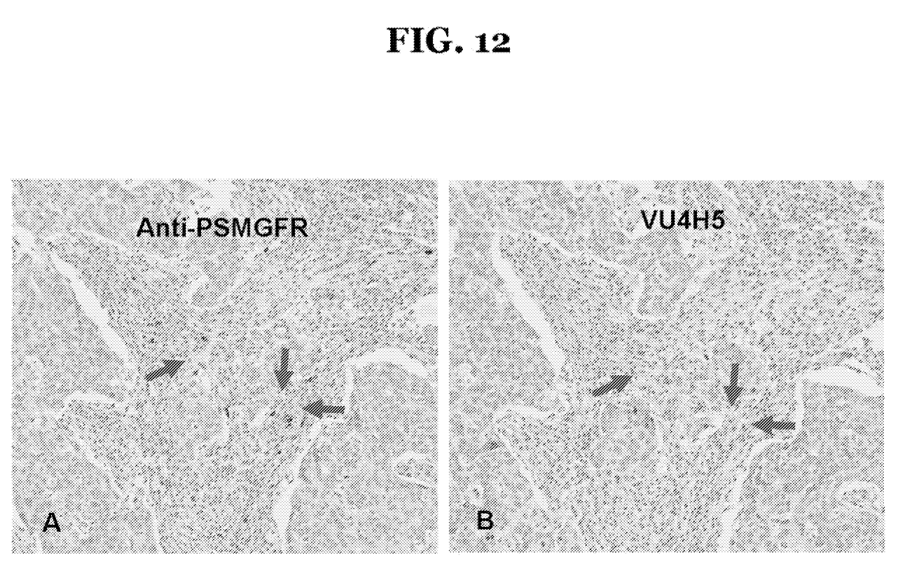

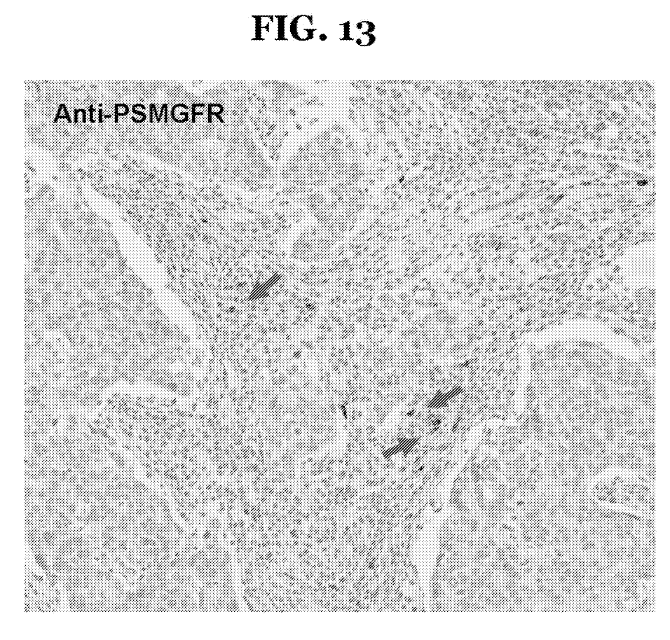

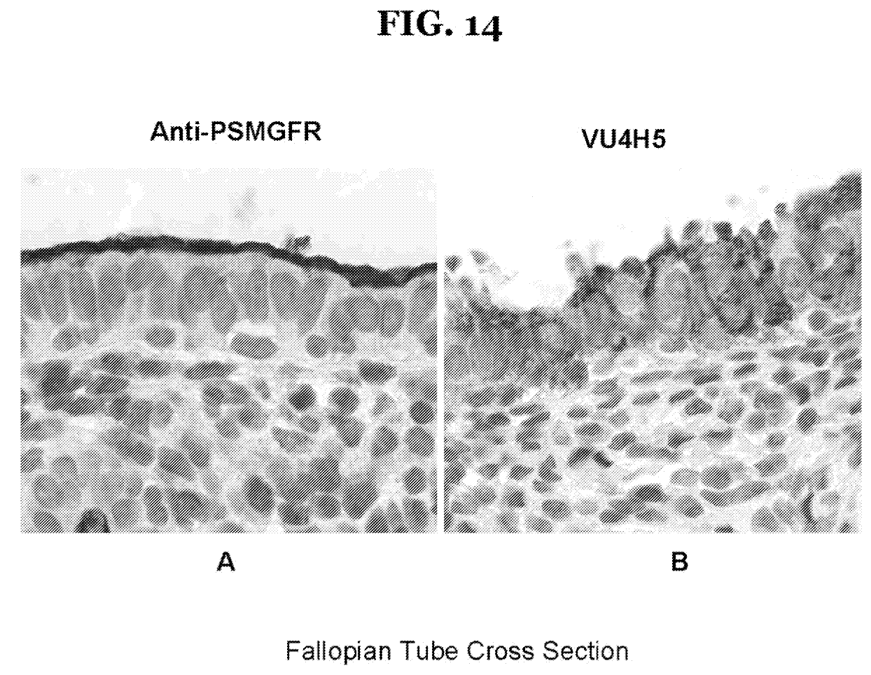

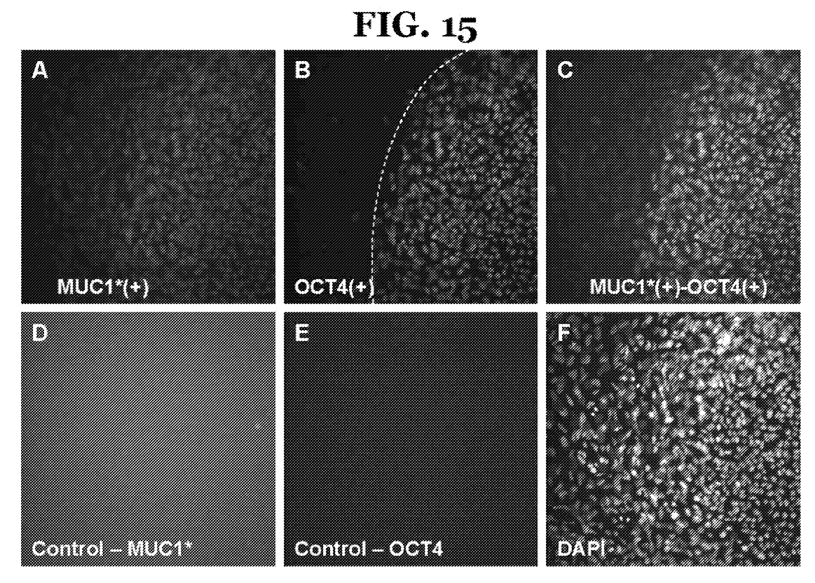

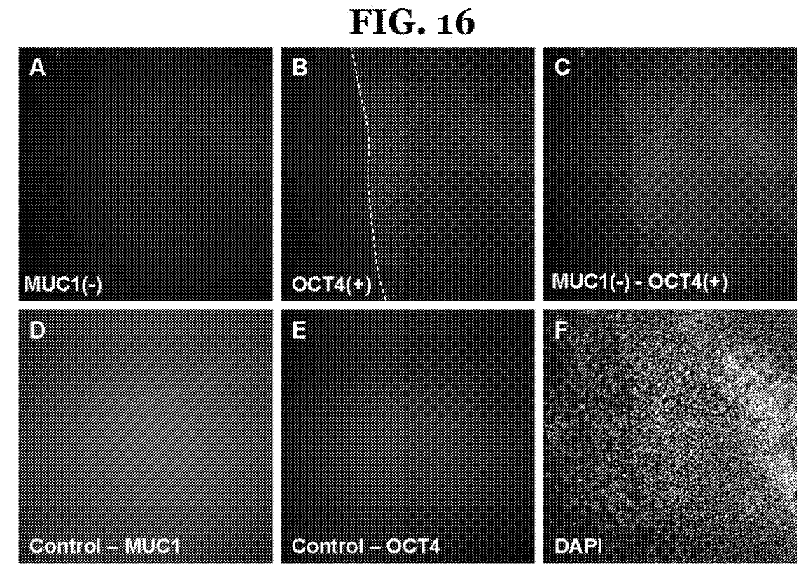

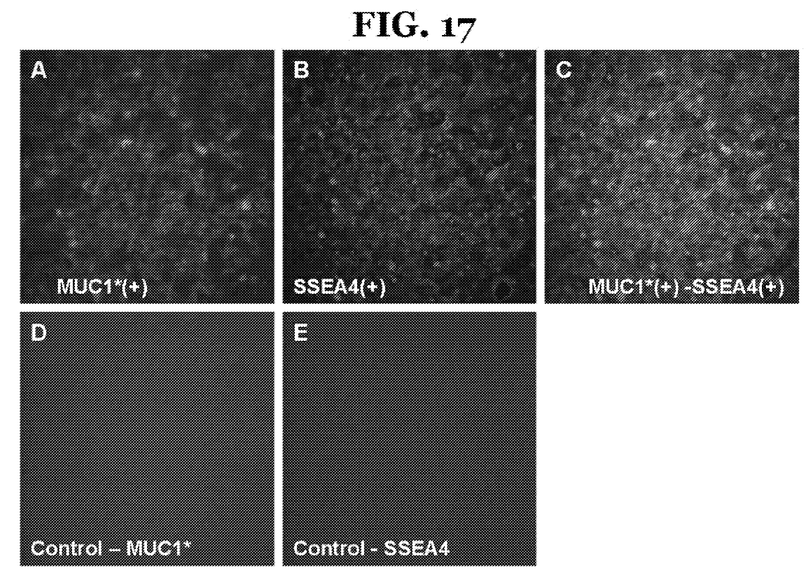

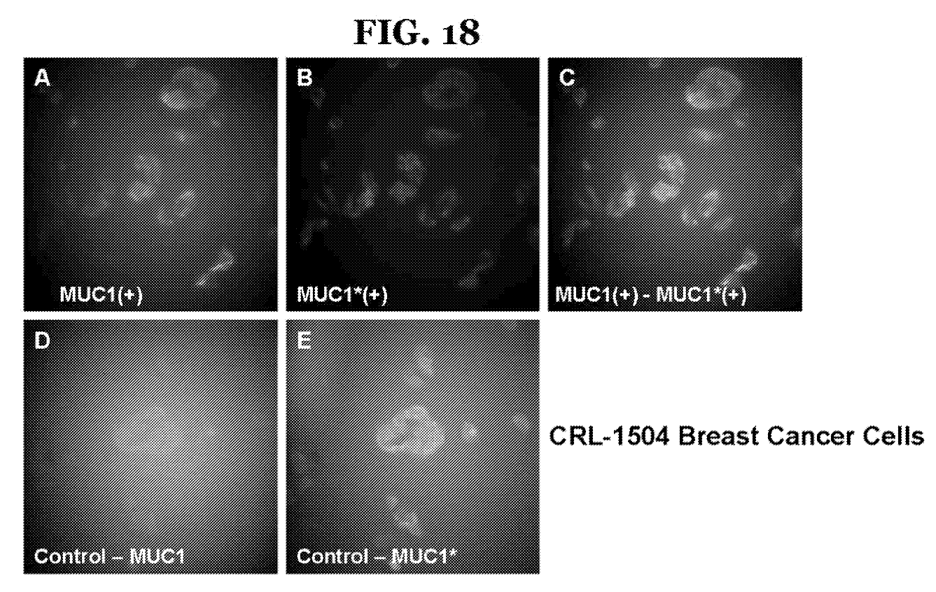

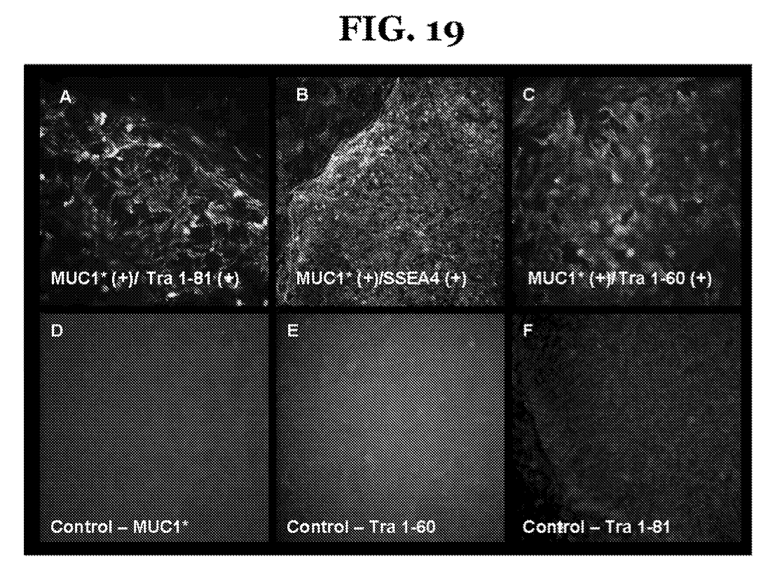

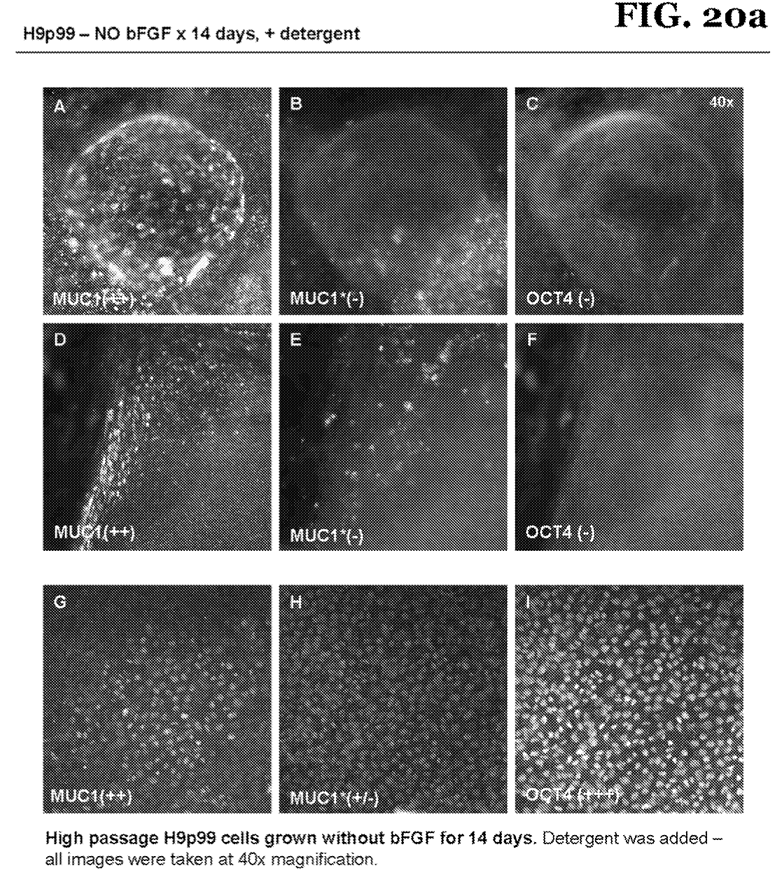

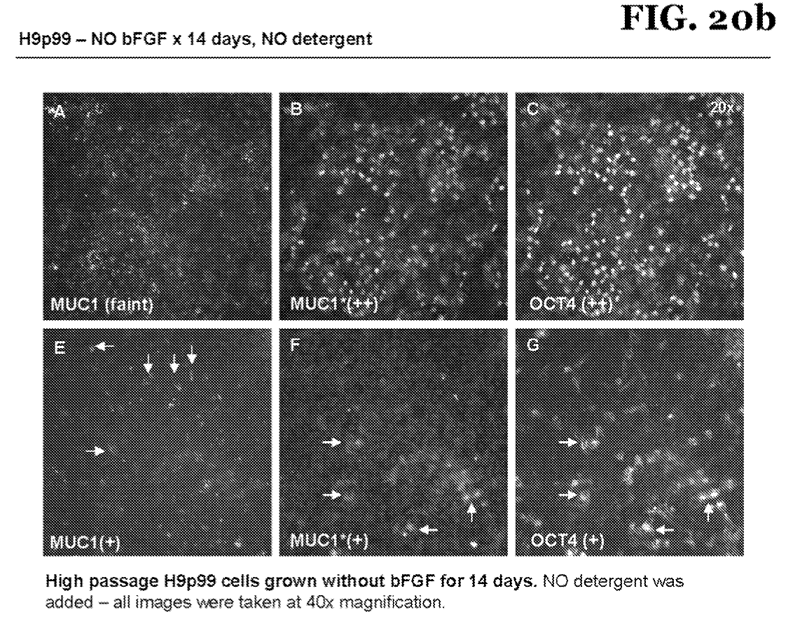

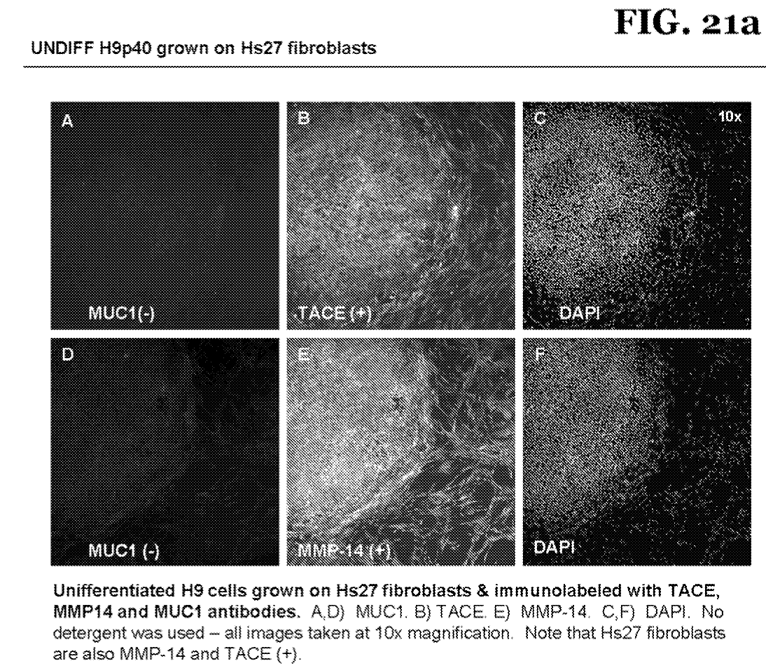

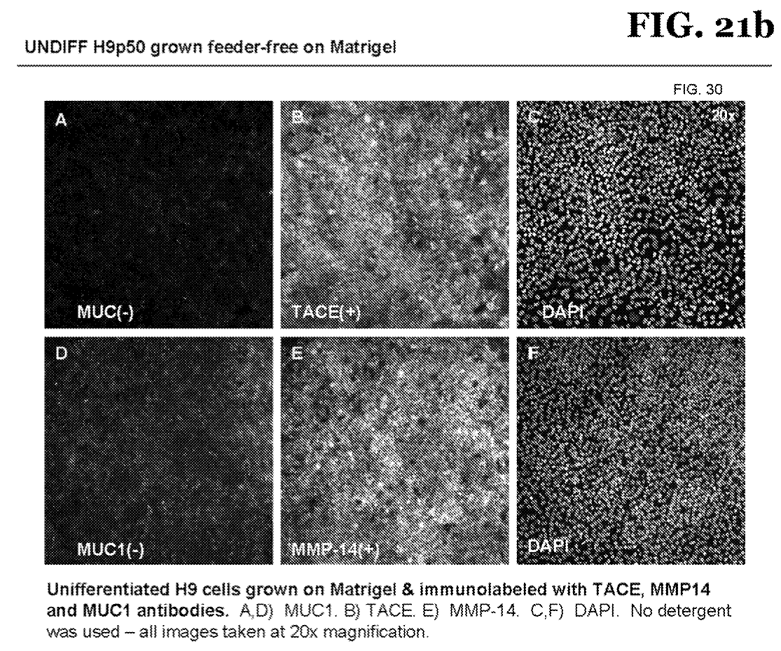

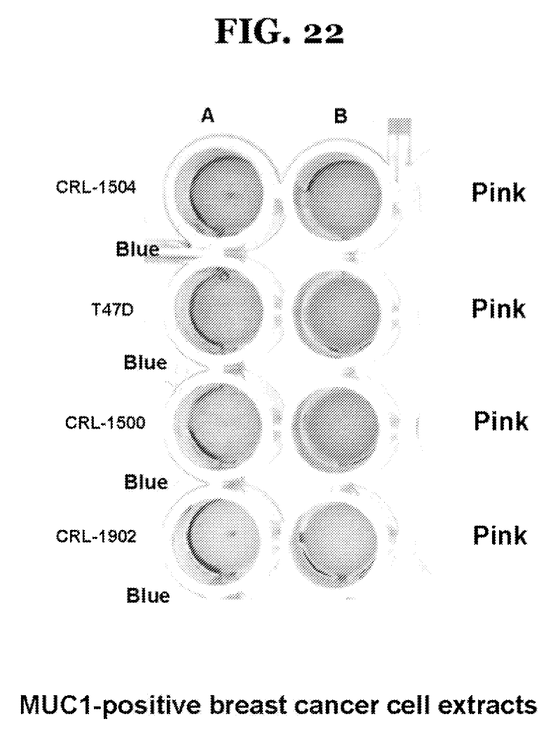

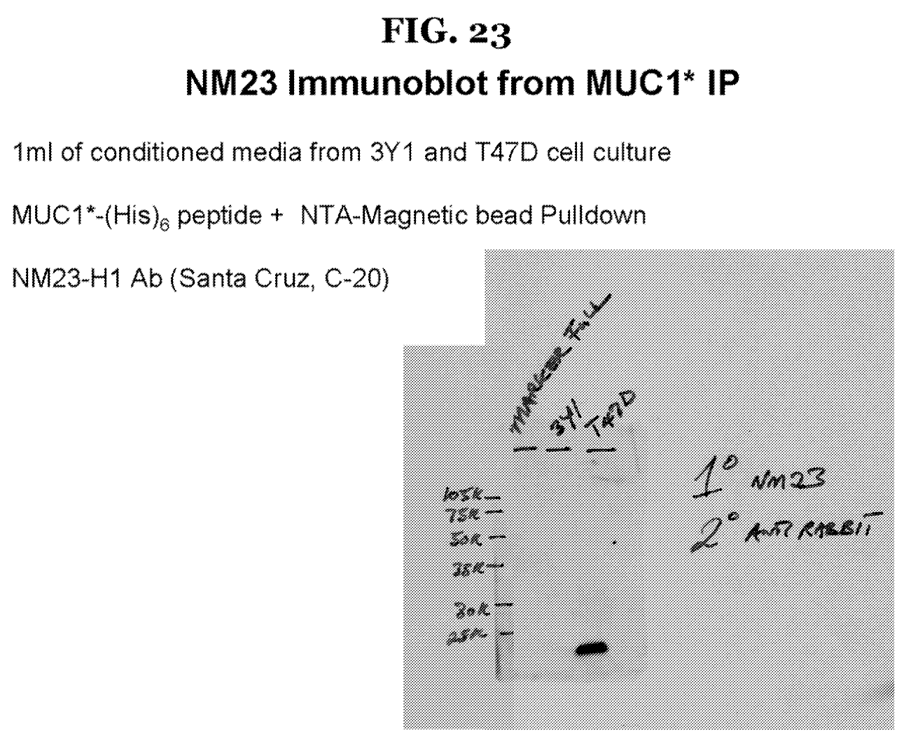

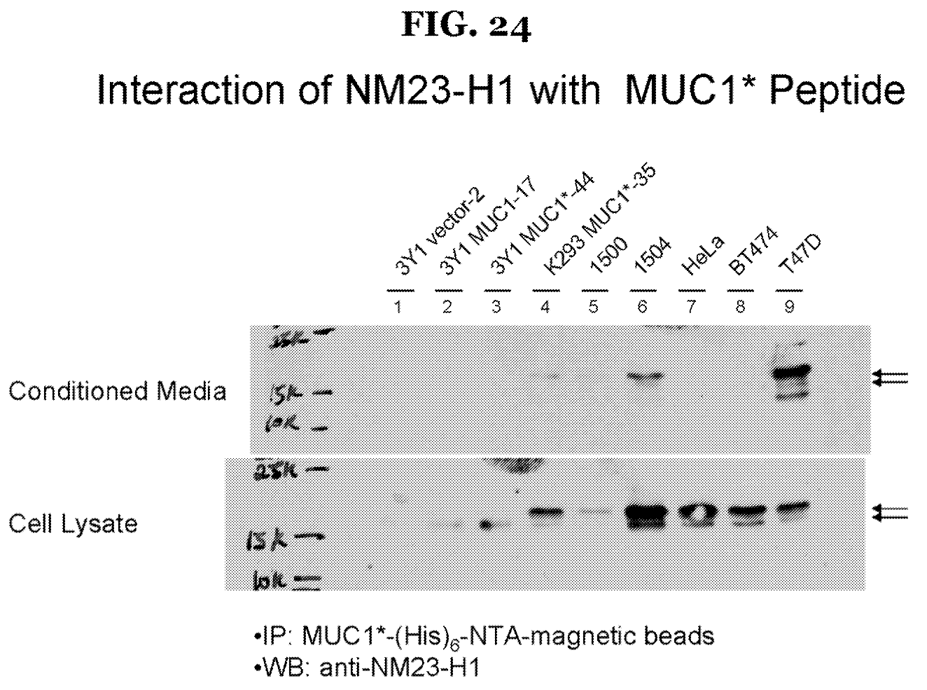

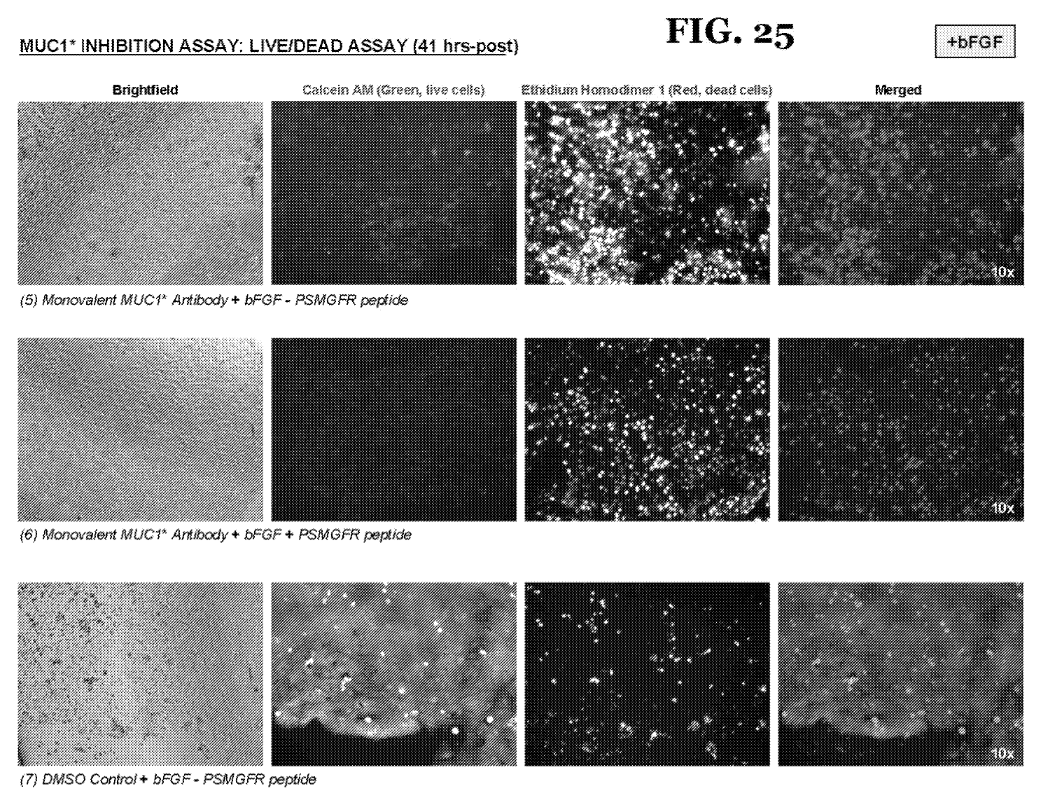

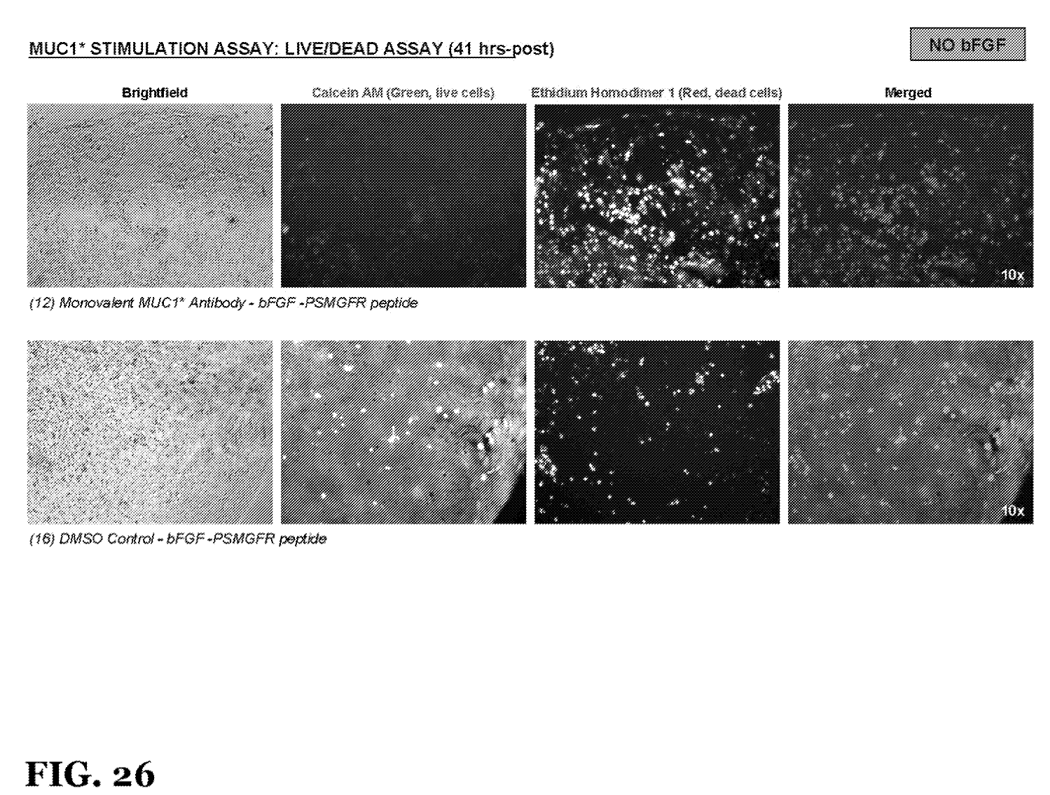

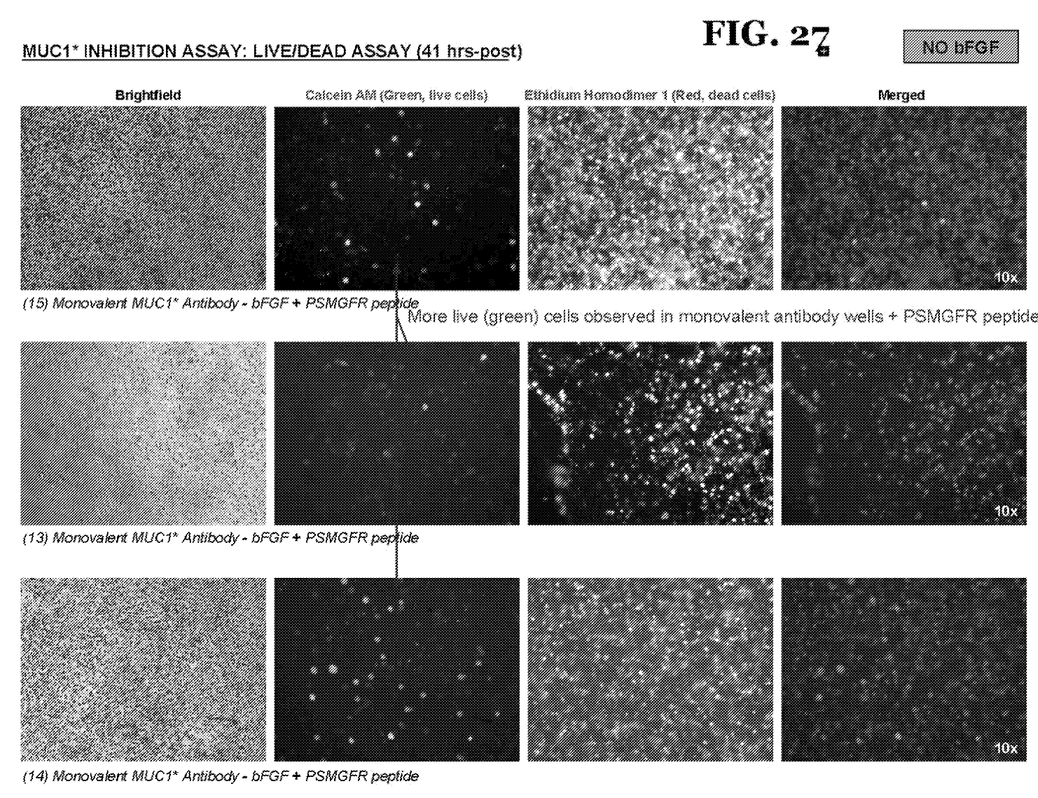

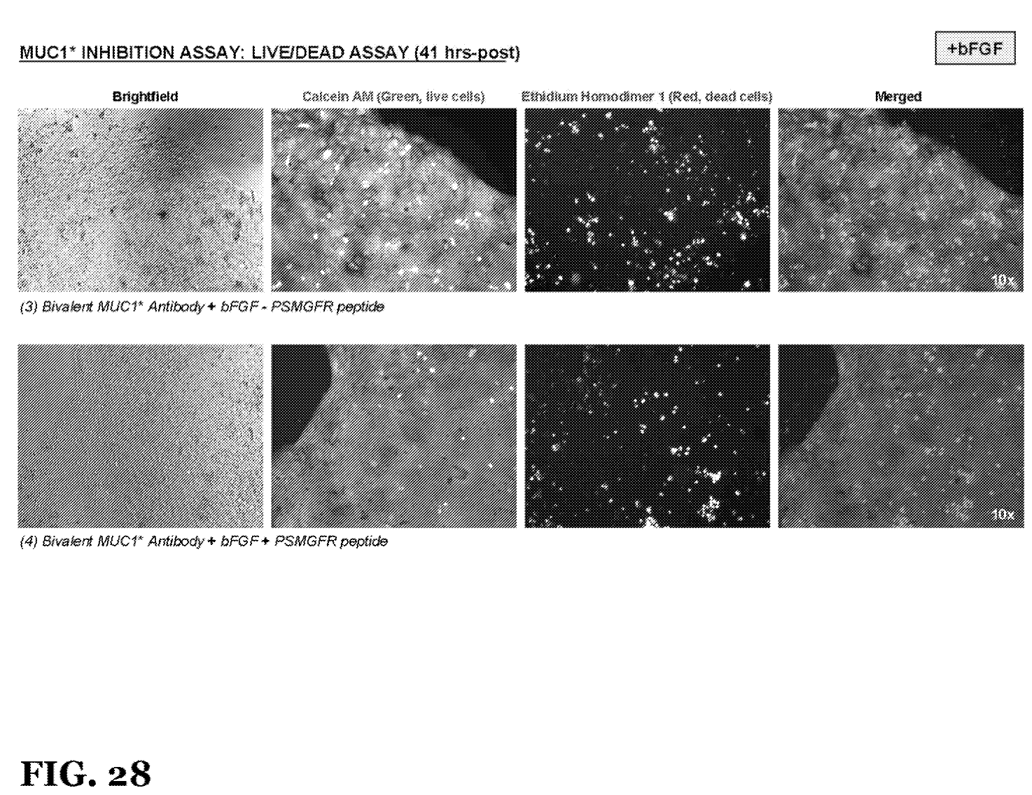

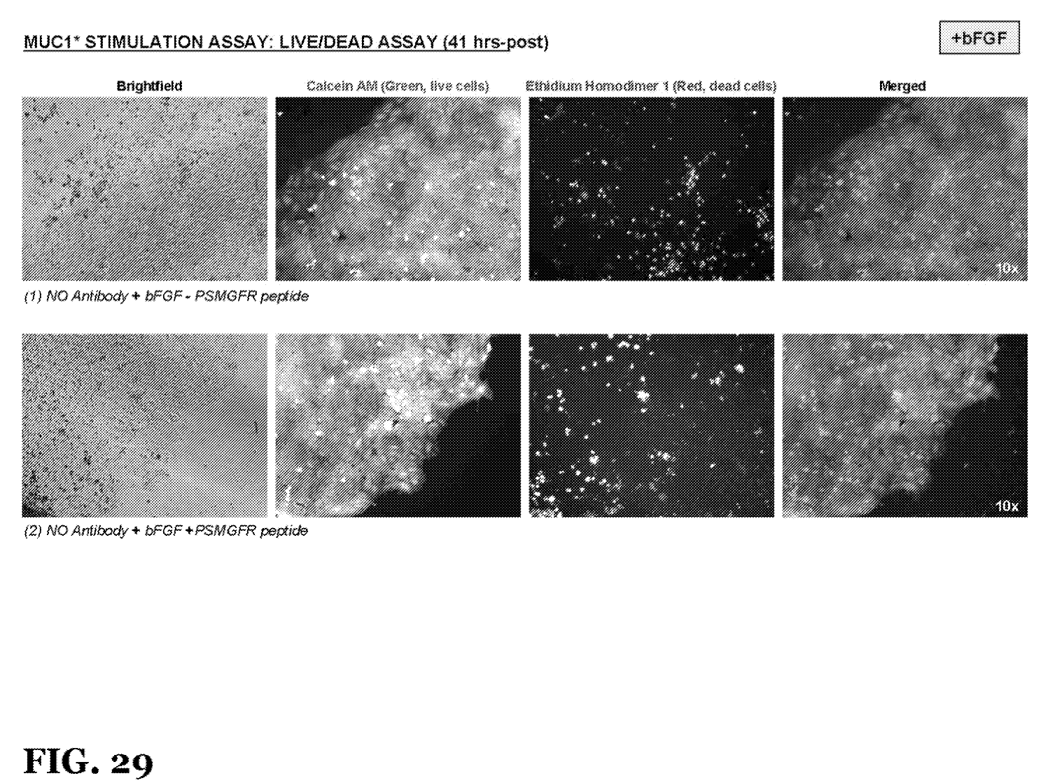

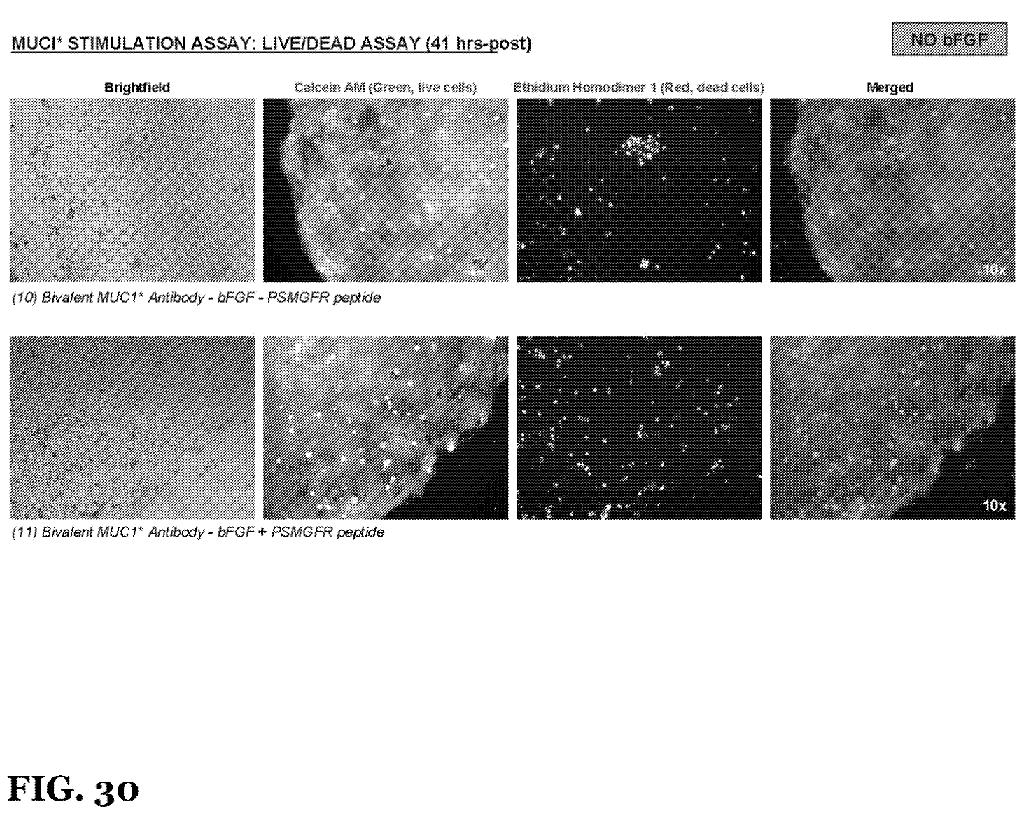

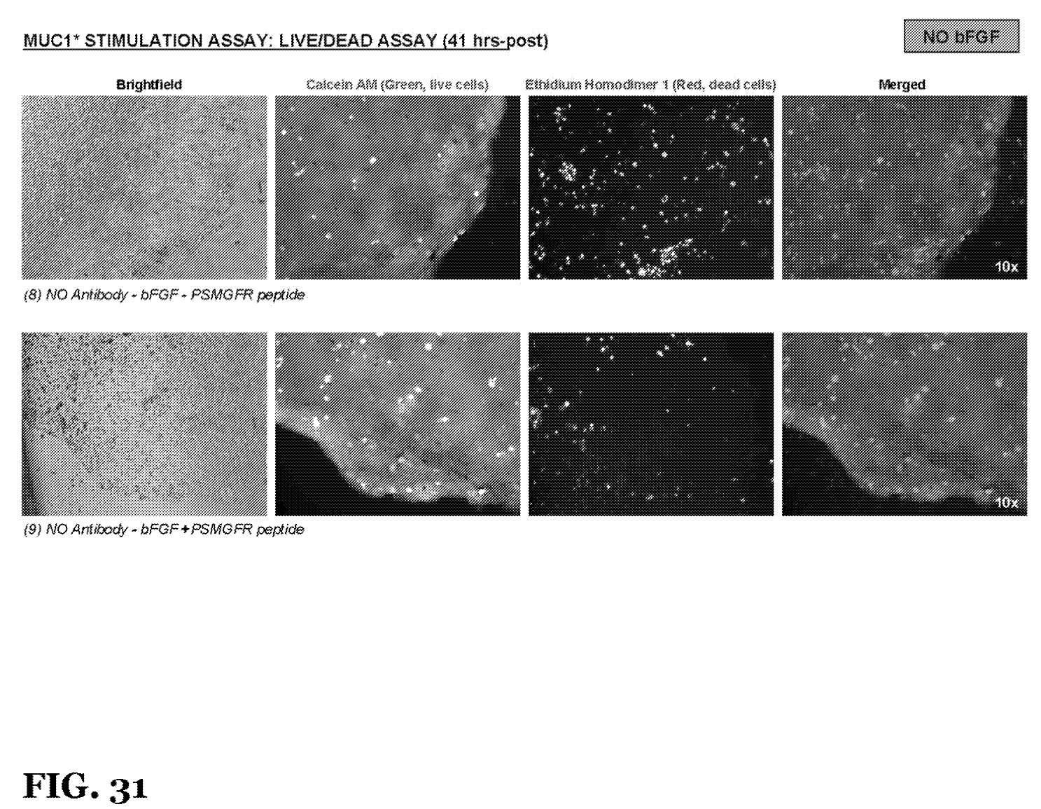

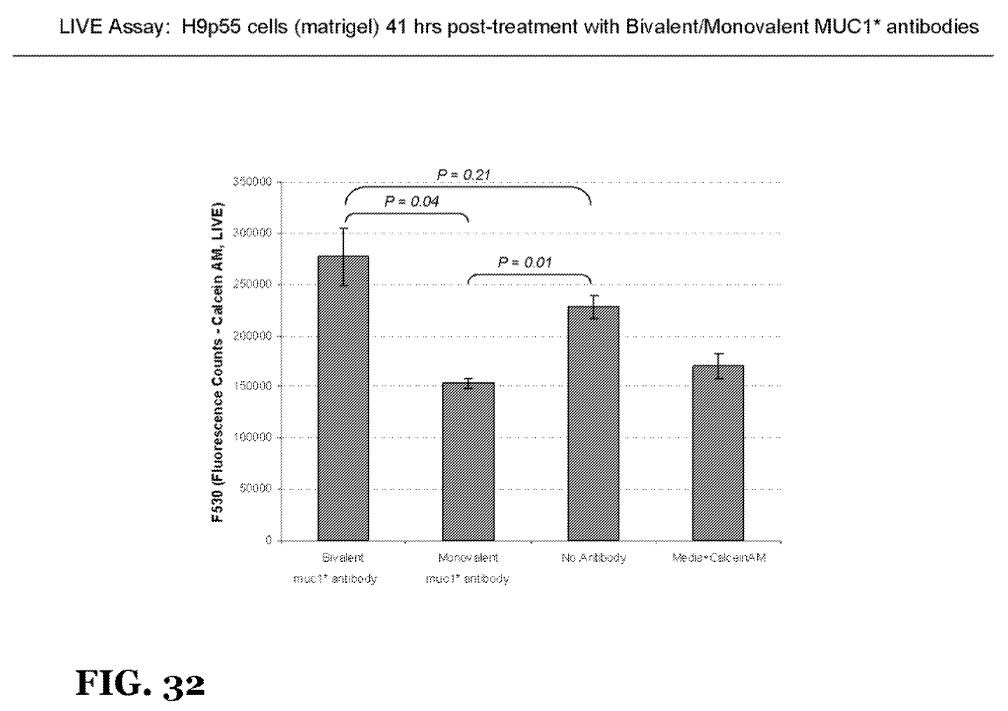

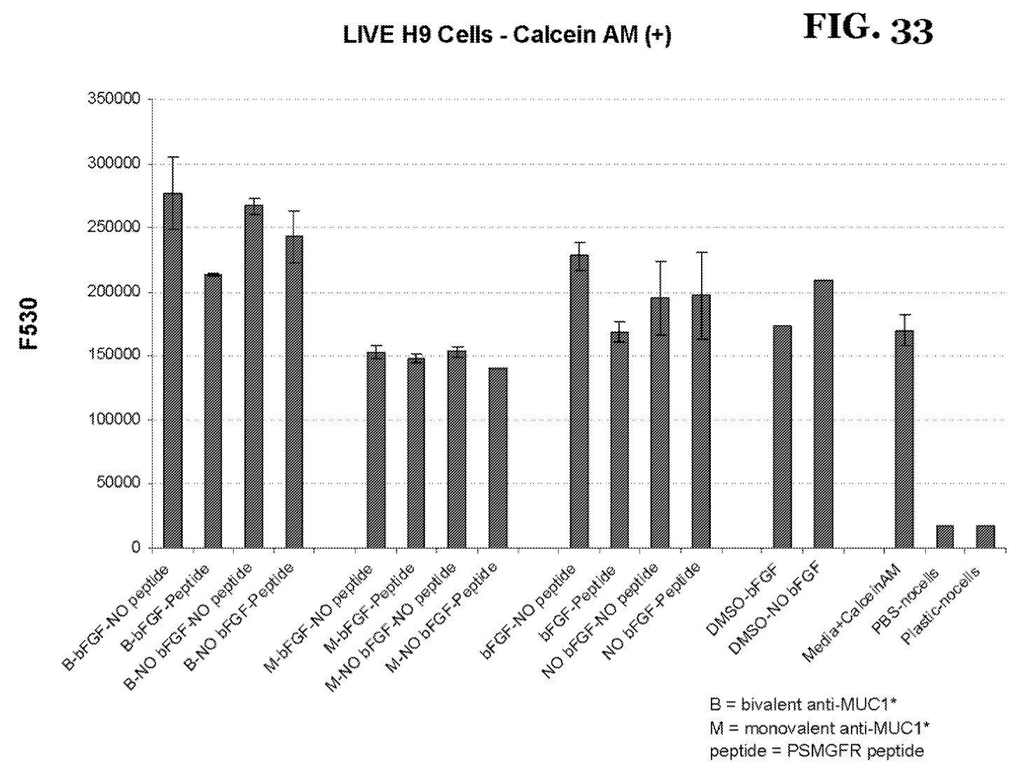

We further showed that when the MUC1* portion of the receptor is dimerized, the MAP kinase signaling pathway is activated, which commits the cells to divide. FIG. 5 is a western blot that shows that the ERK2 branch of MAP kinase signaling pathway is activated (ERK2 is phosphorylated) upon dimerization of MUC1*. FIG. 5 shows that within 10 minutes of the addition of bivalent anti-PSMGFR, ERK2 becomes phosphorylated. The western blot probe antibody is phospho-ERK2. FIG. 6 is a western blot of a competition experiment in which small molecules that bind to the PSMGFR region of MUC1 compete with anti-PSMGFR for binding to the site. In the presence of the competitor small molecule, the antibody does not bind and ERK2 phosphorylation is inhibited. These results indicate that the PSMGFR portion of the MUC1 receptor mediates cell growth and dimerization of the receptor can trigger this growth signal.

These results support the conclusion that the portion of the MUC1 receptor that acts as a growth factor receptor is a cleavage product in which much or all of the IBR is released from the cell surface. Further, these results support the conclusion that tumors in which a good percentage of the MUC1 receptors have been cleaved to release the TPSIBR (SEQ ID NO:18) are especially aggressive cancers and those that are cleaved to release the entire IBR, leaving PSMGFR (SEQ ID NO:11) attached to the cell surface are even more aggressive. Therefore, antibodies that are raised against the TPSIBR (SEQ ID NO:18) portion of the MUC1 receptor can be used to assess the aggressiveness of cancers that are MUC1-positive.

MUC1 Expression in Stem Cells

MUC1* is on hESCs

The Applicant has discovered that a shortened form of the MUC1 receptor that we call MUC1* consisting essentially of the cytoplasmic tail and transmembrane portions of MUC1 but having an ectodomain that is terminated after the end of the PSMGFR sequence SEQ ID Nos:10 and 11), is expressed on the surface of undifferentiated, pluripotent human embryonic stem cells (hESCs). This truncated form of the MUC1 receptor stained positive when probed with an antibody raised against the PSMGFR but showed no staining when probed with the VU4H5 antibody that binds to the distal portion of the MUC1 receptor, which we previously showed is most often cleaved and released from the surface of cancer cells. Low passage number (40 passages) H9 human embryonic stem cells (Wicell, Madison, Wis.) stained positive when probed with a rabbit polyclonal antibody raised against a peptide of SEQ ID No:10, which corresponds to the first 56 amino acids of the extracellular domain of the human MUC1 receptor (anti-PSMGFR). Double staining showed that the same cells reacted positively with anti-PSMGFR and an antibody against OCT4, which is a known marker for human undifferentiated stem cells, see FIG. 15. Incubation of the same cells with an antibody against the tandem repeat portion of the MUC1 receptor, VU4H5, did not produce surface staining or cytoplasmic staining. See FIG. 16. The addition of a detergent to the VU4H5 mixture to permeabilize the cell membrane also failed to produce staining. FIG. 17 is also an immunocytochemical image in which the same hESCs were double stained with anti-PSMGFR (reacts with the truncated form of MUC1) and SSEA4 which is another recognized marker for stem cell pluripotency or undifferentiation. MUC1* is expressed on the surface of the H9 hESCs as evidenced by the fact that staining with anti-PSMGFR was clear and robust without needing to add detergent, which is required to visualize proteins in the cytoplasm and/or in the nucleus. Also, as can be seen in FIG. 19, staining with the anti-PSMGFR antibody, also referred to herein as anti-MUC1*, produced the “chicken wire” effect that is characteristic of cell surface proteins. Double staining experiments also showed that the MUC1* receptor co-localized with the pluripotent markers SSEA4, TRA 1-60 and TRA 1-81. See FIG. 19.

Cleavage Enzymes MMP14 and Adam 17 Cleave MUC1 in Cancer Cells and on Stem Cells

MMP14 and ADAM17 exist on the surface of the same stem cells that express MUC1*, OCT4 and the other markers for pluripotency. This leads to the conclusion that on hESCs, the MUC1 receptor is cleaved to leave MUC1* on the cell surface and this cleavage is carried out by the membrane matrix metalloprotease MMP14 (also called MT1-MMP and ADAM17). This underscores another similarity between cancer cells and stem cells; MMP14 and ADAM17 have both been implicated in the cleavage of the MUC1 receptor on cancer cells.

Differentiated stem cells express the full-length receptor and not the cleaved form, MUC1*

It is within the purview of the present invention in which RNAi is used to stimulate growth of stem cells by suppressing natural inhibitors of MMPs, such as TIMPS. Since MMP-14 and TACE are required to cleave MUC1 to the growth factor receptor form that stimulates stem cell growth, the inhibition of these cleavage enzymes inhibits MUC1 cleavage. We have observed that the cessation of MUC1 cleavage on stem cells signals the onset of differentiation. Therefore, it follows that agents that promote MUC1 cleavage are useful agents for stimulating stem cell growth.

Conversely, RNAi that suppresses enzymes such as MMP-14 and TACE that cleave MUC1 are beneficial agents that is used to induce differentiation of stem cells to inhibit the growth of cancer cells and particularly of cancer stem cells. In certain instances, it is beneficial to target RNAi to particular cells or tissues. For example, to induce differentiation one delivers interference RNA that suppresses MUC1 cleavage enzymes to the targeted cells by attaching both RNAi and a targeting antibody to the same entity. In one embodiment, the common entity that presents both the targeting antibody and the siRNA or shRNA is a nanoparticle. In a preferred embodiment, the nanoparticle is gold and coated with a self-assembled monolayer (SAM). In a still more preferred embodiment, the nanoparticle bears anti-SSEA4, anti-TRa 1-81 or anti-Tra 1-60 and an interference RNA that suppresses MMP-14 and or TACE expression.

FIGS. 20a and 20b are images of undifferentiated and thus pluripotent human embryonic stem cells grown over either Hs 27 human foreskin fibroblast feeder cells (a) or grown over matrigel (b). In both cases, we observed that undifferentiated stem cells stained robustly positive for MMP-14 and TACE while no full-length MUC1 is detectable. These results indicate that MMP-14 and TACE cleave full-length MUC1 and that cleavage does not require agents secreted by fibroblast feeder cells. These stem cells were deemed to be undifferentiated by visual inspection and because they stained positive for OCT4 and other markers of undifferentiated state such as SSEA4, Tra 1-81 and Tra 1-60. In sharp contrast, when cells were allowed to differentiate or forced to differentiate by withholding the addition of exogenous bFGF for 14 days and greater, experiments showed that stem cells were coated with full-length MUC1 protein, had little or no MMP-14 or TACE expression and little or no cleaved MUC1 which we refer to herein as MGFR or MUC1*. In addition, these cells no longer expressed OCT4.

We observed that cells that had undergone differentiation, expressed full-length MUC1 by virtue of the fact that they reacted with the VU4H5 antibody that binds to the tandem repeat units. They no longer stained positive for the presence of MUC1*. Undifferentiated cells that stain positive for OCT4, stain negative for full-length MUC1. However, once the cells have differentiated, the OCT4 staining disappears and they stain positive for the presence of the full-length receptor. Note however, that along the interface between undifferentiated and differentiated, these cells stain positive for both MUC1 full length and OCT4. This result calls into question whether or not staining positive for OCT4 is a true indicator of undifferentiated stem cells. According to these experiments, using the known stem cell pluri-potency markers is not sufficient to identify truly pluri-potent, undifferentiated stem cells. Rather, one must include antibodies against MUC1*. Accurate identification of pluripotent stem cells is therefore based on staining positive for MUC1* and OCT4 but negative for full-length MUC1 on the cell surface. SSEA4, Tra 1-60 and Tra 1-81 may be used in conjunction with antibodies against MUC1* and OCT4 to more accurately identify and select undifferentiated stem cells.

Further, H9 cells that were grown for 21 days without the addition of bFGF to induce the onset of differentiation, showed surface staining for both MUC1* and for full length MUC1 (FIG. 20). This pattern of surface staining is similar to what is observed when cancer cells are double stained for MUC1* and for full-length MUC1. Both are expressed on the surface of cancer cells, see FIG. 18.

Higher passage number stem cells are not truly undifferentiated even though they test positive for the markers of pluripotency according to the state of the art. The staining of higher passage number stem cells (passage 84), which were thought to still be pluripotent, differed in their antibody staining patterns with respect to MUC1 and MUC1* staining. In contrast to the staining of the low passage number cells, these cells stained positive for full length MUC1 in the cytoplasm. The staining levels and patterns of MUC1*, SSEA4, TRA 1-60, TRA 1-81 and OCT4 remained the same as was observed for the low passage number stem cells.

Taken together, these results indicate that the state of the art markers for identifying pluripotent stem cells are inadequate. Antibodies that assess the cleavage state of the MUC1 receptor and/or determine whether it is on the surface or in the cytoplasm must be integrated to accurately identify truly pluripotent stem cells.

Since we know that the cleaved form of the MUC1 receptor, MUC1*, is expressed on stem cells and cancer cells, and since we know that in this form, the receptor functions as a growth factor receptor that drives the growth of these cells, it follows that the MUC1* receptor is on and drives the growth of progenitor cells, cells that may yet undergo another step of differentiation, and even on cells that rapidly turnover. Neutrophils, for example exclusively express the proteolyzed form of MUC1 on their cell surface and not the shed portion that contains the tandem repeats. The luminal surface of ducts, such as breast ducts, prostate ducts, and colon ducts are lined with MUC1*.

These rapid turnover cells have a high level of MUC1 in their cytoplasm. FIG. 14 shows a tissue specimen of a healthy fallopian tube that shows that the luminal cells that line the tube stain positive on the cell surface when probed with anti-PSMGFR (A), but only stain positive in the cytoplasm when probed with VU4H5 (B). The cells that line the fallopian tubes and other ducts display a MUC1 cleavage product that contains the PSMGFR region but not the tandem repeats. These luminal cells are not cancerous but must be frequently replenished. These tissues contain stem cells and progenitor cells to make this rapid turnover of cells possible.

In light of these findings, the predominant form of the MUC1 receptor on cells that rapidly divide such as neutrophils, their pre-cursors, stem cells, including pluri-potent stem cells, and the like, is the cleaved form, comprising essentially of PSMGFR. This cleaved MUC1 mediates proliferation and expansion of some if not all stem cells, progenitor cells, neutrophils, pre-cursors and other rapidly dividing cells.

Closer scrutiny of the newly differentiating colonies revealed that during the initial stages of cellular differentiation, there were rare transition areas in which cells simultaneously presented markers for the undifferentiated state and markers for the differentiated state. It is significant to note that cells in these transition areas stain positive for full-length MUC1 and OCT4, shown in FIGS. 21a and 21b. These results lead to the conclusion that detecting the presence of OCT4 is insufficient for determining whether a cell is undifferentiated or differentiating. The cessation of cleavage of full-length MUC1 is the first sign of the initiation of differentiation. Therefore to identify undifferentiated stem cells, which are also the pluripotent stem cells, one must detect the presence of MGFR and the absence of full-length MUC1. Methods for the identification and selection of pluripotent and/or undifferentiated stem cells may also optionally include selecting cells that express NM23, MMP-14, TACE and OCT4. For accurate identification and selection of pluripotent stem cells, one may also wish to include a negative selection criterion which should be the absence of full-length MUC1 and may optionally include other markers of differentiation of germ line markers.

Identification and Isolation of Stem Cells

Therefore, to identify stem cells and stem cell sources, one must integrate methods for determining which form of MUC1 is on the cell surface and whether or not full-length MUC1 can be detected on the surface or in the cytoplasm. Truly pluripotent stem cells are characterized by MUC1* surface expression, lack of full-length MUC1 staining, and the presence of OCT4 in the nucleus; surface markers TRA 1-60, TRA 1-81 and sometimes SSEA4 may also be expressed on cells in these pluripotent colonies.

In a preferred embodiment, true pluripotent stem cells are identified and/or isolated by selecting those cells that express MUC1*, SSEA4, TRA 1-60, and TRA 1-81 on the cell surface, OCT4 in the nucleus and do not have significant surface expression of full length MUC1. In a yet more preferred embodiment, pluripotent stem cells are identified and/or isolated by selecting cells that show surface expression of MUC1*, OCT4 in the nucleus, and do not exhibit surface expression of full-length MUC1.

The invention includes using these markers, including antibodies against MUC1* and MUC1 full-length, to identify new stem cell sources. To identify sources of adult stem cells and isolate stem cells therefrom, sources that would be screened include but are not limited to tissues, fluids, breast milk, fat, hair folicules, mucous and mucosal lining, bone, bone marrow, blood, brain cells, eye and the like. To identify sources of embryonic stem cells and isolate stem cells therefrom, sources that would be screened include but are not limited to embryonic tissues, fluids, amniotic fluid, placental blood, placenta, umbilical cord blood and tissue, blood, blastocysts, fetus, fertilized egg and the like.

A salient feature of the invention is that cells that range from totipotent stem cells, to pluripotent stem cells, to multipotent stem cells, to rapidly dividing fully differentiated cells, are characterized by the ratio of MUC1* to full length MUC1 on the cell surface, wherein this ratio is inversely proportional to the degree of differentiation. For example, pluripotent stem cells may be identified by virtue of the fact that the ratio of cleaved to uncleaved MUC1 on the surface is essentially infinite as there is no detectable full-length receptor on the surface of these cells. However, as cells differentiate, the cleavage of MUC1 decreases so that the ratio of MUC1* to MUC1 full-length decreases and approaches zero when the cells are fully differentiated.

There may be cases in which it is desirable to identify and isolate a population of cells that have already undergone some differentiation, such as differentiation steps that commit those cells to differentiate along one of the three germ lines: endoderm (gland, organs, and digestive track), ectoderm (skin, nails, hair, eye, ear, tooth enamel, pituitary gland, mammary gland, nervous system), or mesoderm (muscle, bone, lymphatic tissue, spleen, blood system stem cells, heart, lungs, reproductive). If it is desired to have a population of stem cells all differentiate into skin cells, then it may be desirable to start with cells that have already begun to commit to that pathway, which would be accomplished by selecting cells that express MUC1* on their surface and also some of the early markers for commitment to that germline. For example, markers for the endoderm germline include GATA 4/6, HNF families, FGF5, Wnt3, EndoA, collagen IV, and t-PA; markers for the ectoderm include ECTO-V, LpC2 actin and LpS1 mRNA; markers for the mesoderm germline include NESTIN, MAP-2, GATA4, TWIST and Tbx20 and ab20680. Antibodies against subsets of these markers are used in combination with anti-PSMGFR to identify multi-potent stem cells that have begun to commit to a specified fate. The ratio of MUC1* to full-length MUC1 shifts from high, (MUC1* expression is high and full-length MUC1 is undetectable) to low (MUC1* is undetectable) as cells go from pluri-potent to multi-potent to fully differentiated.

Cancer Stem Cell Identification and Isolation

It has recently been established that there are cancer stem cells as defined by the ability to confer disease in a host with the introduction of small numbers of cancer cells on the order of tens or hundreds rather than the typical requirement for millions of cells. There are of course many therapeutic reasons for wanting to detect cancer stem cells in a variety of circumstances. The art of identifying cancer stem cells is in its infancy. Current research shows that cancer stem cells are characterized by a set of markers that includes CD133, CD44 and ESA, in particular CD44 (high) and CD24 (low). Recall that we have demonstrated that MUC1* is highly expressed on the surface of cancer cells and that a high level of surface expression of MUC1*, together with a low level of surface expression of full-length MUC1, indicates aggressive cancer cell growth that is also resistant to treatment with chemotherapy agents. Therefore, an improvement in the current method for identifying cancer stem cells is to select cells that express known cancer markers, such as CD133, CD44 and ESA, and in particular CD44 (high) and CD24 (low), but also must display high levels of surface staining of MUC1* and low or no staining of the repeat portions of the MUC1 receptor.

Methods of the invention for identifying and isolating cancer cells and cancer stem cells CSCs are employed in a variety of circumstances. For example, CSCs are identified and removed from a patient as a part of a therapeutic regimen. Since CSCs can be in circulation, it would be advantageous to use methods of the invention to cleanse a patient's bodily fluids and other substances, including blood, bone, and bone marrow of CSCs before subsequent re-introduction into the patient or into another patient. Re-introduction of cells may take place after several types of therapeutic interventions to alleviate the cancer, which may include chemotherapy and radiation therapy. In one embodiment, methods of the invention are used to identify, isolate and remove cancer stem cells from a patient's blood as an anti-cancer therapy, a cancer preventative or cancer recurrence preventative. The patient's blood is removed from the patient and passed through an instrument that has a chamber that presents antibodies against MUC1* and a panel of other CSC markers such as CD144, CD44, and/or ESA. As MUC1*-positive cells pass through this portion of the instrument, they are captured and retained within the instrument while the sanitized blood is re-introduced into the patient. Methods of the invention are used for identification and isolation of cancer stem cells and subsequent removal of stem cells or cancer stem cells from a patient, for example to sanitize (to ensure the removal of all cancer stem cells from a patient) a patient's blood or bone marrow after cancer surgery or treatment.

The invention further anticipates using methods described herein to identify and isolate cancer stem cells from a variety of sources, for the purpose of ridding a person, animal or person/animal derived materials of cancer stem cells. Instances in which it is desired to rid biological materials of cancer and CSCs include those cases wherein material is destined for transplant or transfusion. Materials and/or sources from which such cancer stem cells may be identified and/or isolated from include but are not limited to a cancer patient, person suspected of having cancer, a person living or deceased, donating or being considered for receiving transplant substances including organs, cornea, tissue, skin, bone, bone marrow, and the like. Substances destined to be introduced into live tissues, organs or persons by transplantation, transfusion, injection, or oral administration, wherein it would be desirable to rid the substances of cancer or cancer stem cells include without limitation blood, organs, tissues, skin, fat, stem cells, bone marrow and cartilage.

Cell Expansion Through MUC1 Receptor Manipulation

The inventor previously disclosed that one could stimulate MUC1-positive cancer cells to grow by dimerizing the receptor. Many instances were illustrated using antibodies directed against the MUC1* portion since this portion of the receptor is the major MUC1 species on cancer cells. The inventor also showed that cell death is induced when MUC1 receptors are prevented from dimerizing. The inventor demonstrated that cell death or growth inhibition results when MUC1-positive cancer cells, or cells transfected with MUC1* are incubated with the monovalent anti-PSMGFR antibody or FAb.

Herein, the inventor discloses for the first time that human embryonic stem cell growth can be manipulated by causing MUC1* dimerization or by preventing dimerization. In summary, embryonic stem cells are stimulated to grow when the MUC1* portion of the MUC1 receptor on the cell surface is dimerized. Dimerization of MUC1* on the surface of stem cells not only increases their rate of growth but also staves off differentiation. In contrast, the addition of an agent that prevents MUC1 dimerization blocked stem cell growth and in some cases induced cell death within a very short period of time.

As a demonstration, we used bivalent anti-PSMGFR antibody to dimerize MUC1* on the surface of embryonic stem cells, to induce cell growth and used the monovalent FAb of anti-PSMGFR to bind to MUC1* and block receptor dimerization. The details are that H9 low passage number hESCs were grown over matrigel for 7 days. To one set of wells was added bivalent anti-PSMGFR; to a second set was added the monovalent anti-PSMGFR; and to a third set buffer was added as a control. The results were that the addition of monovalent anti-PSMGFR caused stem cells to die within 8 hours. Cells immediately rounded up and within hours floated to the top of the media. A live/dead assay showed that these cells took up ethidium thus confirming that they were in fact dead. In contrast, wells to which bivalent anti-PSMGFR was added, showed healthy stem cell growth that quantitatively was significantly more than in the control wells. Colonies were larger and in general had less differentiated cells.

In another demonstration, we used a bivalent antibody to proliferate stem cells. One can use any agent that causes the dimerization of MUC1* for this purpose. For example, natural or unnatural ligands of the MUC1 receptor may be used to accelerate stem cell growth. For example, NM23 is a natural ligand of MUC1* and is also useful for stimulating stem cell growth. Since NM23 exists in several multimerization states, concentrations at which the ligand is a dimer are especially preferred. The protein 14-3-3 is also a ligand of MUC1* and may similarly be used to stimulate stem cell growth. Especially preferred are dimeric ligands and/or the addition of ligands at a concentration that is characterized by the dimeric state. Peptides derived from natural ligands may be artificially dimerized and added to growing stem cell to accelerate growth and to inhibit differentiation. Similarly, methods such as phage display can be used to identify de novo peptides that bind to the PSMGFR sequence (MUC1* extracellular domain). These peptides may be dimerized either by single chain synthesis, chemical coupling, or via disulfide bonding to generate new MUC1* ligands and growth factors. The monomeric peptide may be added to growing stem cells to initiate differentiation. It is not intended for these methods to be limited to use on stem cells. The inventors have previously described small molecule inhibitors of MUC1* designed as cancer therapies. These small molecules may be synthesized as dimers and used to stimulate stem cell growth.

The invention anticipates using the above methods for the manipulation of cells whose growth is mediated by the MUC1* receptor, including but not limited to progenitor and precursor cells, neutrophils and the like and cancer cells.

Cell Expansion Through MUC1 Receptor Manipulation

The receptor may be purposely activated to promote the growth of these stem cells or pre-cursor cells in vitro, ex vivo, and/or in vivo for therapeutic, research and other purposes.

The MUC1 receptor may be purposely activated by: 1) inducing receptor cleavage; 2) treating cells bearing the receptor with an activating ligand which may be an agent that dimerizes the receptor, including an antibody that binds to a portion of the receptor that is accessible; 3) transfecting cells with the MUC1 receptor or the MGFR portion thereof, and/or delivering a gene or other mechanism that allows a cell to express the MUC1 receptor and/or its activating ligands.

Bivalent antibodies directed against the PSMGFR or nat-PSMGFR sequence of the MUC1 receptor have been shown to stimulate the growth of MUC1 presenting tumor cells (FIGS. 2-6). Similar antibodies can be used to activate the MUC1 receptor and promote the proliferation of a variety of non-cancerous cells including immature cells or stem cells. Anti-PSMGFR or anti-nat-PSMGFR are examples of such antibodies. However any antibody directed against any region of the MGFR may be used to stimulate the growth of MUC1-positive cells wherein the nat-PSMGFR portion of the receptor is accessible. Natural ligands of the MUC1 receptor or functional mimics thereof may also be used to promote MUC1-mediated cell growth. Ligands of the MUC1 receptor may include but are not limited to NM23, 14-3-3, and/or cathepsin D.

Alternatively, enzymes such as TACE/ADAM17 or MT1-MMP/MMP14 can be administered to cells presenting the full length receptor to enhance cleavage to the growth factor receptor form and thus promote cell growth (FIG. 21). Any enzyme that is able to cleave the MUC1 receptor such that the PSMGFR portion of the receptor becomes exposed would constitute an acceptable method for promoting the proliferation of MUC1-presenting cells.

Methods of the invention including antibodies that dimerize the MGFR portion of the MUC1 receptor may be administered to neutrophils or their precursors in vitro or ex vivo, then depleted of the antibody and re-introduced to the patient. Similarly, agents that increase the cleavage of MUC1 to the growth factor receptor form can be used to stimulate the growth of immature cells, such as but not limited to stem cells, progenitor, precursor cells, neutrophils, and neutrophil pre-cursors. Ligands, such as growth factors, that activate the MGFR portion of the MUC1 receptor may also be used to stimulate the growth of these cell types. These methods may be used to stimulate the proliferation of stem cells, neutrophils, or any other cell that presents the cell surface receptor MUC1, where cell proliferation would be desired.

A form of gene therapy designed to stimulate the growth of immature cells such as stem cells, progenitor cells, neutrophils, or neutrophil pre-cursors, comprises introducing DNA that codes for MUC1 or preferably the truncated MUC1, consisting essentially of the PSMGFR, into immature cells such as stem cells, progenitor cells, neutrophils or like cells wherein proliferation is desirable. DNA that encodes the MUC1 ligand or antibodies that bind to the portion of the receptor that remains attached to the cell surface after cleavage may be introduced to stimulate the growth of the new cells. DNA encoding the G-CSF receptor may be introduced in parallel since G-CSF stimulates the expression or cleavage of the MUC1 receptor.

In another embodiment, the invention involves administering or adding G-CSF in combination with an agent that activates MUC1 and/or an agent that dimerizes the MUC1 receptor and/or assists in cleaving MUC1 to cause the proliferation of stem cells, neutrophils, and other cell types that present both the MUC1 receptor and/or the G-CSF receptor.

Consistent with these findings, the amount of MGFR that is accessible on cells (tissues) can be correlated with tumor aggressiveness and aggressive cell growth. Therefore, antibodies that recognize the MGFR portion of the receptor and have been shown to trigger MUC1-mediated cell growth can be used to promote cell growth in non-cancerous cells that express MUC1 wherein the PSMGFR portion of the receptor is accessible. Examples of such cells include but are not limited to stem cells, neutrophils, mast progenitor cells, and other immature cells.

A recent publication showed that when breast cancer patients were treated with G-CSF (granulocyte colony stimulating factor) their serum levels of shed MUC1 greatly increased (G-CSF induces elevation of circulating CA 15-3 in breast carcinoma patients treated in an adjuvant setting. Briasoulis E, Andreopolou E, Tolis C F, Bairaktari E, Katsaraki A, Dimopoulos M A, Fountzilas G, Seferiadis C ans Pavlidis N. (2001) Cancer, 91, 909-917). Their investigation showed that the increase in levels of shed MUC1 correlated with an increase in the number of neutrophils. These researchers reported that these neutrophils bear an increased number of MUC1 receptors in the cytoplasm, but not on the surface of the neutrophil. Applicant showed that the conclusions reached in this publication, i.e. that the neutrophils did not express MUC1 on their surface, is an erroneous conclusion because the studies cited used an antibody (CA 15.3) that recognizes the tandem repeat portion of the MUC1 receptor, (see FIGS. 8, 10 and 14). FIGS. 8 and 10 show that both breast and lung cancer cells stain positive for the MUC1 receptor in the cytoplasm, but not on the cell surface when probed with VU4H5, which is an antibody that binds to the tandem repeat portion of the receptor. However, when the adjacent section is probed with anti-PSMGFR, which binds to the PSMGFR portion of the receptor, it can be seen that a cleavage product of the receptor completely coats the cell surface. The Applicant previously showed that a cleavage product of the MUC1 receptor that is essentially comprised of the PSMGFR sequence, functions as a growth factor receptor.

These data are consistent with the idea that G-CSF stimulated the proliferation of neutrophils and that the enhanced proliferation is due to an increased number of MUC1 receptors that are present on the surface of neutrophils in the cleaved form, which has been stripped of the tandem repeat section, and that this proteolyzed form of MUC1 is the growth factor receptor that is driving proliferation. Further, dimerization of the MGFR portion of MUC1 triggers this cell proliferation, optionally with an agent for doing so. Therefore, agents that dimerize MUC1 can be used to stimulate the growth of certain cell types, such as in vitro, ex vivo, in vivo, or in situ. Specifically, stem cells, progenitor, precursor cells, neutrophils and the like, can be stimulated to proliferate by adding agents that dimerize or multimerize the MGFR portion of MUC1.

As reported in the literature, G-CSF enhances the production of MUC1 and specifically of the cleaved form of MUC1 that acts as a growth factor receptor. Therefore, strategies to stimulate the growth of stem cells, neutrophils and other cell types that present both the MUC1 receptor and/or the G-CSF receptor may include agents that act on both the G-CSF receptor and the MUC1 receptor, specifically the portion that remains attached to the cell surface after receptor cleavage. That is to say that stem cell proliferation as well as increased neutrophil populations may be achieved by co-stimulation of both receptors, either simultaneously or in staggered treatment protocols.

Non-Tumor Cell Proliferation

In yet other embodiments, the invention provides methods for treating a subject for which stem cell or any progenitor cell would have therapeutic value, or other condition requiring treatment with one or more of the antibodies or antigen-binding fragments thereof of the invention. The method involves administering to the subject an antibody or antigen-binding fragment thereof in an amount effective to expand the stem cell or progenitor cell in the subject. In certain embodiments, any of the above-mentioned antibodies or antigen-binding fragments thereof, especially those which specifically bind to MGFR, PSMGFR, nat-PSMGFR and so on can be used. In certain preferred embodiments, the antibody or antigen-binding fragment thereof is administered in an amount effective to enhance the interaction of the MUC1 receptor for example, MGFR, that remains attached to a cell after shedding of an interchain binding region of the MUC1 receptor. In an embodiment of the method, particularly in which the antibody or antigen-binding fragment thereof specifically binds to MGFR, such a treatment method can involve administering to the subject the antibody or antigen-binding fragment thereof in an amount effective to cause inductive dimerization of a growth factor receptor, such as cleaved MUC1.

There are many uses for techniques to stimulate the growth of stem cells, progenitor cells, neutrophils, mast progenitor cells and their precursors. A single stem cell can proliferate and differentiate to become an entire organ. Methods to manipulate the growth and/or differentiation of stem cells and progenitors would find uses in tissue regeneration, organ generation, expansion of depleted cell populations to treat conditions such as spinal column injury and Alzheimer's disease. Growth of these cells may be carried out in vitro or ex vivo. For example, a patient's own cells could be expanded then re-introduced to the patient. Alternatively, stimulating agents may be introduced in vivo, either alone or in combination with stem cells or stem cell-like cells, e.g. at a site of tissue or nerve injury. In other embodiments, allogeneic cells may be used in the case of stem cells.

In a preferred embodiment, agents that are directed to the MGFR portion of the MUC1 receptor, such as a dimerizing antibody, can be used to enhance white blood cell count in patients receiving therapies that induce neutropenia. These agents may be directly administered to patients being treated for non-cancerous conditions or MUC1-negative cancers, as well as other immuno-compromised patients. Alternatively, a patient's own neutrophils or precursors thereof may be removed from the patient and expanded, using methods of the invention, then re-introduced into the patient. For example, this would eliminate the need for bone marrow transplants for patients who have undergone extensive radiation or other methods that destroy the bone marrow. Conditions such as leukemias may also be treated with this method to restore the patient's immune system and blood profile.

In a preferred embodiment, the MUC1* receptor manipulated for therapeutic applications involve the selection and amplification of stem cells to restore nerve function for diseases and injury, to replace organ tissue for cases where organ tissue is lost because of atrophy, disease, surgery, and injury. Therapeutic applications of methods of the invention also include amplifying and directing differentiation of ectoderm stem cells for generating skin for wound repair, scar repair, burn, and cosmetic purposes. Stem cells from bone marrow extracts can be amplified for therapeutic purposes.

The invention further anticipates using methods for the identification, isolation and manipulation of stem cells for cosmetic uses. For example, for identifying, harvesting and amplifying hair folicules for hair transplant, for head hair, eye lash, eye brow and the like. Stem cells that differentiate into fat cells or breast tissue are harvested and transplanted for breast augmentation, lip, cheek, and body re-shaping or enhancement. Applications to the regeneration and restoration of teeth, enamel, dentin and the like are also included within the purview of the invention. Endocrine cell replacement is a part of the invention for example for cells including but not limited to beta cells of the islets of langerhorn for the treatment of diabetes.

Methods of the invention are also suitable for the treatment of conditions that include but are not limited to bone injury, osteoporosis, neurodegenerative diseases, arthritis, multiple sclerosis, degenerative maladies caused by autoimmune diseases, stroke, traumatic brain injury, spinal cord injury whether due to traumatic injury or degenerative diseases such as multiple sclerosis, arterial and venous conditions, aneurysm, macular degeneration, blindness, paralysis, for the treatment of degenerative disease, including muscular degenerative diseases, MS, AD, PD, CJD, Lou Geurrig's disease, stroke, Plaget's syndrome, cerebro palsy, sickle cell anemia, burn victims, new skin, new tissue, limb regeneration, organ regeneration, nerve and brain cell regeneration. Methods of the invention may also be used to regenerate the hair-like cells, cochlear cells, to enable or restore hearing.

Similar to a blood bank, supplies of toti-pluri, and multi-potent stem cells and other pre cursor cells may be harvested and sorted into categories of differentiation for subsequent amplification and introduction or re-introduction into a recipient.

Immature Cell Expansion

Immature cells include somatic stem cells, embryonic stem cells, cord blood stem cells, and other not fully differentiated cells. Adult stem cells also known as somatic stem cells, are undifferentiated cells found among differentiated cells of a specific tissue and are mostly multipotent cells. They are already being used in treatments for over one hundred diseases and conditions. Certain adult stem cells termed “spore-like cells” are present in all tissues (Vacanti, M. P., A. Roy, J. Cortiella, L. Bonassar, and C. A. Vacanti. 2001, J Cell Biochem 80:455-60.).

Embryonic stem cells are cultured cells obtained from the undifferentiated inner mass cells of an early stage human embryo are totipotent. Cord blood stem cells are derived from the blood of the placenta and umbilical cord after birth. Cord blood stem cells are used to treat without limitation Gunther's disease, Hunter syndrome, Hurler syndrome, Acute lymphocytic leukemia.

Allogeneic Treatment is Contemplated in the Present Invention.

Moreover, in particular, bone marrow contains two types of stem cells: hematopoietic (which can produce blood cells) and stromal (which can produce fat, cartilage and bone). Stromal stem cells have the capability to differentiate into many kinds of tissues, such as nervous tissue. Hematopoietic stem cells give rise to the three classes of blood cell that are found in the circulation: leukocytes, red blood cells (erythrocytes), and platelets (thrombocytes). Pluripotential hemopoietic stem cells or pluripotential hematopoietic stem cells (PHSCs) are stem cells found in the bone marrow. PHSC are the precursor cells which give rise to all the blood cell types of both the myeloid and lymphoid lineages. This includes monocytes and macrophages, neutrophils, basophils, eosinophils, T-cells, B-cells, NK-cells, microglia, erythrocytes (red blood cells), megakaryocytes (e.g. platelets), and dendritic cells.

As discussed herein, a proteolyzed form of the MUC1 receptor functions as a primal growth factor receptor to drive the proliferation of a number of cell types, including but not limited to immature cell types such as stem cells and progenitor cells. Table 3 lists the cell types that are known to express MUC1 and treatments for which methods of the invention would be suitable. Additionally, cell types that do not express MUC1 could be stimulated to proliferate by genetically manipulating the cells to express MUC1 or a MUC1 truncation mutant and then applying methods of the invention to stimulate the MUC1 receptor and induce or enhance cell proliferation.

NM23

Activation of the MUC1* growth factor receptor is an autocrine process. Cells that express the growth factor receptor form of the MUC1 receptor, MUC1*, also produce the ligand that activates the receptor. The antibody stimulation experiments demonstrated that dimerization of the MUC1* receptor stimulates cell growth by triggering the MAP kinase cell proliferation cascade. To determine whether or not MUC1 cancer cells also produced the dimerizing ligand, a nanoparticle experiment was devised that detects the presence of ligands that dimerize the MUC1* peptide. Histidine-tagged PSMGFR (MUC1*) peptide was immobilized on NTA-nickel SAM coated gold nanoparticles. Extracts and supernatants from a panel of MUC1-positive breast tumor cell lines were added to the MUC1*-bearing nanoparticles. Solutions that turned from pink to blue contained ligands that dimerized the particle-immobilized MUC1* peptides. (Due to an inherent optical property of gold nanoparticles, they appear pink when in a homogeneous suspension but turn blue when drawn close together, for example, when particle-immobilized ligands bind to a common target.) FIG. 22 displays the results of the nanoparticle experiment. Cancer cell extracts from breast cancer cell lines, CRL-1504, T47D, CRL-1500 and CRL-1902 were separately added to MUC1* peptide-presenting nanoparticles in Column A. In Column B, the same cancer cell extracts were added to nanoparticles that presented an irrelevant peptide; these wells did not turn blue because the ligand in the cell extract was not recognized by the irrelevant peptide. The wells in Column A turned blue at a rate that roughly reflected the amount of MUC1 that each cell line produced. For example, CRL-1902 cells present low levels of the MUC1 receptor. The nanoparticle well to which CRL-1902 extracts were added required many more hours to turn blue than did the T47D well. T47D cells present high levels of MUC1 receptor.

NM23 is the ligand that dimerizes and activates the MUC1* growth factor receptor. T47D cancer cell extracts were immunoprecipitated with magnetic beads that were pre-bound with histidine-tagged PSMGFR (MUC1*) peptide. The captured MUC1* binding species were then separated by SDS-PAGE. A western blot was then performed by probing the gel with an antibody that recognized NM23 H1. FIG. 23 is a western blot that shows that the species that was immunoprecipitated, from T47D supernatants, with anti-MUC1*, reacts with anti-NM23 and is the expected molecular weight of NM23. Thus, the ligand to MUC1 is NM23. Note that Lane 2, into which was loaded a sample from 3Y1 supernatants, shows no detectable levels of NM23.

Expression of the MUC1 receptor stimulates the expression of NM23 (FIG. 24). FIG. 24 shows the results in an extension of the previous experiment. Here, cell supernatants (upper gel) or lysates (lower gel) were immuno-precipitated with MUC1* attached to magnetic beads. Captured MUC1*-binding species were then released and run out on a gel, then blotted with an antibody that recognizes NM23. The IP/western blot of FIG. 24 shows that MUC1-negative cell line 3Y1 does not produce NM23 in the lysate or in the supernatant (Lane 1). However, after the transfection of full-length MUC1 (Lane 2) (which the experiments show is efficiently cleaved to yield MUC1*) or MUC1* (Lane 3), NM23 is then detected in the lysates. Lane 4 shows that HEK 293 cells that have been transfected with MUC1*, have detectable levels of NM23 in their lysates and their supernatants. Lanes 5, 6 and 9 are breast cancer cell lines and show detectable levels of NM23 in the lysate and supernatants. Lane 8 is BT474 which is a MUC1-positive breast cancer cell line that produces relatively low levels of MUC1 receptor on their surface. Into Lane 7 was loaded HeLa cell extracts, which express low levels of MUC1 receptor. Taken together, these results, especially the fact that MUC1-negative cells that do not express NM23, begin to express NM23 after MUC1 transfection, strongly argue that the stimulation of the MUC1 receptor triggers expression of its ligand NM23.

NM23 stimulation of MUC1* is self-regulated by an autocrine feedback loop. NM23 can exist as a monomer, dimer, tetramer or hexamer (reviewed in Lascu, et al. J. Bioenerg. Biomemb 2000 32(3):227-36). The NM23 dimer activates cell growth by dimerizing the MUC1* receptors. Higher order multimers of NM23, such as the hexamers, inhibit MUC1-dependent cell growth, by inducing clustering of the MUC1 receptors. Cancer or unchecked cell growth can be caused by a defect in this self-regulation of the MUC1 receptor. Mutations in NM23 that inhibit the formation of the protective hexamers can result in cancers. These mutations that inhibit hexamer formation show increased levels of NM23 dimers, which activate MUC1-dependent cell growth by dimerizing the MUC1* receptor. The mutant in NM23-H1, S120G is common in neuroblastoma, and it has been shown that the S120G mutation results in the reduction of hexamers and an increase in dimers in solution when compared to wild type protein (Kim, et al. Biochem Biophys Res Comm 2003 307: 281-9). Transfection of wild type NM23-H1, or mutants that did not affect hexamer formation inhibited migration of prostate cancer cells (Kim, et al. Biochem Biophys Res Comm 2003 307: 281-9) and breast cancer cells (MacDonald, et al. J Biol Chem 1996 271(41): 25107-16) in in vitro invasiveness assays, while transfection of mutant NM23-H1 constructs that harbored the S120G mutation did not inhibit invasiveness, and in some experiments, enhanced invasiveness (MacDonald, et al. J Biol Chem 1996 271(41): 25107-16).

NM23 treatments: The growth of stem cells and progenitor cells is stimulated by providing the cells with NM23. In a preferred embodiment, NM23 is provided at concentrations that favor ligand dimerization, such as S120G. In another embodiment, mutant forms of NM23 that preferentially form dimers are provided for stimulating stem cell and progenitor cell growth and for delaying differentiation.

NM23 is an activating ligand of the MUC1 receptor. It triggers cell growth by dimerizing the MUC1* domain of the MUC1 receptor. Treating stem cells and progenitor cells with a dimeric form of NM23 stimulates cell growth and staves off differentiation. The other growth factor receptor that exists on stem cells is the FGF receptor. Stimulation of the FGF receptor with bFGF has been shown to stave off stem cell differentiation. Withholding bFGF from stem cells for 14-21 days causes the cells to differentiate. By analogy, stimulating the MUC1* growth factor receptor that is on stem cells and some progenitor cells, particularly hematopoietic stem cells and progenitors with the dimeric form of NM23 will delay differentiation.

A Review of the Current State of Knowledge in this Area Shows the Level of Confusion that Exists Regarding the Role of NM23 in Differentiation.

Okabe-Kado, et al (Cancer Research 1985 45: 4848-52) first identified an inhibitory factor of differentiation (as defined by the induction of phagocytic activity post-treatment of cells with dexamethasone) of murine myeloid leukemia M1 cells contained within the cultured medium of those cells. In a follow-on paper (Okabe-Kado, et al, BBRC 1992 182(3):987-94), the inhibitory factor was identified as murine NM23-H2. Further work demonstrated the inhibition of erythroid differentiation of the erythroid leukemia cell lines K562, HEL and KU812F cells (Okabe-Kado, et al. Biochimica Biophysica Acta 1995 1267:101-106). In these experiments, erythrocytic differentiation was defined as positive staining for benzidine, and differentiation was induced by TGF-β1, Hemin and Retinoic Acid.

These experiments also demonstrate that recombinant human NM23-H1 and -H2, in the form of GST fusions block erythroid differentiation of HEL cells by TGF-β11, and that kinase-deficient human NM23-H2, and truncated NM23-H2 (aa1-60) are also capable of inhibiting differentiation of HEL cells by TGF-β1. Similar work done by this group around the same time (Okabe-Kado, et al FEBS Letters 1995 363: 311-315), showed that recombinant GST fusions of mouse, human, and rat NM23 1 and 2 proteins inhibited inhibition of differentiation of murine myeloid leukemia M1 cells, as defined by the induction of phagocytic activity post-treatment of cells with dexamethasone, and that the kinase activity of NM23-H2 is not required for this activity, and that N-terminal regions of NM23-H2 (aa1-60 and aa1-108) contain wild-type inhibitory activities.

So far, these papers discuss the inhibition of differentiation that they claim may be due to the action of NM23. No target receptor for NM23 is identified. NM23 is discussed in general without any importance given to the multimerization state of NM23. NM23 can exist as a monomer, dimer, tetramer or hexamer (reviewed in Lascu, et al. J. Bioenerg. Biomemb 2000 32(3):227-36). The present application discloses that as a dimer, NM23 dimerizes the MUC1* receptor and triggers cell growth. At higher multimerization states, e.g. hexamers, they no longer dimerize the receptor to trigger cell growth or stave off differentiation. To the contrary, they inhibit both these activities by re-clustering the MUC1 receptors and inhibiting its growth factor receptor activity. The second point to note about this research is that the authors were studying the effects of NM23 in leukemia and or cancer cell lines, wherein NM23 could likely be a mutant form that prefers dimer formation to hexamer formation (Kim, et al. Biochem Biophys Res Comm 2003 307: 281-9). Further, the recombinant NM23 that is cited as having these same inhibitory effects on differentiation was expressed as a GST fusion protein; GST is a dimer and makes GST fusion proteins dimers.

More recent work done by Willems, et al. (Experimental Hematology 2002 30:640-8) showed that the addition of recombinant NM23-H1, -H2, or -H3 did not induce growth of hematopoietic stem cells (CD34++CD38−) or expand their ability to form colonies, though NM23-H3 may have induced colony forming efficiency to a slight degree. However, treatment of hematopoietic progenitor cells (CD34+CD38+) with these proteins reduced the differentiation of these cells into granulocyte, macrophage, or granulocyte-macrophage colonies (CFU-G, CFU-M, or CFU-GM), and expanded the formation of erythroid colonies, erythroid bursts, or mixed colonies (CFU-E, BFU, or CFU-GEMM). Colony morphology was scored by microscopy.

This last body of work uses recombinant NM23 that may be in any one of four multimerization states, wherein the monomer, dimer (activates growth and delays differentiation) and hexamer (inhibits growth and induces differentiation) produce vastly different results.

Similarly, a suitable treatment for patients suffering from MUC1-positive cancers consists of providing NM23 that is able to form hexamers, either through direct administration or via a gene therapy approach.

MUC1* Activation

Based on the findings that MUC1* mediates growth in cancer cells, experiments were devised to show whether or not stem cell growth could be manipulated by altering the dimerization state of MUC1*. A set number of human embryonic stem cells, H9p55, were plated onto matrigel. Cells were treated with either: 1) the bivalent anti-MUC1* antibody (rabbit polyclonal antibody raised the PSMGFR sequence), which dimerizes the MUC1* portion of the receptor; 2) the monovalent form of this same antibody, which binds to a single MUC1* receptor and prevents dimerization; or 3) nothing added as a control. The most striking result was that nearly all the cells, to which the monovalent anti-MUC1* antibody was added, died within 8 hours of the treatment, see FIGS. 25 and 26. It was noted that when free MUC1* peptide was added, see FIG. 27, there are more live cells, presumably because the free MUC1* peptide competes with the cell surface attached MUC1* for binding to the inhibitory monovalent antibody. A complete reversal of the monovalent antibody induced cell death was not expected since the free peptide could also compete with the cell surface MUC1* receptor for binding to the activating ligand. Relying only on visual inspection, it was not possible to distinguish differences between the control cells and cells to which bivalent anti-MUC1* was added, compare FIGS. 28 and 29 and FIGS. 30 and 31. Therefore, the experiment was repeated but the analysis was done in a manner that resultant cell numbers could be quantitated.

A given number of cells H9p55 were added to each well. Again, the cells were treated with either bivalent anti-MUC1*, monovalent anti-MUC1* or no antibody as a control and then counted on a cell counting instrument. The graph of FIG. 32 summarizes the results. Again, treatment with the monovalent anti-MUC1* antibody caused cell death within 8 hours. The addition of the bivalent anti-MUC1* increased cell growth, albeit modestly, compared to the control cells. The graph of FIG. 33 gives the details of this experiment. Drawing attention to the first and third clusters of bars that correspond to bivalent antibody added and no antibody added respectively, it is noted that the addition of the bivalent anti-MUC1* increases cell count. Comparing the first bar in each group: B-bFGF-NO peptide (bivalent anti-MUC1* added-bFGF added-no MUC1* peptide added) compared to bFGF-NO peptide (no bivalent antibody added-bFGFadded-no MUC1* peptide added), it is noted that the addition of the bivalent anti-MUC1* increases cell growth. Comparing the second bar from the first and third grouping, it is noted that the addition of the MUC1* peptide to the same two previous conditions causes a drop in the cell count, presumably because the peptide competes with the cell surface attached MUC1* for the stimulatory binding to the activating ligand for both conditions and competes for binding to the antibody also in the first condition.

Therefore, bivalent anti-MUC1* can be added to stem cells to enhance their growth. Additionally, the stem cells can be grown in the absence of fibroblast feeder cells that provide unknown growth factors to the cells. These uncharacterized growth factors presumably also include factors that trigger the undifferentiated stem cells to begin differentiation. Growing the cells in the presence of bivalent anti-MUC1* will allow the cells to be grown with minimal addition of supplemental factors which will hold off differentiation. These methods are not intended to be limited to the enhancement of stem cell growth. Many progenitor cells express MUC1* on their surface and their proliferation can be amplified using antibodies and agents that dimerize the MUC1* portion of the MUC1 receptor.

For example, it has been disclosed in the present invention that NM23 as a dimer activates the MUC1* receptor and drives the growth of cancer cells and stem cells. Therefore, NM23 can be added to stem cells and progenitor cells to stimulate cell growth and to stave off differentiation of these cells for research and therapeutic purposes. Mutants of NM23 that prevent or inhibit the formation of higher order multimers (Kim, et al. Biochem Biophys Res Comm 2003 307: 281-9), such as hexamers, can be added to stimulate stem cell growth without the formation of the hexamers which re-cluster the MUC1 receptor and inhibit cell growth.

Antibodies

Peptides used for antibody production may or may not be glycosylated prior to immunizing animals. The sequence of these peptides need not exactly reflect the sequence of MUC1 receptor as it exists in the general population. For example, the inventor observed that antibodies raised against the PSMGFR peptide variant var-PSMGFR (SEQ ID NO:12), having an “-SPY-” motif have a higher affinity and greater specificity for the MUC1 protein than antibodies raised against the actual native sequence (i.e. nat-PSMGFR, SEQ ID NO: 10), having an “-SRY-” motif. One may also, in certain embodiments, introduce mutations into the PSMGFR peptide sequence to produce a more rigid peptide that may enhance antibody production. For example the R to P mutation in the var-PFMGFR sequence of SEQ ID NO:12 may actually have provided a more rigid peptide and was thus more immunogenic. Another method for producing antibodies against regions of peptides that are not particularly immunogenic, such as the IBR or TPSIBR is to tag the specific peptide sequence with an irrelevant sequence in which the amino acids are of the D-form and thus act to stimulate the immune response of the host animal. Peptide sequences that are used to immunize animals for antibody production may also be glycosylated. The MUC1 peptide sequences that were used herein for drug screening and to generate cognate antibodies were derived from the human species of MUC1. Since there is considerable conservation across species for the PSMGFR and IBR and some portions of the UR, it is anticipated that MUC1 peptides whose sequences are derived from other species can also be used in drug screens and to generate antibodies for these same purposes.

In certain aspects, the invention provides antibodies or antigen-binding fragments thereof. In one embodiment, the invention provides an antibody or antigen-binding fragment that specifically binds to MGFR. In certain embodiments, the above-mentioned antibodies or antigen-binding fragments thereof specifically bind to PSMGFR. In certain such embodiments, the antibodies or antigen-binding fragments thereof can specifically bind to the amino acid sequence set forth in SEQ ID NO:10 or a functional variant or fragment thereof comprising up to 15 amino acid additions or deletions at its N-terminus or comprising up to 20 amino acid substitutions; in other embodiments, it specifically binds to the amino acids set forth in SEQ ID NO:10 or a functional variant or fragment thereof comprising up to 10 amino acid substitutions; in other embodiments, the antibodies or antigen-binding fragments thereof specifically bind to the amino acid set forth in SEQ ID NO:10 or a functional variant or fragment thereof comprising up to 5 amino acid substitutions; and in yet another embodiments the antibodies or antigen-binding fragments thereof specifically bind to the amino acid sequence set forth in SEQ ID NO:10. In certain embodiments, the antibody or antigen-binding fragment of the invention is a human, humanized, xenogenic or a chimeric human-non-human antibody or antigen-binding fragment thereof. In certain embodiments, the antibodies or antigen-binding fragments thereof of the invention comprise an intact antibody or an intact single-chain antibody. For antibodies or antigen-binding fragments that are monovalent, in certain embodiments, they may comprise a single-chain Fv fragment, a Fab′ fragment, a Fab fragment, or a Fd fragment. For antibodies or antigen-binding fragments of the invention that are bivalent, certain embodiments comprise an antigen-binding fragment that is a F(ab′)₂. In certain such compositions, the antibody or antigen-binding fragment thereof can be polyclonal, while in other embodiments it can be monoclonal.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

As is now well known in the art, the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205, which are incorporated by reference herein in their entirety. Such antibodies, or fragments thereof are within the scope of the present invention.

In certain embodiments, fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

In certain embodiments the present invention comprises methods for producing the inventive antibodies, or antigen-binding fragments thereof, that include any one of the step(s) of producing a chimeric antibody, humanized antibody, single-chain antibody, Fab-fragment, F(ab′)₂ fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor, 1988. The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogeneic antibodies. The general principle for the production of xenogeneic antibodies such as human antibodies in mice is described in, e.g., WO 91/10741, WO 94/02602, WO 96/34096 and WO 96/33735. As discussed below, the antibodies, of the invention may exist in a variety of forms (besides intact antibodies; including, for example, antigen binding fragments thereof, such as Fv, Fab and F(ab′)2, as well as in single chains (i.e. as single chain antibodies); see e.g., WO88/09344.

Thus, as will be apparent to one of ordinary skill in the art, the present invention also provides, in certain embodiments, for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Chemical Derivatives of Antibodies and Formulations

In certain embodiments, the present invention relates to compositions comprising the aforementioned antibodies or antigen-binding fragments of the invention or chemical derivatives thereof. The composition of the present invention may further comprise a pharmaceutically acceptable carrier. The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes, e.g., for intranasal administration. Formulations for rectal or vaginal administration may be presented as a suppository with a suitable carrier.

A therapeutically effective dose refers to that amount of antibodies and/or antigen-binding fragments of the invention ameliorate the symptoms or conditions of the disease being treated. Therapeutic efficacy and toxicity of such compositions can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The biological activity of the antibodies and/or antigen binding fragments thereof, of the invention indicates that they may have sufficient affinity to make them candidates for drug localization to cells expressing the appropriate surface structures, e.g. MGFR. Thus, targeting and binding to cells of the antibodies and/or antigen binding fragments thereof, of the invention could be useful for the delivery of therapeutically or diagnostically active agents (including targeting drugs, nucleic acids and nucleic acid analogs, DNA sequences, RNA sequences, lipids, proteins and gene therapy/gene delivery. Thus, the antibody and/or antigen binding fragments thereof, of the invention can be labeled (e.g., fluorescent, radioactive, enzyme, nuclear magnetic, colloid, other signaling entity, etc.) and used to detect specific targets in vivo or in vitro including “immunochemistry” like assays in vitro. In vivo they could be used in a manner similar to nuclear medicine imaging techniques to detect tissues, cells, or other material expressing MGFR. Another method of the invention involves using antibodies that bind to the MGFR portion of the MUC1 receptor as a method for sorting and/or isolating cells that need to be expanded. Once sorted, these cells would be expanded in vitro. New genetic material, for example that codes for co-receptors and/or activating ligands, may be added to these selected cells either before or after expansion. Activating antibodies may be depleted from the cell population before introduction to the subject. Yet another method involves delivering a therapeutically active agent to a patient. The method includes administering at least one antibody or an antigen-binding fragment thereof and the therapeutically active agent to a patient. Preferably, the therapeutically active agent is selected from drugs, nucleic acids and nucleic acid analogs, sequences, RNA sequences, proteins, lipids, and combinations thereof.

MUC1 Modulated Cell Growth

The invention encompasses methods and agents to modulate cell growth that is mediated by MUC1.

Agents for the modulation of proliferation mediated by MUC1 are directed to the region of MUC1 that we refer to herein as MUC1* or MGFR. Such therapeutic agents are, more broadly, directed toward portions of MUC1 that remain attached to the cell surface and are not released and shed from the cell surface. In a preferred embodiment, therapeutic agents are directed toward the region of MUC1 that consists primarily of the PSMGFR sequence. They may be directed toward portions or fragments of the PSMGFR sequence. Additionally, the PSMGFR sequence may be N-terminally extended and this extended PSMGFR sequence may be the target therapeutic region. In another embodiment, the target region of MUC1 may be a frame shift of the PSMGFR such that the target sequence is extended 6-12 amino acids on the N-terminal side and truncated by a similar number of amino acids on the C-terminal side. Therapeutic agents that target this region of MUC1 include antibodies, antibody fragments, monovalent antibodies, bispecific or dual domain antibodies, small molecules, proteins, peptides and the like. Antibodies and antibody fragments may be optionally pegylated, conjugated to other substances, proteins such as albumin, peptides, and/or polymers as long as the conjugated or hybrid antibody species retains an ability to bind to a portion of the MUC1 protein defined as MGFR or that contains at least 6 to 9 contiguous amino acids of the PSMGFR or PSMGFR-nat or PSMGFR-var sequence.

Agents for the modulation of growth mediated by MUC1 may be directed to extracellular or intracellular portions of MUC1. Antagonists of interactions between the MUC1 and its ligands are inhibitors of MUC1 mediated cell growth and can be used as therapeutic agents to inhibit the growth of or kill cancer cells and cancer stem cells. Agonists or antagonists of interactions between MUC1 cytoplasmic tail and its ligands can be used to enhance or inhibit, respectively, cell growth. Agents that inhibit phosphorylation of ERK2 for example would inhibit growth mediated by MUC1. Agents that inhibit interactions, either extracellular or intracellular, between MUC1 and other growth factor receptors including Her2 or EGF receptor or VEGF receptor are anticipated to be growth inhibitors and as such would be especially useful for inhibiting the growth of cancer cells and cancer stem cells. In a preferred embodiment, an antibody raised against at least 6 contiguous amino acids of the PSMGFR peptide sequence is an agent for killing or inhibiting the growth of cancer cells and cancer stem cells. In an especially preferred embodiment, the antibody is monovalent and can bind to at least 6 contiguous amino acids of the PSMGFR peptide. Antibodies and antibody fragments for the inhibition of cancer cell and cancer stem cell growth may be conjugated to toxins or cytotoxic agents and drugs. Antibodies or antibody fragments that bind to the MGFR portion of MUC1 may be optionally designed to induce an immune response in the host for the killing or inhibition of cancer and cancer stem cell growth. Antibodies and fragments thereof can be used as drug carriers and drug delivery agents. The term drug as used herein can mean any therapeutic agent including nucleic acid therapeutics which include RNAi, siRNA and shRNA. Other agents for the inhibition of cancer cell growth and cancer stem cell growth are agents that neutralize NM23. Preferred are agents that neutralize the ability of NM23 to bind to MUC1. Especially preferred are agents that prevent the dimerization of NM23. Such agents include antibodies, antibody fragments, small molecules, proteins, peptides, nucleic acids and nucleic acid analogs, including DNA, RNA, RNAi, siRNA and shRNA.

Portions of MUC1 and/or ligands of MUC1 either alone or conjugated to a carrier or other pharmaceutical reagent may be used to induce a host to produce an immune response against cells bearing MUC1, particularly cancer cells and cancer stem cells. In this way, vaccines can be produced by attaching portions of MUC1 or its ligands to adjuvant, cells, dendridic cells and the like. In a preferred embodiment, peptides derived from the portion of MUC1 that remains attached to the cell surface are used. More preferred are peptides that contain at least 6 contiguous amino acids of the PSMGFR or PSMGFR-nat or PSMGFR-var sequence for stimulating an immune response or vaccine. Similarly, portions or fragments of NM23 may be used either alone, or conjugated to a carrier or adjuvant as immunizing agents to induce an immune response or vaccine. Such vaccines may be used as a treatment or prevention of cancers that include solid tumors and non-solid cancers.

Agonists of MUC1 that act either extracellularly or intracellularly are agents for stimulating stem cell growth and/or delaying differentiation. In one embodiment, antibodies that bind to a portion of MUC1, which is not shed and released from the cell surface, stimulate the growth of stem cells and progenitor cells. In a preferred embodiment, the antibody binds to at least 6 contiguous amino acids of the PSMGFR or PSMGFR-nat or PSMGFR-var sequence.

Proteins

According to another aspect of the invention, a series of isolated proteins or peptides is provided. Inventive peptides may include, but are not limited to, those defined above as PSMGFR and PSMGFRTC, and those listed as SEQ ID NOS: 2-19. Additionally, the invention encompasses any protein, or peptide, not specifically mentioned above that is encoded by any of the isolated nucleic acid molecules of the invention discussed below. The invention also encompasses unique fragments of the above-mentioned proteins or peptides, as well as antibodies made against them, including monoclonal or polyclonal antibodies.

Proteins can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including antigenic peptides (such as are presented by MHC molecules on the surface of a cell for immune recognition) also can be synthesized chemically using well-established methods of peptide synthesis.

The invention also encompasses unique fragments of the inventive proteins or peptides, which in one aspect, are used to generate antibodies. A fragment of any one of the inventive proteins or peptides, for example, generally has the features and characteristics of fragments including unique fragments as discussed herein in connection with nucleic acid molecules. As will be recognized by those skilled in the art, the size of a fragment which is unique will depend upon factors such as whether the fragment constitutes a portion of a conserved protein domain. Thus, some regions of the inventive proteins or peptides will require longer segments to be unique while others will require only short segments, typically between 5 and 12 amino acids (e.g. 5, 6, 7, 8, 9, 10, 11, and 12 amino acids long).

Unique fragments of a protein preferably are those fragments which retain a distinct functional capability of the protein. Functional capabilities which can be retained in a fragment of a protein include interaction with antibodies, interaction with other proteins or fragments thereof, selective binding of nucleic acid molecules, and enzymatic activity. One important activity is the ability to act as a signature for identifying the polypeptide.

Those skilled in the art are well versed in methods for selecting unique amino acid sequences, typically on the basis of the ability of the fragment to selectively distinguish the sequence of interest from non-family members. A comparison of the sequence of the fragment to those on known data bases typically is all that is necessary.

The invention embraces variants of the inventive proteins or peptides described herein. As used herein, a “variant” of a protein is a protein which contains one or more modifications to the primary amino acid sequence of such protein. Modifications which create a protein variant can be made to such protein 1) to produce, increase, reduce, or eliminate—an activity of the protein; 2) to enhance a property of the protein, such as protein stability in an expression system or the stability of protein-protein binding; 3) to provide a novel activity or property to a protein, such as addition of an antigenic epitope or addition of a detectable moiety; and/or 4) to provide equivalent or better binding to a ligand molecule. Modifications to a protein can be made via modifications to the nucleic acid molecule which encodes the protein, and can include deletions, point mutations, truncations, amino acid substitutions and additions of amino acids or non-amino acid moieties. Alternatively, modifications can be made directly to the protein, such as by cleavage, substitution of one or more amino acids during chemical systhesis, addition of a linker molecule, addition of a detectable moiety, such as biotin, addition of a fatty acid, etc. Modifications also embrace fusion proteins comprising all or part of an amino acid sequence of the invention. One of skill in the art will be familiar with methods for predicting the effect on protein conformation of a change in amino acid sequence, and can thus “design” a variant polypeptide according to known methods. One example of such a method is described by Dahiyat and Mayo in Science 278:82-87, 1997, whereby proteins can be designed de novo. The method can be applied to a known protein to vary only a portion of the protein sequence. By applying the computational methods of Dahiyat and Mayo, specific variants of a DOS protein can be proposed and tested to determine whether the variant retains a desired conformation.

The skilled artisan will also realize that certain amino acid substitutions, such as for example conservative amino acid substitutions, may be made in the inventive proteins or peptides to provide “functional variants” of the foregoing proteins or peptides, i.e, variants which possess functional capabilities of the corresponding inventive proteins or peptides. As used herein, a “conservative amino acid substitution” refers to an amino acid substitution which does not alter the relative charge or size characteristics of the protein in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Functional variants having up to 1, 2, 3, 4, 5, 6, 7, 8, 9, 10, 11, 12, 13, 14, 15, 16, 17, 18, 19, 20, or more amino acid substitutions can be made. Similarly, the above or other functional variants can be prepared having, or also having, up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acid additions or deletions at their C- and/or N-terminus. Variants of the proteins or peptides prepared by the foregoing methods can be sequenced, if desired, to determine the amino acid sequence and thus deduce the nucleotide sequence which encodes such variants.

Nucleic Acid

The present invention in another aspect provides nucleic acid sequences encoding a variety of truncated MUC1 receptor proteins, or functional variants or fragments thereof, and other nucleic acid sequences that hybridize to the above nucleic acid sequences under high stringency conditions. The sequence of certain of the nucleic acid molecules of the present invention are presented in Table 2 below as SEQ ID NOS: 21-25, and the predicted amino acid sequences of these genes' protein products, each comprising an isoform of a truncated MUC1 receptor protein, are presented in Table 1. The invention thus involves in one aspect peptide sequences representing truncated isoforms of the MUC1 receptor, genes encoding those peptide sequences and functional modifications and variants of the foregoing, useful fragments of the foregoing, as well as therapeutic products and methods relating thereto. The peptides referred to herein as truncated MUC1 receptor proteins include fragments of the full length MUC1 receptor but do not include the full length MUC1 receptor protein (i.e. SEQ ID NO:1). Likewise, nucleic acid molecules that encode the various truncated isoforms of the MUC1 receptor described herein can include fragments of the MUC1 gene coding region, but do not include the full length MUC1 coding region.

According to one embodiment of the invention, an isolated nucleic acid molecule is provided. The isolated nucleic acid molecule is selected from the group consisting of:

(a) nucleic acid molecules which encode the MUC1 truncated receptor isoform peptides listed as SEQ ID NOS: 5, 6, 7, 8, and 9 in Table 1, or functional variants or fragments thereof, including, for example, the nucleotide sequences: SEQ ID NOS: 21, 22, 23, 24, and 25respectively, and

(b) nucleic acid molecules which hybridize under highly stringent conditions to the nucleic acid molecules of (a),

(c) deletions, additions and substitutions of the nucleic acid molecules of (a) or (b),

(d) nucleic acid molecules that differ from the nucleic acid molecules of (a), (b) or (c) in codon sequence due to the degeneracy of the genetic code, and

(e) complements of (a), (b), (c), or (d).

Certain isolated nucleic acids of the invention are nucleic acid molecules which encode a truncated isoform of the MUC1 receptor, or a functional fragment or variant thereof, or a functional equivalent thereof (e.g., a nucleic acid sequence encoding the same protein as encoded by one of the nucleic acid sequences, e.g. SEQ ID NO:21, listed in Table 2, provided that the functional fragment or equivalent encodes a protein which exhibits the functional activity of a truncated isoform of the MUC1 receptor encoded by such a listed sequence. As used herein, the functional activity of the truncated isoforms of the MUC1 receptor refers to the ability of the truncated isoforms of the MUC1 receptor peptide sequence to specifically interact with ligands for MGFR and to modulate cell growth or cell proliferation in response to such interaction. In certain embodiments, the isolated nucleic acid molecule is SEQ ID NO:21.

The invention provides nucleic acids and nucleic acid analog molecules which hybridize under high stringency conditions to a nucleic acid or nucleic acid analog molecule consisting of the nucleotide sequences set forth in SEQ ID NOS: 21-25. Such nucleic acid may be DNA, RNA, composed of mixed deoxyribonucleotides and ribonucleotides, or may also incorporate synthetic non-natural nucleotides. Various methods for determining the expression of a nucleic acid and/or a polypeptide in normal and tumor cells are known to those of skill in the art

The term “highly stringent conditions” or “high stringency conditions” as used herein refers to parameters with which those skilled in the art are familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH 7), 0.5% SDS, 2 mM EDTA). SSC is 0.15M sodium chloride/0.15M sodium citrate, pH 7; SDS is sodium dodecyl sulphate; and EDTA is ethylenediaminetetracetic acid. After hybridization, the membrane upon which the DNA is transferred is washed at 2×SSC at room temperature and then at 0.1×SSC/0.1×SDS at temperatures up to 68° C.

In general, homologs and alleles of a specific SEQ ID NO enumerated herein (see Table 2) typically will share at least 40% nucleotide identity and/or at least 50% amino acid identity to such a nucleotide sequence or amino acid sequence, respectively, in some instances will share at least 50% nucleotide identity and/or at least 65% amino acid identity and in still other instances will share at least 60% nucleotide identity and/or at least 75% amino acid identity. Preferred homologs and alleles share nucleotide and amino acid identities with SEQ ID NO:21 and SEQ ID NO:5, respectively; or SEQ ID NO:22 and SEQ ID NO:6, respectively; or SEQ ID NO:23 and SEQ ID NO:7, respectively; or SEQ ID NO:24 and SEQ ID NO:8, respectively; or SEQ ID NO:25 and SEQ ID NO:9, respectively; and encode polypeptides of greater than 80%, more preferably greater than 90%, still more preferably greater than 95% and most preferably greater than 99% identity. The percent identity can be calculated using various, publicly available software tools developed by NCBI (Bethesda, Md.) that can be obtained through the internet (ftp:/ncbi.nlm.nih.gov/pub/). Exemplary tools include the BLAST system available at http://www.ncbi.nlm.nih.gov, which uses algorithms developed by Altschul et al. (Nucleic Acids Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

The invention also provides isolated unique fragments of SEQ ID NOS: 21-25 and/or complements of SEQ ID NOS: 21-25. A unique fragment is one that is a ‘signature’ for the larger nucleic acid. It, for example, is long enough to assure that its precise sequence is not found in molecules outside of the inventive nucleic acid molecules defined above. Those of ordinary skill in the art may apply no more than routine procedures to determine if a fragment is unique within the human or mouse genome.

As will be recognized by those skilled in the art, the size of the above-mentioned unique fragment will depend upon its conservancy in the genetic code. Thus, some regions of SEQ ID NOS: 21-25 and their complements will require longer segments to be unique while others will require only short segments, typically between 12 and 32 nucleotides or more in length (e.g. 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115 or more), up to the entire length of the disclosed sequence. Many segments of the polynucleotide coding region or complements thereof that are 18 or more nucleotides in length will be unique. Those skilled in the art are well versed in methods for selecting such sequences, typically on the basis of the ability of the unique fragment to selectively distinguish the sequence of interest from other, unrelated nucleic acid molecules. A comparison of the sequence of the fragment to those on known data bases typically is all that is necessary, although in vitro confirmatory hybridization and sequencing analysis may be performed.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An “expression vector” is one into which a desired DNA sequence may be inserted by restriction and ligation such that it is operably joined to regulatory sequences and may be expressed as an RNA transcript. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays known in the art (e.g., β-galactosidase or alkaline phosphatase), and genes that visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. The precise nature of the regulatory sequences needed for gene expression may vary between species or cell types and are well-known to those of skill in the art.

Gene Therapy

In a specific embodiment, nucleic acids comprising sequences encoding MUC1, a fragment of MUC1 that is displayed on the cell surface, or the MGFR portion of MUC1 polypeptide are administered to treat, inhibit or prevent a disease or disorder in which immature cell therapy will benefit the patient, by way of gene therapy. Gene therapy refers to therapy performed by the administration to a subject of an expressed or expressible nucleic acid. In this embodiment of the invention, the nucleic acids produce their encoded protein that mediates a therapeutic effect by stimulating the proliferation of immature cells expressing MUC1.

Further, in an alternative embodiment, gene G-CSF receptor may be co-expressed for therapeutic purposes to stimulate proliferation of immature cells.

Any of the methods for gene therapy available in the art can be used according to the present invention. Exemplary methods are described below.

For general reviews of the methods of gene therapy, see Goldspiel et al., Clinical

Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, nucleic acid sequences may encode a MUC1, a fragment of MUC1 that is displayed on the cell surface, or the MGFR portion of MUC1 polypeptide, in which the nucleic acid sequences are part of expression vectors that express the polypeptides in a suitable host. In particular, such nucleic acid sequences have promoters operably linked to the polypeptide coding region, said promoter being inducible or constitutive, and, optionally, tissue-specific. In another particular embodiment, nucleic acid molecules are used in which the polypeptide coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors, or by direct injection of naked DNA, or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors) and so on. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor. Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding the polypeptide are used. The nucleic acid sequences encoding the polypeptide to be used in gene therapy are cloned into one or more vectors, which facilitates delivery of the gene into a patient. Retroviral vectors, adenoviral vectors and adeno-associated viruses are examples of viral vectors that may be used. Retroviral vectors contain the components necessary for the correct packaging of the viral genome and integration into the host cell DNA.

Adenoviruses are especially attractive vehicles for delivering genes to respiratory epithelia because they naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. In addition, adeno-associated virus (AAV) has also been proposed for use in gene therapy.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion and so on. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and so on.

The cell used for gene therapy may be autologous or allogeneic. In a preferred embodiment, the cell used for gene therapy is autologous to the patient.

In an embodiment in which recombinant cells are used in gene therapy, nucleic acid sequences encoding the polypeptide are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

In some embodiments, a virus vector for delivering a nucleic acid molecule encoding a peptide sequence of the invention is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol. 26:1951-1959, 1996). In certain embodiments, the virus vector is an adenovirus.

Another virus, which can potentially be used for certain applications, is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion

Other viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors can have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratoy Manual, W.H. Freeman Co., New York (1990) and Murry, E. J. Ed. “Methods in Molecular Biology,” vol. 7, Humana Press, Inc., Cliffton, N.J. (1991)

Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like.

For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

In addition to delivery through the use of vectors, nucleic acids of the invention may be delivered to cells without vectors, e.g. as “naked” nucleic acid delivery using methods known to those of skill in the art.

Transgenic Animal

According to another aspect of the invention, a transgenic non-human animal comprising an expression vector of the invention is provided. As used herein, “transgenic non-human animals” includes non-human animals having one or more exogenous nucleic acid molecules incorporated in germ line cells and/or somatic cells. Thus the transgenic animals include animals having episomal or chromosomally incorporated expression vectors, etc. In general, such expression vectors can use a variety of promoters which confer the desired gene expression pattern (e.g., temporal or spatial). Conditional promoters also can be operably linked to nucleic acid molecules of the invention to increase or decrease expression of the encoded polypeptide molecule in a regulated or conditional manner. Trans-acting negative or positive regulators of polypeptide activity or expression also can be operably linked to a conditional promoter as described above.

Administration and Dosage

In one particular embodiment, agents of the invention, which include antibodies, antibody fragments, proteins, peptides, nucleic acids and nucleic acid analogs, including DNA, RNA, RNAi, siRNA, shRNA and the like, are delivered to the desired site using nanoparticles. In one embodiment, the nanoparticle carries an antibody for targeting the nanoparticle to the desired site and also carries or delivers a therapeutic agent. Nanoparticle delivery of articles of the present invention may be used in vitro or in vivo. The preferred vehicle for delivery is a gold nanoparticle, optionally derivatized with either a polymer coating, cyclodextran, or a SAM.

When used therapeutically, the agents of the invention are administered in therapeutically effective amounts. In general, a therapeutically effective amount means that amount necessary to delay the onset of, inhibit the progression of, or halt altogether the particular condition being treated. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount typically varies from 0.01 mg/kg to about 1000 mg/kg. It is expected that dose ranging from 1-500 mg/kg, and preferably doses ranging from 1-50 mg/kg will be suitable. In other embodiments, the agents will be administered in doses ranging from 1 μg/kg/day to 10 mg/kg/day, with even more preferred doses ranging from 1-200 μg/kg/day, 1-100 μg/kg/day, 1-50 μg/kg/day or from 1-25 μg/kg/day. In other embodiments, dosages may range from about 0.1 mg/kg to about 200 mg/kg, and most preferably from about 0.2 mg/kg to about 20 mg/kg. These dosages can be applied in one or more dose administrations daily, for one or more days.

The agent of the invention should be administered for a length of time sufficient to provide either or both therapeutic and prophylactic benefit to the subject. Generally, the agent is administered for at least one day. In some instances, the agent may be administered for the remainder of the subject's life. The rate at which the agent is administered may vary depending upon the needs of the subject and the mode of administration. For example, it may be necessary in some instances to administer higher and more frequent doses of the agent to a subject for example during or immediately following a event associated with tumor or cancer, provided still that such doses achieve the medically desirable result. On the other hand, it may be desirable to administer lower doses in order to maintain the medically desirable result once it is achieved. In still other embodiments, the same dose of agent may be administered throughout the treatment period which as described herein may extend throughout the lifetime of the subject. The frequency of administration may vary depending upon the characteristics of the subject. The agent may be administered daily, every 2 days, every 3 days, every 4 days, every 5 days, every week, every 10 days, every 2 weeks, every month, or more, or any time there between as if such time was explicitly recited herein.

In one embodiment, daily doses of active agents will be from about 0.01 milligrams/kg per day to 1000 milligrams/kg per day. It is expected that oral doses in the range of 50 to 500 milligrams/kg, in one or several administrations per day, will yield the desired results. Dosage may be adjusted appropriately to achieve desired levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of agents.

Preferably, such agents are used in a dose, formulation and administration schedule which favor the activity of the agent and do not impact significantly, if at all, on normal cellular functions.

In one embodiment, the degree of activity of the agent is at least 10%. In other embodiments, the degree of activity of the drug is as least 20%, at least 30%, at least 40%, at least 50%, at least 60%, at least 70%, at least 80%, at least 90%, or at least 95%.

When administered to subjects for therapeutic purposes, the formulations of the invention are applied in pharmaceutically acceptable amounts and in pharmaceutically acceptable compositions. Such a pharmaceutical composition may include the agents of the invention in combination with any standard physiologically and/or pharmaceutically acceptable carriers which are known in the art. The compositions should be sterile and contain a therapeutically effective amount of the agent in a unit of weight or volume suitable for administration to a patient. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V)

Suitable preservatives include benzalkonium chloride (0.003-0.03% W/V); chlorobutanol (0.3-0.9% W/V); parabens (0.01-0.25% W/V) and thimerosal (0.004-0.02% W/V)

A variety of administration routes are available. The particular mode selected will depend, of course, upon the particular combination of drugs selected, the severity of the disease condition being treated, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, other mucosal forms, direct injection, transdermal, sublingual or other routes. “Parenteral” routes include subcutaneous, intravenous, intramuscular, or infusion. Direct injection may be preferred for local delivery to the site of the cancer. Oral administration may be preferred for prophylactic treatment e.g., in a subject at risk of developing a cancer, because of the convenience to the patient as well as the dosing schedule.

Chemical/physical vectors may be used to deliver the agents of the invention to a target (e.g. cell) and facilitate uptake thereby. As used herein, a “chemical/physical vector” refers to a natural or synthetic molecule, other than those derived from bacteriological or viral sources, capable of delivering the agent of the invention to a target (e.g. cell).

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. Nucleic acid and nucleic acid analogs, RNA, DNA, and intact virions can be encapsulated within the aqueous interior and be delivered to cells in a biologically active form (Fraley, et al., Trends Biochem. Sci., v. 6, p. 77 (1981)). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular (e.g. tissue), such as (e.g. the vascular cell wall), by coupling the liposome to a specific ligand such as a monoclonal antibody, sugar, glycolipid, or protein.

Liposomes are commercially available from Gibco BRL, for example, as LIPOFECTIN™. and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, V. 3, p. 235-241 (1985).

In one particular embodiment, the preferred vehicle is a biocompatible micro particle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”, claiming priority to U.S. patent application Ser. No. 213,668, filed Mar. 15, 1994). PCT/US/0307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the agent of the invention is encapsulated or dispersed within the biocompatible, preferably biodegradable polymeric matrix disclosed in PCT/US/03307. The polymeric matrix preferably is in the form of a micro particle such as a micro sphere (wherein the agent is dispersed throughout a solid polymeric matrix) or a microcapsule (wherein the agent is stored in the core of a polymeric shell). Other forms of the polymeric matrix for containing the agents of the invention include films, coatings, gels, implants, and stents. The size and composition of the polymeric matrix device is selected to result in favorable release kinetics in the tissue into which the matrix device is implanted. The size of the polymeric matrix devise further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the devise is administered to a vascular surface. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers arc preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Thus, the invention provides a composition of the above-described agents for use as a medicament, methods for preparing the medicament and methods for the sustained release of the medicament in vivo.

The compositions may conveniently be presented in unit dosage form and may be prepared by any of the methods well known in the art of pharmacy. All methods include the step of bringing the therapeutic agents into association with a carrier which constitutes one or more accessory ingredients. In general, the compositions are prepared by uniformly and intimately bringing the therapeutic agent into association with a liquid carrier, a finely divided solid carrier, or both, and then, if necessary, shaping the product

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion

Other delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include polymer based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; liposomes; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation

Use of a long-term sustained release implant may be particularly suitable for treatment of established disease conditions as well as subjects at risk of developing the disease. “Long-term” release, as used herein, means that the implant is constructed and arranged to deliver therapeutic levels of the active ingredient for at least 7 days, and preferably 30-60 days. The implant may be positioned at a site of injury or the location in which tissue or cellular regeneration is desired. Long-term sustained release implants are well known to those of ordinary skill in the art and include some of the release systems described above

The therapeutic agent may be administered in alone or in combination with other agents including proteins, receptors, co-receptors and/or genetic material designed to introduce into, upregulate or down regulate these genes in the area or in the cells. If the therapeutic agent is administered in combination the other agents may be administered by the same method, e.g. intravenous, oral, etc. or may be administered separately by different modes, e.g. therapeutic agent administered orally, administered intravenously, etc. In one embodiment of the invention the therapeutic agent and other agents are co-administered intravenously. In another embodiment the therapeutic agent and other agents are administered separately

Other agents that can be co-administered with the compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Antibody Production

Antibodies that bind to the MGFR portion of the MUC1 receptor, referred to herein as anti-PSMGFR are described in detail in PCT Application No. PCT/US2004/027954 (WO 2005/019269), in particular in Example 8 of the PCT Application. Antibody production is also described in PCT Application No. PCT/US2005/032821, in particular in Example 2 of the PCT Application. Inventive antibodies were raised against the PSMGFR portion of the MUC1 receptor, in particular nat-PSMGFR or var-PSMGFR shown in Table 1 using standard methods of antibody production. Rabbit polyclonal antibodies were produced and purified by column chromatography in which the immunizing peptide was attached to the chromatography column beads. The antibodies, anti-nat-PSMGFR and anti-var-PSMGFR, were shown to specifically and sensitively bind to the MGFR portion of the MUC1 receptor.

### Example 2

### Preparation of Tissue Specimens

Tissue specimens pictured in FIGS. 7-14 were prepared using methods previously described in PCT Application No. PCT/US2005/032821, in particular in Example 3 of the PCT Application. Formalin fixed, paraffin embedded tissue specimens were tested for reactivity to two antibodies that recognize different epitopes on the MUC1 receptor: 1) a rabbit polyclonal antibody, anti-PSMGFR, that binds to the PSMGFR portion of the MUC1 receptor that remains attached to the cell surface after receptor shedding; and 2) a commercially available mouse monoclonal, VU4H5 (Santa Cruz, Calif.) that binds to a sequence in the tandem repeat section of the receptor. One section from each block was stained with hemotoxin and eosin (H&E) to aid in assessing tumor grade.

### Example 3

### Induced Proliferation of Muc1-Presenting Cells

Methods used in FIGS. 2-4 are described in detail in PCT Application No. PCT/US2004/027954 (WO 2005/019269), in particular in Example 1 of the PCT Application. MUC1-positive cells were exposed to an inventive bivalent antibody grown against the MGFR region of the MUC1 receptor. Normalized cell growth was plotted as a function of antibody concentration. Bivalent antibodies were raised against either var-PSMGFR or nat-PSMGFR sequences shown in Table 1 (i.e., a single antibody having the ability to bind simultaneously to two MGFRs was produced). MUC1-positive breast tumor cells (T47Ds and 1504s), and a nat-PSMGFR transfected MUC1-negative cell line HEK293 were exposed to the antibody, and cell proliferation was studied as a function of concentration of the antibody. A growth/response curve typical of a growth factor/receptor—antibody response was observed. Specifically, at a concentration low enough that only a small portion of the cells were exposed to the antibody, cell proliferation was low. At a concentration of antibody high enough that one antibody could bind adjacent MGFRs, cell proliferation was maximized. At a high excess of antibody, each antibody bound only a single MGFR, rather than dimerizing adjacent MGFRs, and proliferation was reduced.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

TABLE 1

Peptide sequences (listed from N-terminus to
C-terminus):

Full-length MUC1 Receptor (Mucin 1 precursor,
Genbank Accession number: P15941)
MTPGTQSPFF LLLLLTVLTV VTGSGHASST PGGEKETSAT
QRSSVPSSTE KNAVSMTSSV LSSHSPGSGS STTQGQDVTL
APATEPASGS AATWGQDVTS VPVTRPALGS TTPPAHDVTS
APDNKPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDNRPALGS TAPPVHNVTS
ASGSASGSAS TLVHNGTSAR ATTTPASKST PFSIPSHHSD
TPTTLASHST KTDASSTHHS SVPPLTSSNH STSPQLSTGV
SFFFLSFHIS NLQFNSSLED PSTDYYQELQ RDISEMFLQI
YKQGGFLGLS NIKFRPGSVV VQLTLAFREG TINVHDVETQ
FNQYKTEAAS RYNLTISDVS VSDVPFPFSA QSGAGVPGWG
IALLVLVCVL VALAIVYLIA LAVCQCRRKN YGQLDIFPAR
DTYHPMSEYP TYHTHGRYVP PSSTDRSPYE KVSAGNGGSS
LSYTNPAVAA ASANL
(SEQ ID NO:1)
N-terminal MUC-1 signaling sequence for directing
MUC1 receptor and truncated isoforms to cell
membrane surface. Up to 3 amino acid residues may
be absent at C-terminal end as indicated by
variants in SEQ ID NOS:2, 3 and 4.
MTPGTQSPFFLLLLLTVLT
(SEQ ID NO:2).
MTPGTQSPFFLLLLLTVLT VVTA
(SEQ ID NO:3)
MTPGTQSPFFLLLLLTVLT VVTG
(SEQ ID NO:4)
A truncated MUC1 receptor isoform having
nat-PSMGFR at its N-terminus and including the
transmembrane and cytoplasmic sequences of a full-
length MUC1 receptor (“nat-PSMGFRTC isoform”-An
example of “PSMGFRTC”-shown excluding optional
N-terminus signal sequence, which may be cleaved
after translation and prior to expression of the
receptor on the cell surface):
G TINVHDVETQ FNQYKTEAAS RYNLTISDVS VSDVPFPFSA
QSGAGVPGWG IALLVLVCVL VALAIVYLIA LAVCQCRRKN
YGQLDIFPAR DTYHPMSEYP TYHTHGRYVP PSSTDRSPYE
KVSAGNGGSS LSYTNPAVAA ASANL
(SEQ ID NO:5)
A truncated MUC1 receptor isoform having
nat-PSMGFR and PSIBR at its N-terminus and
including the transmembrane and cytoplasmic
sequences of a full-length MUC1 receptor (“CM
isoform” shown excluding optional N-terminus
signal sequence, which may be cleaved after
translation and prior to expression of the
receptor on the cell surface):
GFLGLS NIKFRPGSVV VQLTLAFREG TINVHDVETQ FNQYKTEAAS
RYNLTISDVS VSDVPFPFSA QSGAGVPGWG IALLVLVCVL
VALAIVYLIA LAVCQCRRKN YGQLDIFPAR DTYHPMSEYP
TYHTHGRYVP PSSTDRSPYE KVSAGNGGSS LSYTNPAVAA
ASANL
(SEQ ID NO:6)
A truncated MUC1 receptor isoform having
nat-PSMGFR + PSIBR + Unique Region at its N-
terminus and including the transmembrane and
cytoplasmic sequences of a full-length MUC1
receptor (“UR isoform” shown excluding optional
N-terminus signal sequences):
ATTTPASKST PFSIPSHHSD TPTTLASHST KTDASSTHHS
TVPPLTSSNH STSPQLSTGV SFFFLSFHIS NLQFNSSLED
PSTDYYQELQ RDISEMFLQI YKQGGFLGLS NIKFRPGSVV
VQLTLAFREG TINVHDVETQ FNQYKTEAAS RYNLTISDVS
VSDVPFPFSA QSGAGVPGWG IALLVLVCVL VALAIVYLIA
LAVCQCRRKN YGQLDIFPAR DTYHPMSEYP TYHTHGRYVP
PSSTDRSPYE KVSAGNGGSS LSYTNPAVAA ASANL
(SEQ ID NO:7)
A truncated MUC1 receptor isoform including the
transmembrane and cytoplasmic sequences of a full-
length MUC1 receptor (“Y isoform” shown excluding
optional N-terminus signal sequence, which may be
cleaved after translation and prior to expression
of the receptor on the cell surface):
GSGHASSTPG GEKETSATQR SSVPSSTEKN AFNSSLEDPS
TDYYQELQRD ISEMFLQIYK QGGFLGLSNI KFRPGSVVVQ
LTLAFREGTI NVHDMETQFN QYKTEAASRY NLTISDVSVS
DVPFPFSAQS GAGVPGWGIA LLVLVCVLVA LAIVYLIALA
VCQCRRKNYG QLDIFPARDT YHPMSEYPTY HTHGRYVPPS
STDRSPYEKV SAGNGGSSLS YTNPAVAATS ANL
(SEQ ID NO:8)
A truncated MUC1 receptor isoform having
nat-PSMGFR + PSIBR + Unique Region + Repeats at
its N-terminus and including the transmembrane and
cytoplasmic sequences of a full-length MUC1
receptor (“Rep isoform” shown excluding optional
N-terminus signal sequence, which may be cleaved
after translation and prior to expression of the
receptor on the cell surface):
LDPRVRTSAP DTRPAPGSTA PQAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DTRPAPGSTA
PPAHGVTSAP DTRPAPGSTA PPAHGVTSAP DNRPALGSTA
PPVHNVTSAS GSASGSASTL VHNGTSARAT TTPASKSTPF
SIPSHHSDTP TTLASHSTKT DASSTHHSSV PPLTSSNHST
SPQLSTGVSF FFLSFHISNL QFNSSLEDPS TDYYQELQRD
ISEMFLQIYK QGGFLGLSNI KFRPGSVVVQ LTLAFREGTI
NVHDVETQFN QYKTEAASRY NLTISDVSVS DVPFPFSAQS
GAGVPGWGIA LLVLVCVLVA LAIVYLIALA VCQCRRKNYG
QLDIFPARDT YHPMSEYPTY HTHGRYVPPS STDRSPYEKV
SAGNGGSSLS YTNPAVAAAS ANL
(SEQ ID NO:9)
Native Primary Sequence of the MUC1 Growth Factor
Receptor (nat-PSMGFR-an example of “PSMGFR”):
GTINVHDVETQFNQYKTEAASRYNLTISDVSVSDVPFPFSAQSGA
(SEQ ID NO:10)
Native Primary Sequence of the MUC1 Growth Factor
Receptor (nat-PSMGFR-An example of “PSMGFR”),
having a single amino acid deletion at the
N-terminus of SEQ ID NO:10):
TINVHDVETQFNQYKTEAASRYNLTISDVSVSDVPFPFSAQSGA
(SEQ ID NO:11)
“SPY” functional variant of the native Primary
Sequence of the MUC1 Growth Factor Receptor having
enhanced stability (var-PSMGFR-An example of
“PSMGFR”):
GTINVHDVETQFNQYKTEAASPYNLTISDVSVSDVPFPFSAQSGA
(SEQ ID NO:12)
“SPY” functional variant of the native Primary
Sequence of the MUC1 Growth Factor Receptor having
enhanced stability (var-PSMGFR-An example of
“PSMGFR”), having a single amino acid deletion
at the C-terminus of SEQ ID NO:12):
TINVHDVETQFNQYKTEAASPYNLTISDVSVSDVPFPFSAQSGA
(SEQ ID NO:13)
Truncated PSMGFR receptor (TR) (having “SPY”
sequence of var-PSMGFR):
GTINVHDVETQFNQYKTEAASPYNLTISDVSVS
(SEQ ID NO:14)
Extended Sequence of MUC1 Growth Factor Receptor
(ESMGFR) (having ”SPY“ sequence of var-PSMGFR):
VQLTLAFREGTINVHDVETQFNQYKTEAA“SPY” NLTISDVSVSDVPFPF
(SEQ ID NO:15)
Tumor-Specific Extended Sequence of MUC1 Growth
Factor Receptor (TSESMGFR) (having “SPY” sequence
of var-PSMGFR):
SVVVQLTLAGREGTINVHDVETQFNQYKTEAASPYNLTISDVSVSDVPFP
FSAQSGA
(SEQ ID NO:16)
Primary Sequence of the Interchain Binding Region)
(PSIBR):
GFLGLSNIKFRPGSVVVQLTLAFRE
(SEQ ID NO:17)
Truncated Interchain Binding Region) (TPSIBR):
SVVVQLTLAFREG
(SEQ ID NO:18)
Repeat Motif 2 (RM2):
PDTRPAPGSTAPPAHGVTSAPDTRPAPGSTAPPAHGVTSA/
(SEQ IDNO:19)

TABLE 2
Nucleic acid sequences encoding for truncated
isoforms of MUC1 receptor (listed from 5′-
terminus to 3′-terminus):

An example of a nucleic acid molecule encoding the
full-length MUC1 receptor of SEQ ID NO:1:

(SEQ ID NO:20)

acaggttctggtcatgcaagctctaccccaggtggagaaaaggagacttc
ggctacccagagaagttcagtgcccagctctactgagaagaatgctgtga
gtatgaccagcagcgtactctccagccacagccccggttcaggctcctcc
accactcagggacaggatgtcactctggccccggccacggaaccagcttc
aggttcagctgccacctggggacaggatgtcacctcggtcccagtcacca
ggccagccctgggctccaccaccccgccagcccacgatgtcacctcagcc
ccggacaacaagccagccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccacggtgt
cacctcggccccggacaccaggccggccccgggctccaccgcccccccag
cccacggtgtcacctcggccccggacaccaggccggccccgggctccacc
gcccccccagcccacggtgtcacctcggccccggacaccaggccggcccc
gggctccaccgcccccccagcccacggtgtcacctcggccccggacacca
ggccggccccgggctccaccgcccccccagcccacggtgtcacctcggcc
ccggacaccaggccggccccgggctccaccgcccccccagcccatggtgt
cacctcggccccggacaacaggcccgccttgggctccaccgcccctccag
tccacaatgtcacctcggcctcaggctctgcatcaggctcagcttctact
ctggtgcacaacggcacctctgccagggctaccacaaccccagccagcaa
gagcactccattctcaattcccagccaccactctgatactcctaccaccc
ttgccagccatagcaccaagactgatgccagtagcactcaccatagctcg
gtacctcctctcacctcctccaatcacagcacttctccccagttgtctac
tggggtctctttctttttcctgtcttttcacatttcaaacctccagttta
attcctctctggaagatcccagcaccgactactaccaagagctgcagaga
gacatttctgaaatgtttttgcagatttataaacaagggggttttctggg
cctctccaatattaagttcaggccaggatctgtggtggtacaattgactc
tggccttccgagaaggtaccatcaatgtccacgacgtggagacacagttc
aatcagtataaaacggaagcagcctctcgatataacctgacgatctcaga
cgtcagcgtgagtgatgtgccatttccttctctgcccagtctggggctgg
ggtgccaggctggggcatcgcgctgctggtgctggtctgtgttctggttg
cgctggccattgtctatctcattgccttggctgtctgtcagtgccgccga
aagaactacgggcagctggacatctttccagcccgggatacctaccatcc
tatgagcgagtaccccacctaccacacccatgggcgctatgtgcccccta
gcagtaccgatcgtagcccctatgagaaggtttctgcaggtaacggtggc
agcagcctctcttacacaaacccagcagtggcagccgcttctgccaactt
gtagggcacgtcgccgctgagctgagtggccagccagtgccattccactc
cactcaggttcttcaggccagagcccctgcaccctgtttgggctggtgag
ctgggagttcaggtgggctgctcacagcctccttcagaggccccaccaat
ttctcggacacttctcagtgtgtggaagctcatgtgggcccctgaggctc
atgcctgggaagtgttgtgggggctcccaggaggactggcccagagagcc
ctgagatagcggggatcctgaactggactgaataaaacgtggtctcccac
tg
An example of a nucleic acid molecule encoding the
nat-PSMGFRTC of SEQ ID NO:5:

(SEQ ID NO:21)

Acgggcacggccggtaccatcaatgtccacgacgtggagacacagttcaa
tcagtataaaacggaagcagcctctcgatataacctgacgatctcagacg
tcagcgtgagtgatgtgccatttcctttctctgcccagtctggggctggg
gtgccaggctggggcatcgcgctgctggtgctggtctgtgttctggttgc
gctggccattgtctatctcattgccttggctgtctgtcagtgccgccgaa
agaactacgggcagctggacatctttccagcccgggatacctaccatcct
atgagcgagtaccccacctaccacacccatgggcgctatgtgccccctag
cagtaccgatcgtagcccctatgagaaggtttctgcaggtaacggtggca
gcagcctctcttacacaaacccagcagtggcagccgcttctgccaacttg
tagggcacgtcgccgctgagctgagtggccagccagtgccattccactcc
actcaggttcttcaggccagagcccctgcaccctgtttgggctggtgagc
tgggagttcaggtgggctgctcacagcctccttcagaggccccaccaatt
tctcggacacttctcagtgtgtggaagctcatgtgggcccctgaggctca
tgcctgggaagtgttgtgggggctcccaggaggactggcccagagagccc
tgagatagcggggatcctgaactggactgaataaaacgtggtctcccact
g
An example of a nucleic acid molecule encoding the
CM isoform of SEQ ID NO:6:

(SEQ ID NO:22)

Acggccggttttctgggcctctccaatattaagttcaggccaggatctgt
ggtggtacaattgactctggccttccgagaaggtaccatcaatgtccacg
acgtggagacacagttcaatcagtataaaacggaagcagcctctcgatat
aacctgacgatctcagacgtcagcgtgagtgatgtgccatttcctttctc
tgcccagtctggggctggggtgccaggctggggcatcgcgctgctggtgc
tggtctgtgttctggttgcgctggccattgtctatctcattgccttggct
gtctgtcagtgccgccgaaagaactacgggcagctggacatctttccagc
ccgggatacctaccatcctatgagcgagtaccccacctaccacacccatg
ggcgctatgtgccccctagcagtaccgatcgtagcccctatgagaaggtt
tctgcaggtaacggtggcagcagcctctcttacacaaacccagcagtggc
agccgcttctgccaacttgtagggcacgtcgccgctgagctgagtggcca
gccagtgccattccactccactcaggttcttcaggccagagcccctgcac
cctgtttgggctggtgagctgggagttcaggtgggctgctcacagcctcc
ttcagaggccccaccaatttctcggacacttctcagtgtgtggaagctca
tgtgggcccctgaggctcatgcctgggaagtgttgtgggggctcccagga
ggactggcccagagagccctgagatagcggggatcctgaactggactgaa
taaaacgtggtctcccactg
An example of a nucleic acid molecule encoding the
UR isoform of SEQ ID NO:7:

(SEQ ID NO:23)

Acggccgctaccacaaccccagccagcaagagcactccattctcaattcc
cagccaccactctgatactcctaccacccttgccagccatagcaccaaga
ctgatgccagtagcactcaccatagctcggtacctcctctcacctcctcc
aatcacagcacttctccccagttgtctactggggtctctttctttttcct
gtcttttcacatttcaaacctccagtttaattcctctctggaagatccca
gcaccgactactaccaagagctgcagagagacatttctgaaatgtttttg
cagatttataaacaagggggttttctgggcctctccaatattaagttcag
gccaggatctgtggtggtacaattgactctggccttccgagaaggtacca
tcaatgtccacgacgtggagacacagttcaatcagtataaaacggaagca
gcctctcgatataacctgacgatctcagacgtcagcgtgagtgatgtgcc
atttcctttctctgcccagtctggggctggggtgccaggctggggcatcg
cgctgctggtgctggtctgtgttctggttgcgctggccattgtctatctc
attgccttggctgtctgtcagtgccgccgaaagaactacgggcagctgga
catctttccagcccgggatacctaccatcctatgagcgagtaccccacct
accacacccatgggcgctatgtgccccctagcagtaccgatcgtagcccc
tatgagaaggtttctgcaggtaacggtggcagcagcctctcttacacaaa
cccagcagtggcagccgcttctgccaacttgtagggcacgtcgccgctga
gctgagtggccagccagtgccattccactccactcaggttcttcaggcca
gagcccctgcaccctgtttgggctggtgagctgggagttcaggtgggctg
ctcacagcctccttcagaggccccaccaatttctcggacacttctcagtg
tgtggaagctcatgtgggcccctgaggctcatgcctgggaagtgttgtgg
gggctcccaggaggactggcccagagagccctgagatagcggggatcctg
aactggactgaataaaacgtggtctcccactg
An example of a nucleic acid molecule encoding the
Y isoform of SEQ ID NO:8:

(SEQ ID NO:24)

Acaggttctggtcatgcaagctctaccccaggtggagaaaaggagacttc
ggctacccagagaagttcagtgcccagctctactgagaagaatgctttta
attcctctctggaagatcccagcaccgactactaccaagagctgcagaga
gacatttctgaaatgtttttgcagatttataaacaagggggttttctggg
cctctccaatattaagttcaggccaggatctgtggtggtacaattgactc
tggccttccgagaaggtaccatcaatgtccacgacgtggagacacagttc
aatcagtataaaacggaagcagcctctcgatataacctgacgatctcaga
cgtcagcgtgagtgatgtgccatttcctttctctgcccagtctggggctg
gggtgccaggctggggcatcgcgctgctggtgctggtctgtgttctggtt
gcgctggccattgtctatctcattgccttggctgtctgtcagtgccgccg
aaagaactacgggcagctggacatctttccagcccgggatacctaccatc
ctatgagcgagtaccccacctaccacacccatgggcgctatgtgccccct
agcagtaccgatcgtagcccctatgagaaggtttctgcaggtaatggtgg
cagcagcctctcttacacaaacccagcagtggcagccacttctgccaact
tgtaggggcacgtcgcc
An example of a nucleic acid molecule encoding the
Rep isoform of SEQ ID NO:9:

(SEQ ID NO:25)

ctcgacccacgcgtccgctcgacccacgcgtccgcacctcggccccggac
accaggccggccccgggctccaccgcccccccagcccacggtgtcacctc
ggccccggacaccaggccggccccgggctccaccgcccccccagcccacg
gtgtcacctcggccccggacaccaggccggccccgggctccaccgccccc
ccagcccacggtgtcacctcggccccggacaccaggccggccccgggctc
caccgcccccccagcccacggtgtcacctcggccccggacaccaggccgg
ccccgggctccaccgcccccccagcccacggtgtcacctcggccccggac
accaggccggccccgggctccaccgcccccccagcccacggtgtcacctc
ggccccggacaccaggccggccccgggctccaccgcccccccagcccacg
gtgtcacctcggccccggacaccaggccggccccgggctccaccgccccc
ccagcccacggtgtcacctcggccccggacaccaggccggccccgggctc
caccgcccccccagcccacggtgtcacctcggccccggacaccaggccgg
ccccgggctccaccgcccccccagcccacggtgtcacctcggccccggac
accaggccggccccgggctccaccgcccccccagcccacggtgtcacctc
ggccccggacaccaggccggccccgggctccaccgcccccccagcccacg
gtgtcacctcggccccggacaccaggccggccccgggctccaccgccccc
ccagcccacggtgtcacctcggccccggacaccaggccggccccgggctc
caccgcccccccagcccacggtgtcacctcggccccggacaccaggccgg
ccccgggctccaccgcccccccagcccacggtgtcacctcggccccggac
accaggccggccccgggctccaccgcccccccagcccacggtgtcacctc
ggccccggacaccaggccggccccgggctccaccgcccccccagcccacg
gtgtcacctcggccccggacaccaggccggccccgggctccaccgccccc
ccagcccacggtgtcacctcggccccggacaccaggccggccccgggctc
caccgcccccccagcccacggtgtcacctcggccccggacaccaggccgg
ccccgggctccaccgcccccccagcccatggtgtcacctcggccccggac
aacaggcccgccttgggctccaccgcccctccagtccacaatgtcacctc
ggcctcaggctctgcatcaggctcagcttctactctggtgcacaacggca
cctctgccagggctaccacaaccccagccagcaagagcactccattctca
attcccagccaccactctgatactcctaccacccttgccagccatagcac
caagactgatgccagtagcactcaccatagctcggtacctcctctcacct
cctccaatcacagcacttctccccagttgtctactggggtctctttcttt
ttcctgtcttttcacatttcaaacctccagtttaattcctctctggaaga
tcccagcaccgactactaccaagagctgcagagagacatttctgaaatgt
ttttgcagatttataaacaagggggttttctgggcctctccaatattaag
ttcaggccaggatctgtggtggtacaattgactctggccttccgagaagg
taccatcaatgtccacgacgtggagacacagttcaatcagtataaaacgg
aagcagcctctcgatataacctgacgatctcagacgtcagcgtgagtgat
gtgccatttcctttctctgcccagtctggggctggggtgccaggctgggg
catcgcgctgctggtgctggtctgtgttctggttgcgctggccattgtct
atctcattgccttggctgtctgtcagtgccgccgaaagaactacgggcag
ctggacatctttccagcccgggatacctaccatcctatgagcgagtaccc
cacctaccacacccatgggcgctatgtgccccctagcagtaccgatcgta
gcccctatgagaaggtttctgcaggtaacggtggcagcagcctctcttac
acaaacccagcagtggcagccgcttctgccaacttgtagggcacgtcgcc
gctgagctgagtggccagccagtgccattccactccactcaggttcttca
ggccagagcccctgcaccctgtttgggctggtgagctgggagttcaggtg
ggctgctcacagcctccttcagaggccccaccaatttctcggacacttct
cagtgtgtggaagctcatgtgggcccctgaggctcatgcctgggaagtgt
tgtgggggctcccaggaggactggcccagagagccctgagatagcgggga
tcctgaactggactgaataaaacgtggtctcccactg

TABLE 3

| Cell Type | Reference | Indication |
|---|---|---|
| Immature Erythrocytes | Regulated expression of | Treatment of Blood |
| | MUC1 epithelial antigen in | diseases, anemia |
| | erythropoiesis. Rughetti A, | |
| | Biffoni M, Pierelli L, | |
| | Rahimi H, Bonanno G, | |
| | Barachini S, Pellicciotta I, | |
| | Napoletano C, Pescarmona | |
| | E, Del Nero A, Pignoloni P, | |
| | Frati L and Nuti M. (2003) | |
| | Br. J. Haematol, 120(2): | |
| | 344-352 | |
| Dendritic Cells | Mucin-1 is expressed on | Treatment of Immune |
| | dendritic cells, both in vitro | diseases, especially |
| | and in vivo. Cloosen S, | immune-deficiency diseases |
| | Thio M, Vanclee A, van | |
| | Leeuwen E B, Senden- | |
| | Gijsbers B L, Oving E B, | |
| | Germeraad W T, Bos G M. | |
| | (2004) Int. Immunol. 11, | |
| | 1561-71 | |
| Epithelial Progenitor Cells | Epithelial progenitors in the | Tissue regeneration |
| | normal human mammary | Tissue augmentation |
| | gland. Stingl J, Raouf A, | |
| | Emerman J T, Eaves C J. J | |
| | Mammary Gland Biol | |
| | Neoplasia. (2005) | |
| | Jan; 10(1): 49-59. | |
| Monoblasts and Monocytes | Epithelial membrane | Treatment of patients |
| | antigen (EMA) or MUC1 | following chemotherapy |
| | expression in monocytes | and/or radiation therapy |
| | and monoblasts. Leong C F, | Other conditions in which it |
| | Raudhawati O, Cheong S K, | is desirable to augment |
| | Sivagengi K and Noor | macrophage lineage |
| | Hamdiah H. 2003 | |
| | Pathology, 35, 422-427 | |
| Endometrial Cells | Human endometrial mucin | For the treatment of |
| | MUC1 is up-regulated by | endometriosis, and other |
| | progesterone and down- | fertility related conditions |
| | regulated in vitro by the | |
| | human blastocyst. | |
| | Meseguer M, Aplin J D, | |
| | Caballero-Campo P, | |
| | O'Connor J E, Martin J C, | |
| | Remohi J, Pellicer A, | |
| | Simon C. (2001) Biol. | |
| | Reprod. 64(2) 590-601 | |
| Pneumocyte | MUC1 is a novel marker for | For the treatment of |
| | the type II pneumocyte | respiratory diseases |
| | lineage during lung | |
| | carcinogenesis. J A Jarrard, | |
| | R I Linnoila, H Lee, S M | |
| | Steinberg, H Witschi and E | |
| | Szabo. (1998) Cancer | |
| | Research, 58, (23) 5582- | |
| | 5589 | |
| Neutrophils and Precursors | G-CSF induces elevation of | For the treatment of blood |
| | circulating CA 15-3 in | diseases, and Neutropenia |
| | breast carcinoma patients | For the treatment of patients |
| | treated in an adjuvant | receiving ablative radiation, |
| | setting. Briasoulis E, | to replace bone marrow |
| | Andreopolou E, Tolis C F, | transplantation |
| | Bairaktari E, Katsaraki A, | |
| | Dimopoulos M A, | |
| | Fountzilas G, Seferiadis C | |
| | ans Pavlidis N. (2001) | |
| | Cancer, 91, 909-917 | |
| Mast progenitor Cells | Applicant | For the treatment of |
| | | immuno-compromised |
| | | patients |

1. A method of isolating or selecting stem cells from a mixed population containing stem cells, comprising contacting the population of cells with a ligand specific for a truncated MUC1 receptor, wherein the presence of the truncated MUC1 receptor on the cells indicates that they are stem cells.

2. The method according to claim 1, wherein the truncated MUC1 receptor region is MGFR.

3. The method according to claim 2, wherein MGFR is a region consisting essentially of an amino acid sequence of PSMGFR.

4. The method according to claim 1, wherein the stem cell is pluripotent or embryonic.

5. The method according to claim 1, wherein the stem cell is an adult stem cell.

6. The method according to claim 5, wherein the stem cell is a hematopoietic or stromal cell.

7. The method according to claim 6, wherein the stem cells can differentiate into erythrocytes or neutrophils.

8. The method according to claim 1, further comprising contacting the population of cells with known stem cell markers.

9. The method according to claim 8, wherein the known stem cell markers are specific to differentiation lineages.

10. A method of isolating or selecting progenitor cells from a mixed population containing the progenitor cells, comprising contacting the population of cells with a ligand specific for a truncated MUC1 receptor, wherein the presence of the truncated MUC1 receptor on the cells indicates that they can be multiplied or can undergo another step of differentiation.

11. The method according to claim 1, wherein the truncated MUC1 receptor region is MGFR, which consists of at least 15 contiguous amino acids within the PSMGFR region.

12. The method according to claim 1, wherein the progenitor cell is a precursor to “blood cells”.

13. The method according to claim 12, wherein the progenitor cells differentiate into erythrocytes, neutrophils, mast cells, monocytes, NK cells, T-cells, or B-cells.

14. The method according to claim 1, further comprising contacting the population of cells with known progenitor cell markers.

15. The method according to claim 14, wherein the known stem cell markers are specific to differentiation lineages.

16. A method of amplifying a population of stem cells or progenitor cells comprising contacting the cells with a bi-valent ligand that binds to truncated MUC1 receptor.

17. A method of inducing tumor cell death comprising contacting tumor cells with a mono-valent ligand specific for a truncated MUC1 receptor.

18. The method according to claim 17, wherein the truncated MUC1 receptor is MGFR, which consists of at least 15 contiguous amino acids within the PSMGFR region.

19. The method according to claim 18, wherein the ligand is a monovalent or bi-specific antibody.

20. A method for inducing tumor cell death, wherein a non-dimer form of a protein belonging to NM23 family of proteins contacts truncated MUC1 receptor in a tumor cell.

21. The method according to claim 20, wherein the truncated receptor is MGFR, which consists of at least 15 contiguous amino acids within the PSMGFR region.

22. The method according to claim 20, wherein the protein is NM23.

23. The method according to claim 20, comprising contacting the cell with an NM23 mutant that results in multimerization of NM23, but not dimerization.

24. The method for inducing tumor cell death according to claim 22, wherein the NM23 is H1 isoform.

25. A method of expanding a population of stem cells or progenitor cells, comprising contacting the cells with a dimer of a family of proteins belonging to NM23 family.

26. The method according to claim 25, wherein the stem cells are hematopoietic stem cells.

27. The method according to claim 26, wherein the protein is mutant NM23, which prefers dimer formation to higher order multimers.

28. The method according to claim 27, wherein the mutant NM23 is S120G (Kim et al., Biochem. Biophys Res. Comm. 2003, 307; 281-289.)

29. The method according to claim 25, wherein the truncated MUC1 receptor form is MGFR, which consists of at least 15 contiguous amino acids within the PSMGFR region.

30. The method according to claim 25, wherein the NM23 is H1 isoform.

31. A method of treating symptoms of cancer in a subject comprising administering a gene encoding NM23 in the subject suffering from cancer.

32. A mutant NM23 protein that prefers hexamer formation.

33. A method for detecting a subject who has susceptibility to developing cancer, comprising screening for NM23 mutation in the subject, wherein the presence of a mutation that forms NM23 dimers, indicates that the subject is susceptible to developing cancer.

34. The method according to claim 33, wherein the screening is carried out on blood or other bodily fluid.

35. A method for detecting a subject who has susceptibility to developing cancer, comprising screening for the presence of MMP14 or ADAM17 in the subject, wherein its presence indicates that the subject is susceptible to developing cancer.

36. The method according to claim 10, wherein the progenitor cells are pluripotent hematopoietic stem cell, common lymphoid progenitor, common myeloid progenitor, granulocyte/macrophage progenitor, mast cell progenitor, or erythrocyte/platelet progenitor.

37. A method of modulating growth of stem cells by manipulating MUC1 protein by contacting cells with an agent.

38. The method according to claim 37, wherein the growth of stem cells is modulated by contacting cells with an agent that binds to the MGFR portion of the MUC1 protein.

39. The method according to claim 38, wherein growth is stimulated by contacting cells with agents that dimerize the MGFR portion of MUC1.

40. The method according to claim 39, wherein the agent is a bivalent antibody or NM23.

41. The method according to claim 37, wherein the stem cells are embryonic stem cells.

42. The method according to claim 37, wherein the growth of stem cells is inhibited by contacting cells with an agent that binds to the MGFR portion of MUC1 and blocks its dimerization.

43. The method according to claim 42, wherein the stem cells are cancer stem cells.

44. The method according to claim 42, wherein the agent is a monovalent antibody that binds to the MGFR portion of MUC1.

45. The method according to claim 44, wherein the antibody binds to the PSMGFR or PFMGFR-nat peptide.

46. The method according to claim 37, wherein said modulation is differentiation, which is induced by contacting the cells with a agent that inhibits MUC1 cleavage; suppress expression of MMP-14 or TACE; suppress expression of NM23; or suppress expression of bFGF.

47. The method according to claim 37, wherein the agent is delivered by a nanoparticle.

48. The method according to claim 37, wherein the agent is co-immobilized on a nanoparticle that also presents an agent that binds to MUC1.

49. The method according to claim 48, wherein the nanoparticle is gold.

50. The method according to claim 49, wherein the nanoparticle is coated with self assembled monolayer (SAM).

51. A method of identifying and selecting stem cells by contacting a population of cells with an antibody that binds to MUC1.

52. The method according to claim 51, wherein the antibody binds to MGFR portion of MUC1.

53. The method according to claim 52, wherein a level of binding of a first antibody that binds to the MGFR portion of MUC1 is compared to a level of binding of a second antibody that binds to the full-length MUC1 protein is used to identify and select stem cells, wherein undifferentiated stem cells are identified and selected by isolating those cells that have high levels of MGFR and low levels of full-length MUC1.

54. The method according to claim 51, wherein the stem cells are totipotent cells, pluripotent cells or cancer stem cells.

55. A kit comprising a reagent to stimulate stem cell growth comprising: an antibody that binds to MGFR portion of MUC1; and/or NM23; and/or
RNAi that inhibits agents that suppress the expression of MMP-14 or TACE.

56. The kit according to claim 55, wherein reagent is present in or on a nanoparticle.

57. A kit comprising a reagent for initiating differentiation of a cell, comprising: an antibody that binds to a surface marker of differentiation including SSEA4, Tra 1-81, or Tra 1-60; and/or RNAi that suppresses expression of MMP-14 and/or TACE.

58. The kit according to claim 55, wherein reagent is present in or on a nanoparticle.


SEQUENCE LISTING (25 sequences)

SEQ ID NO: 1 - protein, 1255 aa, Artificial Sequence
  Full length MUC1 Receptor
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 2 - protein, 19 aa, Artificial Sequence
  N-terminal MUC-1
MTPGTQSPFFLLLLLTVLT

SEQ ID NO: 3 - protein, 23 aa, Artificial Sequence
  N-terminal MUC-1
MTPGTQSPFFLLLLLTVLTVVTA

SEQ ID NO: 4 - protein, 23 aa, Artificial Sequence
  N-terminal MUC-1
MTPGTQSPFFLLLLLTVLTVVTG

SEQ ID NO: 5 - protein, 146 aa, Artificial Sequence
  Fragment of MUC1 Receptor
GTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 6 - protein, 171 aa, Artificial Sequence
  Fragment of MUC-1 Receptor
GFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 7 - protein, 275 aa, Artificial Sequence
  Fragment of MUC-1 Receptor
ATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 8 - protein, 233 aa, Artificial Sequence
  Fragment of MUC1 Receptor
GSGHASSTPGGEKETSATQRSSVPSSTEKNAFNSSLEDPSTDYYQELQRDISEMFLQIYKQGGFLGLSNIKFRPGSVVVQLTLAFREGTINVHDMETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 9 - protein, 863 aa, Artificial Sequence
  Fragment of MUC1 Receptor
LDPRVRTSAPDTRPAPGSTAPQAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 10 - protein, 45 aa, Artificial Sequence
  Fragment of MUC1 Growth Factor Receptor
GTINVHDVETQFNQYKTEAASRYNLTISDVSVSDVPFPFSAQSGA

SEQ ID NO: 11 - protein, 44 aa, Artificial Sequence
  Fragment of MUC1 Growth Factor Receptor
TINVHDVETQFNQYKTEAASRYNLTISDVSVSDVPFPFSAQSGA

SEQ ID NO: 12 - protein, 45 aa, Artificial Sequence
  Variant of Fragment of MUC1 Growth Factor Receptor
GTINVHDVETQFNQYKTEAASPYNLTISDVSVSDVPFPFSAQSGA

SEQ ID NO: 13 - protein, 44 aa, Artificial Sequence
  Variant of Fragment of MUC1 Growth Factor Receptor
TINVHDVETQFNQYKTEAASPYNLTISDVSVSDVPFPFSAQSGA

SEQ ID NO: 14 - protein, 33 aa, Artificial Sequence
  Variant of Fragment of MUC1 Growth Factor Receptor
GTINVHDVETQFNQYKTEAASPYNLTISDVSVS

SEQ ID NO: 15 - protein, 48 aa, Artificial Sequence
  Variant of Fragment of MUC1 Receptor
VQLTLAFREGTINVHDVETQFNQYKTEAASPYNLTISDVSVSDVPFPF

SEQ ID NO: 16 - protein, 57 aa, Artificial Sequence
  Variant of Fragment of MUC1 Receptor
SVVVQLTLAFREGTINVHDVETQFNQYKTEAASPYNLTISDVSVSDVPFPFSAQSGA

SEQ ID NO: 17 - protein, 25 aa, Artificial Sequence
  Interchain Binding Region of MUC1 Receptor
GFLGLSNIKFRPGSVVVQLTLAFRE

SEQ ID NO: 18 - protein, 13 aa, Artificial Sequence
  Truncated Interchain Binding Region of MUC1 Receptor
SVVVQLTLAFREG

SEQ ID NO: 19 - protein, 40 aa, Artificial Sequence
  Repeat Motif 2 of MUC1 Receptor
PDTRPAPGSTAPPAHGVTSAPDTRPAPGSTAPPAHGVTSA

SEQ ID NO: 20 - DNA, 4003 bp, Homo sapiens
acaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 21 - DNA, 751 bp, Homo sapiens
acgggcacggccggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 22 - DNA, 820 bp, Homo sapiens
acggccggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 23 - DNA, 1132 bp, Homo sapiens
acggccgctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 24 - DNA, 717 bp, Homo sapiens
acaggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcc

SEQ ID NO: 25 - DNA, 2487 bp, Homo sapiens
ctcgacccacgcgtccgctcgacccacgcgtccgcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg